SEQUENCE LISTING (105 sequences)

SEQ ID NO: 1 - protein, 120 aa, Artificial sequence
  Synthetic H7 heavy chain
QVQLQESGGGVVQPGRSLRLSCAASRFTFSSYAMHWVRQAPGKGLEWVAVISYDGSNKYYADSVKGRFTISRDNSKNTLYLQMNSLRAEDTAVYYCARDLSGYGDYPDYWGQGTLVTVSS

SEQ ID NO: 2 - protein, 108 aa, Artificial sequence
  Synthetic H7 light chain
SELTQDPAVSVALGQTVRITCQGDSLRSYYASWYQQKPGQAPVLVMYGRNERPSGVPDRFSGSKSGTSASLAISGLQPEDEANYYCAGWDDSLTGPVFGGGTKLTVLG

SEQ ID NO: 3 - protein, 15 aa, Artificial sequence
  Synthetic Linker scFv format
GGGGSGGGGSGGGGS

SEQ ID NO: 4 - protein, 243 aa, Artificial sequence
  Synthetic H7
QVQLQESGGGVVQPGRSLRLSCAASRFTFSSYAMHWVRQAPGKGLEWVAVISYDGSNKYYADSVKGRFTISRDNSKNTLYLQMNSLRAEDTAVYYCARDLSGYGDYPDYWGQGTLVTVSSGGGGSGGGGSGGGGSSELTQDPAVSVALGQTVRITCQGDSLRSYYASWYQQKPGQAPVLVMYGRNERPSGVPDRFSGSKSGTSASLAISGLQPEDEANYYCAGWDDSLTGPVFGGGTKLTVLG

SEQ ID NO: 5 - protein, 120 aa, Artificial sequence
  Synthetic heavy chain F12
QVQLQESGGGLVQPGGSLRLSCAASGFSFNTYTMHWVRQAPGKGLEWVADIAYDGSTKYYADSVKGRFTISRDNAKNSLYLQMNSLRAEDTAVYYCARDAVAGEGYFDLWGRGTLVTVSS

SEQ ID NO: 6 - protein, 110 aa, Artificial sequence
  Synthetic F12 light chain
QSALTQDPAVSVALGQTVRITCQGDSLRSYYASWYQQLPGTAPKLLIYRNNQRPSGVPDRFSGSKSGTSASLAISGLRSEDEADYYCAAWDDSLSAWVFGGGTKLTVLGA

SEQ ID NO: 7 - protein, 245 aa, Artificial sequence
  Synthetic F12
QVQLQESGGGLVQPGGSLRLSCAASGFSFNTYTMHWVRQAPGKGLEWVADIAYDGSTKYYADSVKGRFTISRDNAKNSLYLQMNSLRAEDTAVYYCARDAVAGEGYFDLWGRGTLVTVSSGGGGSGGGGSGGGGSQSALTQDPAVSVALGQTVRITCQGDSLRSYYASWYQQLPGTAPKLLIYRNNQRPSGVPDRFSGSKSGTSASLAISGLRSEDEADYYCAAWDDSLSAWVFGGGTKLTVLGA

SEQ ID NO: 8 - protein, 118 aa, Artificial sequence
  Synthetic C32 heavy chain
VQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKVSSSWSHFDYWGQGTLVTVSS

SEQ ID NO: 9 - protein, 109 aa, Artificial sequence
  Synthetic C32 light chain
DVVMTQSPSTLSASVGDRVTITCRASQYISNWLAWYQQKPGKAPKLLIYKASSLESGVPSRFSGSGSGTEFTLTISSLQPEDFATYYCQESYNTPLFTFGPGTKLEIKR

SEQ ID NO: 10 - protein, 243 aa, Artificial sequence
  Synthetic C32
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKVSSSWSHFDYWGQGTLVTVSSGGGGSGGGGSGGGGSDVVMTQSPSTLSASVGDRVTITCRASQYISNWLAWYQQKPGKAPKLLIYKASSLESGVPSRFSGSGSGTEFTLTISSLQPEDFATYYCQESYNTPLFTFGPGTKLEIKR

SEQ ID NO: 11 - protein, 5 aa, Artificial sequence
  Synthetic C32 CDR1-H (Kabat)
SYAMS

SEQ ID NO: 12 - protein, 16 aa, Artificial sequence
  Synthetic C32 CDR2-H (Kabat)
AISGSGGSTYYADSVK

SEQ ID NO: 13 - protein, 10 aa, Artificial sequence
  Synthetic C32 CDR3-H (Kabat)
VSSSWSHFDY

SEQ ID NO: 14 - protein, 11 aa, Artificial sequence
  Synthetic C32 CDR1-L (Kabat)
RASQYISNWLA

SEQ ID NO: 15 - protein, 7 aa, Artificial sequence
  Synthetic C32 CDR2-L (kabat)
KASSLES

SEQ ID NO: 16 - protein, 10 aa, Artificial sequence
  Synthetic C32 CDR3-L
QESYNTPLFT

SEQ ID NO: 17 - protein, 8 aa, Artificial sequence
  Synthetic C32 CDR1-H (IMGT)
GFTFSSYA

SEQ ID NO: 18 - protein, 8 aa, Artificial sequence
  Synthetic C32 CDR2-H (IMGT)
ISGSGGST

SEQ ID NO: 19 - protein, 12 aa, Artificial sequence
  Synthetic C32 CDR3-H (IMGT)
AKVSSSWSHFDY

SEQ ID NO: 20 - protein, 6 aa, Artificial sequence
  Synthetic C32 CDR1-L (IMGT)
QYISNW

SEQ ID NO: 21 - protein, 3 aa, Artificial sequence
  Synthetic C32 CDR2-L (IMGT)
KAS

SEQ ID NO: 22 - protein, 10 aa, Artificial sequence
  Synthetic C32 CDR3-L
QESYNTPLFT

SEQ ID NO: 23 - protein, 119 aa, Artificial sequence
  Synthetic F2 heavy chain
QVQLQQSGGGVVQPGGSLRLSCAASEFTFSASGMHWVRQAPGKGLEWMAFIAYDGNQKFYADSVKGRFTISRDNSKNTLYLQMDSLRGEDTAVYYCAKEMQREGYFDYWGQGTLVTVSS

SEQ ID NO: 24 - protein, 109 aa, Artificial sequence
  Synthetic F2 light chain
NFMLTQDPAVSVALGQTVRITCQGDSLRSYYASWYQQKPGQAPVLVIYGKNNRPSGIPDRFSGSKSGNSASLDISGLQSEDEADYYCATWDDNLSGPIFGGGTKVTVLG

SEQ ID NO: 25 - protein, 243 aa, Artificial sequence
  Synthetic F2
QVQLQQSGGGVVQPGGSLRLSCAASEFTFSASGMHWVRQAPGKGLEWMAFIAYDGNQKFYADSVKGRFTISRDNSKNTLYLQMDSLRGEDTAVYYCAKEMQREGYFDYWGQGTLVTVSSGGGGSGGGGSGGGGSNFMLTQDPAVSVALGQTVRITCQGDSLRSYYASWYQQKPGQAPVLVIYGKNNRPSGIPDRFSGSKSGNSASLDISGLQSEDEADYYCATWDDNLSGPIFGGGTKVTVLG

SEQ ID NO: 26 - protein, 5 aa, Artificial sequence
  Synthetic F2 CDR1-H
ASGMH

SEQ ID NO: 27 - protein, 17 aa, Artificial sequence
  Synthetic F2 CDR2-H
FIAYDGNQKFYADSVKG

SEQ ID NO: 28 - protein, 10 aa, Artificial sequence
  Synthetic F2 CDR3-H
EMQREGYFDY

SEQ ID NO: 29 - protein, 11 aa, Artificial sequence
  Synthetic F2 CDR1-L
QGDSLRSYYAS

SEQ ID NO: 30 - protein, 7 aa, Artificial sequence
  Synthetic F2 CDR2-L
GKNNRPS

SEQ ID NO: 31 - protein, 11 aa, Artificial sequence
  Synthetic F2 CDR3-L
ATWDDNLSGPI

SEQ ID NO: 32 - protein, 8 aa, Artificial sequence
  Synthetic F2 CDR1-H (IMGT)
EFTFSASG

SEQ ID NO: 33 - protein, 8 aa, Artificial sequence
  Synthetic F2 CDR2-H ( IMGT)
IAYDGNQK

SEQ ID NO: 34 - protein, 12 aa, Artificial sequence
  Synthetic F2 CDR3-H
AKEMQREGYFDY

SEQ ID NO: 35 - protein, 6 aa, Artificial sequence
  Synthetic F2 CDR1-L (IMGT)
SLRSYY

SEQ ID NO: 36 - protein, 3 aa, Artificial sequence
  Synthetic F2 CDR2-L
GKN

SEQ ID NO: 37 - protein, 11 aa, Artificial sequence
  Synthetic F2 CDR3-L (IMGT)
ATWDDNLSGPI

SEQ ID NO: 38 - protein, 119 aa, Artificial sequence
  Synthetic H9 heavy chain
QVQLAESGGGLVKPGGSLRLSCAASGFTFSDYYMSWIRQAPGKGLEWVSYISTSGSSIYYVDSVKGRFTISRDNAKNSLYLQMDSLRDDDTAVYYCARDLHGDYAFDSWGQGTLVTVSS

SEQ ID NO: 39 - protein, 108 aa, Artificial sequence
  Synthetic H9 light chain
SELTQDPAVSVALGQTVRITCQGDSLRSYYASWYQQKPGQAPVLVIYGKNNRPSGIPDRFSGSKSGNSASLDISGLQSEDEADYYCATWDDNLSGPIFGGGTKVTVLG

SEQ ID NO: 40 - protein, 242 aa, Artificial sequence
  Synthetic H9
QVQLAESGGGLVKPGGSLRLSCAASGFTFSDYYMSWIRQAPGKGLEWVSYISTSGSSIYYVDSVKGRFTISRDNAKNSLYLQMDSLRDDDTAVYYCARDLHGDYAFDSWGQGTLVTVSSGGGGSGGGGSGGGGSSELTQDPAVSVALGQTVRITCQGDSLRSYYASWYQQKPGQAPVLVIYGKNNRPSGIPDRFSGSKSGNSASLDISGLQSEDEADYYCATWDDNLSGPIFGGGTKVTVLG

SEQ ID NO: 41 - protein, 5 aa, Artificial sequence
  Synthetic H9 CDR1-H (Kabat)
DYYMS

SEQ ID NO: 42 - protein, 17 aa, Artificial sequence
  Synthetic H9 CDR2-H (Kabat)
YISTSGSSIYYVDSVKG

SEQ ID NO: 43 - protein, 10 aa, Artificial sequence
  Synthetic H9 CDR3-H
DLHGDYAFDS

SEQ ID NO: 44 - protein, 11 aa, Artificial sequence
  Synthetic H9 CDR1-L
QGDSLRSYYAS

SEQ ID NO: 45 - protein, 7 aa, Artificial sequence
  Synthetic H9 CDR2-L
GKNNRPS

SEQ ID NO: 46 - protein, 11 aa, Artificial sequence
  Synthetic H9 CDR3-L
ATWDDNLSGPI

SEQ ID NO: 47 - protein, 8 aa, Artificial sequence
  Synthetic H9 CDR1-H (IMGT)
GFTFSDYY

SEQ ID NO: 48 - protein, 8 aa, Artificial sequence
  Synthetic H9 CDR2-H (IMGT)
ISTSGSSI

SEQ ID NO: 49 - protein, 12 aa, Artificial sequence
  Synthetic H9 CDR3-H
ARDLHGDYAFDS

SEQ ID NO: 50 - protein, 6 aa, Artificial sequence
  Synthetic H9 CDR1-L (IMGT)
SLRSYY

SEQ ID NO: 51 - protein, 3 aa, Artificial sequence
  Synthetic H9 CDR2-L (IMGT)
GKN

SEQ ID NO: 52 - protein, 11 aa, Artificial sequence
  Synthetic H9 CDR3-L (IMGT)
ATWDDNLSGPI

SEQ ID NO: 53 - protein, 118 aa, Artificial sequence
  Synthetic G9 heavy chain
QVQLVESGGGLVEPGGSLRLSCAASGFTFSNYAINWVRQAPGKGLEWVANIHHDGNGKYYVDSVEGRFTISRDNAKNSLYLQMDSLRAEDTAIYYCARDGYGGYLDLWGQGTLVTVSS

SEQ ID NO: 54 - protein, 108 aa, Artificial sequence
  Synthetic G9 light chain
SELTQDPAVSVALGQTVRITCQGDSLRSYYASWYQQKPGQAPVLVIYGKNNRPSGIPDRFSGSGSGNTASLTITGAQAEDEADYYCAAWDDSLSGPVFGGGTKVTVLG

SEQ ID NO: 55 - protein, 241 aa, Artificial sequence
  Synthetic G9
QVQLVESGGGLVEPGGSLRLSCAASGFTFSNYAINWVRQAPGKGLEWVANIHHDGNGKYYVDSVEGRFTISRDNAKNSLYLQMDSLRAEDTAIYYCARDGYGGYLDLWGQGTLVTVSSGGGGSGGGGSGGGGSSELTQDPAVSVALGQTVRITCQGDSLRSYYASWYQQKPGQAPVLVIYGKNNRPSGIPDRFSGSGSGNTASLTITGAQAEDEADYYCAAWDDSLSGPVFGGGTKVTVLG

SEQ ID NO: 56 - protein, 5 aa, Artificial sequence
  Synthetic G9 CDR1-H (Kabat)
NYAIN

SEQ ID NO: 57 - protein, 17 aa, Artificial sequence
  Synthetic G9 CDR2-H (Kabat)
NIHHDGNGKYYVDSVEG

SEQ ID NO: 58 - protein, 9 aa, Artificial sequence
  Synthetic G9 CDR3-H (kabat)
DGYGGYLDL

SEQ ID NO: 59 - protein, 11 aa, Artificial sequence
  Synthetic G9 CDR1-L (Kabat)
QGDSLRSYYAS

SEQ ID NO: 60 - protein, 7 aa, Artificial sequence
  Synthetic G9 CDR2-L (Kabat)
GKNNRPS

SEQ ID NO: 61 - protein, 11 aa, Artificial sequence
  Synthetic G9 CDR3-L
AAWDDSLSGPV

SEQ ID NO: 62 - protein, 8 aa, Artificial sequence
  Synthetic G9 CDR1-H -IMGT)
GFTFSNYA

SEQ ID NO: 63 - protein, 8 aa, Artificial sequence
  Synthetic G9 CDR2-H (IMGT)
IHHDGNGK

SEQ ID NO: 64 - protein, 11 aa, Artificial sequence
  Synthetic G9 CDR3-H (IMGT)
ARDGYGGYLDL

SEQ ID NO: 65 - protein, 6 aa, Artificial sequence
  Synthetic G9 CDR1-L (IMGT)
SLRSYY

SEQ ID NO: 66 - protein, 3 aa, Artificial sequence
  Synthetic G9 CDR2-L (IMGT)
GKN

SEQ ID NO: 67 - protein, 11 aa, Artificial sequence
  Synthetic G9 CDR3-L (IMGT)
AAWDDSLSGPV

SEQ ID NO: 68 - protein, 5 aa, Artificial sequence
  Synthetic H7 CDR1-H (Kabat)
SYAMH

SEQ ID NO: 69 - protein, 17 aa, Artificial sequence
  Synthetic H7 CDR2-H (Kabat)
VISYDGSNKYYADSVKG

SEQ ID NO: 70 - protein, 11 aa, Artificial sequence
  Synthetic H7 CDR3-H (Kabat)
DLSGYGDYPDY

SEQ ID NO: 71 - protein, 11 aa, Artificial sequence
  Synthetic H7 CDR1-L (Kabat)
QGDSLRSYYAS

SEQ ID NO: 72 - protein, 7 aa, Artificial sequence
  Synthetic H7 CDR2-L (Kabat)
GRNERPS

SEQ ID NO: 73 - protein, 11 aa, Artificial sequence
  Synthetic H7 CDR3-L (Kabat)
AGWDDSLTGPV

SEQ ID NO: 74 - protein, 8 aa, Artificial sequence
  Synthetic H7 CDR1-H (IMGT)
RFTFSSYA

SEQ ID NO: 75 - protein, 7 aa, Artificial sequence
  Synthetic H7 CDR2-H 5IMGT)
ISYDGSN

SEQ ID NO: 76 - protein, 13 aa, Artificial sequence
  Synthetic H7 CDR-3 H (IMGT)
ARDLSGYGDYPDY

SEQ ID NO: 77 - protein, 6 aa, Artificial sequence
  Synthetic H7 CDR1-L (IMGT)
SLRSYY

SEQ ID NO: 78 - protein, 3 aa, Artificial sequence
  Synthetic H7 CDR2-L (IMGT)
GRN

SEQ ID NO: 79 - protein, 11 aa, Artificial sequence
  Synthetic H7 CDR3-L (IMGT)
AGWDDSLTGPV

SEQ ID NO: 80 - protein, 5 aa, Artificial sequence
  Synthetic F12 CDR1-H (Kabat)
TYTMH

SEQ ID NO: 81 - protein, 17 aa, Artificial sequence
  Synthetic F12 CDR2-H (Kabat)
DIAYDGSTKYYADSVKG

SEQ ID NO: 82 - protein, 11 aa, Artificial sequence
  Synthetic F12 CDR3-H (Kabat)
DAVAGEGYFDL

SEQ ID NO: 83 - protein, 11 aa, Artificial sequence
  Synthetic F12 CDR1-L (Kabat)
QGDSLRSYYAS

SEQ ID NO: 84 - protein, 7 aa, Artificial sequence
  Synthetic F12 CDR2-L (Kabat)
RNNQRPS

SEQ ID NO: 85 - protein, 11 aa, Artificial sequence
  Synthetic F12 CDR3- L (Kabat)
AAWDDSLSAWV

SEQ ID NO: 86 - protein, 8 aa, Artificial sequence
  Synthetic F12 CDR1-H (IMGT)
GFSFNTYT

SEQ ID NO: 87 - protein, 8 aa, Artificial sequence
  Synthetic F12 CDR2-H (IMGT)
IAYDGSTK

SEQ ID NO: 88 - protein, 13 aa, Artificial sequence
  Synthetic H12 CDR3-H
ARDAVAGEGYFDL

SEQ ID NO: 89 - protein, 6 aa, Artificial sequence
  Synthetic H12 CDR1-L (IMGT)
SLRSYY

SEQ ID NO: 90 - protein, 3 aa, Artificial sequence
  Synthetic H12 CDR2-L (IMGT)
RNN

SEQ ID NO: 91 - protein, 11 aa, Artificial sequence
  Synthetic H12 CDR3-L (IMGT)
AAWDDSLSAWV

SEQ ID NO: 92 - protein, 107 aa, Artificial sequence
  Synthetic VH4 light chain
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 93 - protein, 122 aa, Artificial sequence
  Synthetic VH4 heavy chain
EVQLVQSGGGVVQPGESLRLSCAASGFTFSSYSMNWVRQAPGKGLEWVSVIYSGGSTYYADSVKGRFTISRDNSKNTLYLQMNSLRAEDTAVYYCAREVYHSSGYDDAFDIWGRGTMVTVSS

SEQ ID NO: 94 - protein, 5 aa, Artificial sequence
  Synthetic VH4 CDR1-H (Kabat)
SYSMN

SEQ ID NO: 95 - protein, 16 aa, Artificial sequence
  Synthetic VH4 CDR2-H (Kabat)
VIYSGGSTYYADSVKG

SEQ ID NO: 96 - protein, 12 aa, Artificial sequence
  Synthetic VH4 CDR3-H (Kabat)
YHSSGYDDAFDI

SEQ ID NO: 97 - protein, 11 aa, Artificial sequence
  Synthetic VHA CDR1-L (Kabat)
RASEGIYHWLA

SEQ ID NO: 98 - protein, 7 aa, Artificial sequence
  Synthetic VH4 CDR2-L (Kabat)
KASSLAS

SEQ ID NO: 99 - protein, 9 aa, Artificial sequence
  Synthetic VH4 CDR3-L
QQYSNYPLT

SEQ ID NO: 100 - protein, 8 aa, Artificial sequence
  Synthetic VH4 CDR1-H (IMGT)
GFTFSSYS

SEQ ID NO: 101 - protein, 7 aa, Artificial sequence
  Synthetic VH4 CDR2-H (IMGT)
IYSGGST

SEQ ID NO: 102 - protein, 14 aa, Artificial sequence
  Synthetic VH4 CDR3-H
EVYHSSGYDDAFDI

SEQ ID NO: 103 - protein, 6 aa, Artificial sequence
  Synthetic VH4 CDR1-L (IMGT)
EGIYHW

SEQ ID NO: 104 - protein, 3 aa, Artificial sequence
  Synthetic VH4 CDR2-L
KAS

SEQ ID NO: 105 - protein, 9 aa, Artificial sequence
  Synthetic VH4 CDR3-L (IMGT)
QQYSNYPLT

(19) United States
(12) Patent Application Publication
(10) Pub. No.: US 2023/0183379 A1
(43) Pub. Date: Jun. 15, 2023

(54) BISPECIFIC ANTIBODY TARGETING TRANSFERRIN RECEPTOR 1 AND SOLUBLE ANTIGEN

(71) Applicants: INSERM (INSTITUT NATIONAL DE LA SANTÉ ET DE LA RECHERCHE MÉDICALE), Paris (FR); INSTITUT REGIONAL DU CANCER DE MONTPELLIER, Montpellier (FR); UNIVERSITE DE MONTPELLIER, Montpellier (FR)

(72) Inventors: Marie-Alix POUL, Montpellier Cedex 5 (FR); Adrien LAROCHE, Montpellier Cedex 5 (FR)

(21) Appl. No.: 17/295,593

(22) PCT Filed: Nov. 20, 2019
(86) PCT No.: PCT/EP2019/081873

§ 371 Date: May 20, 2021

(30) Foreign Application Priority Data
Nov. 20, 2018 (EP) 18306524.2

(51) Int. Cl. C07K 16/46; A61P 35/00

(52) U.S. Cl. CPC C07K 16/468; A61P 35/00; C07K 2317/77; C07K 2317/31

(57) ABSTRACT

The invention relates to a bispecific antibody targeting TfR1 and a soluble antigen. The inventors demonstrate that the unique mode of interaction of the bispecific antibody with TfR1 increases its persistence in vivo through an FcRn-like mechanism. It has been demonstrated on MCF7 cell line that the bispecific antibody induces soluble antigen (IL6) uptake through TfR1 mediated endocytosis. Effects of the bispecific antibody on XG6 cell lines viability have been demonstrated, notably on iron and IL-6 deprivation. Hence, the inventors design an improved sweeping antibody which can specifically target tumors and inflammatory cells expressing TfR1. By its unique mode of interaction with TfR1, its ability to induce soluble uptake antigen through TfR1 mediated endocytosis and its capacity to deprive cells of iron, known for being required in tumors growth and progression and development of inflammatory pathologies, the bispecific antibody can be used in the treatment of cancer and inflammatory pathologies.

### TECHNICAL FIELD

The invention pertains to the field of bispecific antibody and its use in the treatment of cancer and inflammatory pathologies.

### BACKGROUND ART

Monoclonal antibodies have become a general modality in therapeutic development, and a variety of monoclonal antibodies targeting soluble antigens have been developed. However, even with infinite binding affinity to an antigen, a conventional antibody can bind to the antigen only once and results in an increase in total plasma antigen concentration in vivo. This antibody-mediated antigen accumulation generally occurs because the clearance from circulation of an antibody-antigen complex is much slower than that of a free antigen. This limitation has recently been overcome by sweeping antibodies, which are capable of actively eliminating soluble antigens from circulation.

Sweeping antibody is described in Igawa et al., “Sweeping antibody as novel therapeutic modality”, 2016 John Wiley & Sons A/S. Published by John Wiley & Sons Ltd, Immunological Reviews 270/2016 and in the patent application EP2752200. A sweeping antibody incorporates two antibody engineering technologies: one is variable region engineering to enable the antibody to bind to an antigen in plasma and dissociate from the antigen in endosome (after which the antigen undergoes lysosomal degradation), and the other is constant region engineering to increase the cellular uptake of the antibody-antigen complex into endosome. By enhancing the elimination of soluble antigens from circulation, sweeping antibodies can therapeutically target soluble antigens that conventional antibodies cannot. More particularly, and according to Igawa et al. the novel antibody termed sweeping antibody incorporates the ability to bind an antigen pH-dependently and also uses the Fc receptor to accelerate the uptake of antibody-antigen complexes. Igawa et al. describes that two Fc receptors, FcRn and FcγRIIb could be exploited as receptors for a sweeping antibody.

Potential target and therapeutic application are described for sweeping antibodies such as cancer treatment like glioblastoma, chronic diseases, for example rheumatoid arthritis and asthma or Alzheimer's disease.

It is however necessary to improve the treatment of these pathologies. The inventors hence design an improved sweeping antibody which can specifically target specific cells such as cancerous cells or inflammatory cells in order to develop targeted therapies.

### SUMMARY OF THE INVENTION

During the iron intake process in generic cells, transferrin receptors (TfRs) act as the most important receptor mediated controls. TfR1 and TfR2 are two subtypes of TfRs that bind with iron-transferrin complex to facilitate iron into cells. TfR1 is ubiquitously expressed on the surfaces of generic cells, whereas TfR2 is specially expressed in liver cells.

Transferrin (Tf) is a serum protein of 80 kDa, the role of which is to fix soluble iron. Iron charged transferin (holo-Tf) is endocyted in cells due to its binding with the transferrin receptor 1 (TfR1) at physiological extracellular pH. Acidification of the endosome causes a conformational change which salts out the iron in the cytosol. Upon its natural ligand holo-Tf binding, TfR1 is rapidly internalized and recycled after holo-Tf has released iron in the endosomes. Because apo-Tf (apo-Tf corresponds to Tf not saturated by iron) is still tightly bound to TfR1 under acidic conditions of the endosome, the apo-Tf/TfR complex is then re-exported to the membrane, where the return to a physiological pH causes a dissociation of the apo-Tf/TfR complex.

The inventors discovered that anti-TfR1 antibodies that bind with high affinity to TfR1 act like an exact mimic of the natural ligand. Furthermore, these anti-TfR1 antibodies bind with similar affinity to TfR1 at extracellular and endosomal pH. Hence, anti-TfR1 antibodies are recycled at the cell surface with the receptor TfR1 after it has induced its internalization, thus immediately preventing TfR1 association with extracellular holo-Tf. Furthermore, the anti-TfR1 antibody specificity and their unique mode of interaction with TfR1 increase their persistence in vivo through an FcRn-like mechanism that is independent of the Fc part of the antibody.

Hence, the present invention concerns a bispecific antibody comprising a first antigen-binding domain that competitively binds to the transferrin receptor 1 (TfR1) on the surface of a target cell and a second antigen-binding domain which binds to a soluble antigen wherein the binding activity is a calcium-dependent antigen binding and/or a pH-dependent antigen binding activity.

The bispecific antibody according to the present invention recognizes the endocytable cell surface target TfR1 and simultaneously binds to a soluble antigen. Hence, and advantageously, the soluble antigen bound to the second antigen-binding domain of the bispecific antibody can be actively taken up into the cell via TfR1 mediated endocytosis and because the second antigen-binding domain as a pH- or a calcium-dependent antigen-binding property, is dissociated in endosome.

As the first antigen-binding domain binds with high affinity to TfR1 and does not dissociate at acidic pH, the first antigen-binding domain is recycled at the cell surface with the receptor TfR1. Furthermore, this specific binding inhibits the binding of transferrin to the transferrin receptor and which therefore deprive cells of iron.

Thus, the bispecific antibody of the present invention presents the double advantage to significantly accelerate the clearance of soluble antigen from the circulation and/or from the extracellular medium by its ability to induce soluble antigen uptake through TfR1 mediated endocytosis and to deprive cells of iron, known for being required in tumors (including tumor cells, cancer stem cells and cells from the tumor microenvironment) growth and progression.

As tumors cells overexpress TfR1 and have increased iron needs, the present invention also concerns the use of bispecific antibody according to the present invention for treating cancers.

Furthermore, inflammatory cells and notably macrophages also have transferrin receptor 1 (TfR1). The present invention thus also relates to bispecific antibody for treating inflammatory pathologies.

Hence, bispecific antibodies according to the present invention advantageously target tumoral and inflammatory cells.

### BRIEF DESCRIPTION OF DRAWINGS

Other features, details and advantages will be shown in the following detailed description and on the figures, on which:

FIG. 1 concerns preliminary characterization of the reformatted anti-TfR1 scFv antibodies;

FIG. 1A represents the graphic representation of the (scFv)₂-Fc and the IgG1 formats, in grey variable domains (light grey, VH; dark grey, VL), in black, constant domains.

FIG. 1B represents TfR1 surface expression at the surface of Raji cells (human lymphoma) and P815 mastocytoma cells (mouse) by FACS (FC500 cytometer) with a commercial mouse anti-human TfR1 IgG or rat anti-mouse TfR1 IgG (10 μg/mL), followed by anti-mouse IgG or anti-rat IgG fluorescent secondary antibodies, respectively, or with Alexa 488-conjugated holo-Tf (500 nM).

FIG. 1C Detection of the binding of the anti-TfR1 antibodies reformatted into bivalent scFv by fusion to Fc (upper panels) or into full length human IgG1 (lower panels to the Raji or the mouse P815 cell lines, as indicated. Binding is detected with an anti-human IgG1 antibody conjugated to FITC and FACS analysis (FS500 cytometer). Dark grey peaks represent fluorescent background of the secondary antibody alone or, in case of the detection of fluorescent holo-Tf binding, cell autofluorescence.

FIG. 1D scFv₂-Fc (left panel) and full length IgG1 (right panel) interference with fluorescent holo-Tf internalization in Raji cells: antibodies at the indicated concentrations are combined with fluorescent holo-Tf (500 nM) and incubated at 37° C. with Raji cells for 3 h then cells are collected, washed with PBS and analyzed by FACS. Results are expressed in Mean Fluorescent Intensity (MFI) relative to cells incubated with fluorescent holo-Tf only. Irr, irrelevant antibody of the same format. The data shown are representative of 3 independent experiments.

FIG. 2 Setting up of the holo-Tf cell internalization test

FIG. 2A, 2B Raji cells were incubated at 37° C. or 4° C. (to allow or not internalization, respectively) with 500 nM holo-Tf conjugated to Alexa-488 (holo-Tf-A488) in culture medium for the indicated times. Cells were then washed with PBS, incubated or not with NaCl-glycine buffer (50 mM glycine pH 2.8, 500 mM NaCl) at 4° C. for 10 min, to remove surface-bound holo-Tf-A488, then washed again with PBS, and cell fluorescence was measured by FACS. Total, fluorescence in cells without glycine step; intracellular, fluorescence in cells with glycine step. Fluorescence increased from 1 to 3 h and was more than 95% intracellular; therefore, the glycine incubation step was omitted in further experiments (i.e. in FIG. 1D). The cell mean fluorescence intensity (MFI) was calculated using the Flow Jo Version 10.1r7 software.

FIG. 2C Raji cells were incubated with 500 nM holo-Tf-A488 together with increasing concentrations of unconjugated holo-Tf at 37° C. for 3 h. Increasing concentrations of unconjugated holo-Tf reduced fluorescence accumulation (IC50=580 nM) in a dose-dependent manner, showing the specificity of the holo-Tf internalization test used in FIG. 1D.).

FIG. 3 Characterization of the anti-TfR1 H7 scFv₂-Fc and full length IgG1 antibodies;

FIG. 3A Interference of H7-Fc and H7-IgG1 (left and right panel, respectively) with the internalization of 10 μM or 1 μM Alexa 488-conjugated holo-Tf, measured as in FIG. 1C.

FIG. 3B Apparent affinity of H7-Fc, H7-IgG1 and Ba120 (mouse monoclonal anti-TfR1 IgG1) and of Alexa 488-conjugated holo-Tf measured by detection of the binding of increasing concentrations of antibody/holo-Tf in Raji cells at 4° C. Bound antibodies were detected with a mouse anti-human-Fc fluorescent antibody and analyzed by FACS; results are expressed as MFI in function of the primary antibody concentration. The EC50 values (nM) are indicated.

FIG. 3C Measurement of the fluorescence signal in Raji cells after incubation (at 4° C. for 1 h) with 500 nM Alexa 488-conjugated holo-Tf and increasing concentrations of H7-Fc, H7-IgG1, irrelevant scFv₂-Fc antibody, or Ba120. Results are expressed as the % MFI compared with cells incubated with holo-Tf alone.

FIG. 3D Apparent affinity of H7-Fc and H7-IgG1 for mouse TfR1 measured by detection of the binding of increasing concentrations of antibody in P815 cells at 4° C. as in B]

FIG. 3E H7-Fc (left panel) and Ba120 (right panel) binding to TfR1 in Raji cells in the presence of increasing concentrations of holo-Tf. Bound antibodies (1 nM) were detected with anti-human-Fc or anti-mouse-Fc fluorescent secondary antibodies, and results expressed as MFI. The IC₅₀ values (nM) are indicated. Data are representative of 2-3 independent experiments.

FIG. 4 represents the comparison of TfR1 (or HIF-1α) total level after treatment of Raji cells with H7-IgG1, Ba120 and human-holo-Tf in different conditions.

FIG. 4A represents effect of incubation with H7-IgG1 (5 μg/mL), holo-Tf (10 μM), Ba120 or irrelevant IgG1 antibody (Irr.) (5 μg/mL) (for 1.5 day) on TfR1 and HIF-1αlevels in Raji cells. Protein extracts (20 μg) were separated by SDS-PAGE (7% polyacrylamide separation gels) and analyzed by western blotting. Data are representative of 3 independent experiments.

FIG. 4B), Raji cells were treated with antibodies or holo-Tf as in A. for 18 hours in the presence of 10 mM NH₄Cl (to blocks lysosomal degradation) or 50 μg/mL cycloheximide (CHX) (to block translation) as indicated or not, and TfR1 levels were quantified like in FIG. 4A.

FIG. 5 represents comparison of H7-IgG1, Ba120 and human-holo-Tf binding to native human TfR1 at different pH values.

FIG. 5A describes the protocol used to study H7-IgG1 binding to TfR1 at different pH values, similar to those encountered during physiological TfR1 internalization and recycling after holo-Tf binding. The experiment was performed at 4° C. Raji cells were incubated with anti-TfR1 antibodies (10 μg/mL) or human holo-Tf conjugated to Alexa488 (500 nM) at pH 7 for 1 h. Unbound antibodies or holo-Tf were eliminated by washing at pH 7, and then cells were incubated at a given pH (from 7 to 5), to mimic the conditions within endosomes after internalization (1 h at 4° C.). A final wash was performed at pH 7, to mimic the conditions after TfR1 recycling at the cell surface. FITC-conjugated anti-human or mouse IgG secondary antibodies were used to detect by FACS the remaining bound antibodies, H7-IgG1 or Ba120, respectively, after these steps.

FIG. 5B describes results which are expressed as the percentage of the Mean Fluorescent Intensity (MFI) relative to the MFI of cells kept at pH 7 for the entire experiment. As expected, holo-Tf binding decreased at lower pH, because of the loss of Fe at low pH and the reduced affinity of apo-Tf at pH 7.

FIG. 5C The iron content of holo-Tf at various pH values was monitored by taking advantage of the fact that holo-Tf, but not apo-Tf, displays an absorption peak at 460 nm.14 Holo-Tf (10 μM) was resuspended in buffer at various pH. Results are expressed as the ratio of the sample absorbance at 460 nm normalized to the standard protein concentration obtained at 280 nm.

FIG. 6 schematizes a tetravalent bispecific antibody BsAb anti-TfR1-anti-IL-6 with pH dependent binding.

FIG. 7 schematizes a bispecific antibody devoided of Fc region with monovalent or bivalent binding to TfR1 and monovalent pH-dependent binding to IL-6.

FIG. 8 represents the binding of the BsAb on TfR1 on RAJI cells (Burkitt's lymphoma) and TfR1 modulation by BsAb on BxPC3 cells.

FIG. 8A Cells are washed 2 times with PBS 1% FCS, cells are incubated with BsAb (of FIG. 7A) or IgG1 anti-TfR1 F12 antibody during 1 h a 4° C. in PBS 1% FCS. Concentrations start from 100 nM to 0,0001 nM. Cells are washed twice with PBS 1% FCS and incubated with an anti-human IgG (Fc specific)-FITC (1/100) antibody (F9512 Sigma) during 1 h a 4° C. in PBS 1% FCS before FACS analysis. EC₅₀ (nM) are indicated.

FIGS. 8B and 8C BxPC3 cells are plated in RPMI 5% FCS a day before treatment for 48 h with F12 or BsAb to (39 nM each ie 5 μg/mL and 10 μg/mL, respectively). [FIG. 8B] For Western blot, proteins are extracted with KOOK buffer and 50 g of protein are used to measure TfR1 levels. Primary antibodies (1/1000): Transferrin Receptor Mouse Monoclonal Antibody (H68.4), Catalog #13-6800. [FIG. 8C] Secondary antibodies (1/2500): Anti Mouse IgG Sigma A3673© For FACS, cells are collected and TfR1 expression is measured with an APC conjugated Mouse Anti-Human CD71 Clone M-A712 which does not compete with F12 antibody recognition site on TfR1.

FIG. 9 describes the measurement of pH dependent binding of BsAb to IL-6 by sandwich ELISA. A capture antibody (1 μg/mL) is prepared the day before on a MAXISORP ELISA plate. The next day, washes with PBS tween 20 0.05%, blocking with PBS 1% BSA during 2 hrs. Incubation of IL6 (10 ng/mL) for 2 hours at Room Temperature (RT). Antibodies are diluted in Na₂HPO₄/NaH₂PO₄ buffers adjusted at pH 7.4, 6.8 or 6.0. Washes are also done with pH adjusted buffers. Incubation (39 nM) BsAb or anti-IL6 VH4 10 mAb (FIG. 9A) or anti-IL6 BE8 mAb (FIG. 9B) during 2 h at Room Temperature (RT). Washes are done again with pH adjusted buffers. Secondary antibodies A7164 diluted (1/1000) in PBS for 50 min at RT. Washings and Revelation with 100 μL TMB for 20 min well addition of 100 μL of H₂SO₄ 2M. OD measurement at 450 nm.

FIG. 10 Detection of endogenous IL-6 by the bi-specific antibody. The ability of the BsAb to bind to endogenous IL-6 and native TfR1 at the same time was visualized by immunofluorescence. For each assay, 2.10⁴ IL-6 producing pancreatic cancer CFPAC cells were plated on cover slips in a 24-well plate (complete IMDM medium). Two days later, during the logarithmic phase of growth, the cells were incubated with 30 μg/mL antibodies (FIG. 10 A): VH4 (anti-IL-6 Human IgG1), F12 (anti TfR1 Human IgG1), Human Bispecific anti IL6/TfR1, a combination of VH4 and F12, or no Ab, as indicated, and placed at 4° C. for 90 min. Cells were washed twice with PBS-BSA (1 mg/ml) and once with PBS. Cells were fixed and permeabilized using formalin (3.7% p-formaldehyde in PBS) 40 min. washed twice with PBS-BSA and saturated for 45 min in PBS-BSA. The cells were 25 incubated with an anti Human IgG FITC during 90 min., washed three times with PBS (DAPI was included in the final wash to stain DNA) and were then prepared for fluorescent microscopic visualization by Everbrithe® with DAPI and visualized using an epifluorescence Zeiss Imager 2. In (FIG. 10B), to be able to colocalize IL-6 and TfR1, CFPAC cells incubated with a mix of the BsAb and anti-IL6 BE8 mouse IgG (that do not compete with VH4 for IL-6 binding) (30 each g/mL) (upper panel) or with a mix of anti-TfR1 F12 and the anti-IL6 BE8 antibody (lower panel) were further fixed, permeabilized and co-stained with anti-hu-Fc-FITC and anti-Mouse-Fc-PE secondary antibodies. DAPI was included in the final wash to stain DNA.

FIG. 11 Pancreatic cancer cell lines CFPAC, HPAC and BxPC3 were assayed for (FIG. 11A) IL-6 and IL-6R expression by RT-qPCR, for (FIG. 11B) detection of IL-6 in HPAC and CFPAC culture supernatant after 2 days of culture (ELISA), and for (FIG. 11C) detection of IL6 by Immunofluorescence using the anti-IL6 BE8 mouse antibody. Dapi was used for nuclear counter staining. Only CFPAC express IL-6 at detectable concentration by ELISA and immunofluorescence. (FIG. 11D) XG6 and (FIG. 12E) XG7 myeloma cell lines were incubated in IMDM 10% FSC with increasing concentration of IL-6 (left) or with IMDM 10% FSC 40 pM IL6 and increasing concentrations of the neutralizing anti-IL6 antibody BE8, as indicated. Viability was measured after 3 days using the Cell titer Glow. Both myeloma cell lines are IL-6 dependent.

The XG6 and XG7 myeloma cell lines were dependent of IL-6 for their growth (FIG. 11D) as previously published. The EC₅₀ (concentration of IL-6 allowing 50% of maximum growth) was determined to be around 0.4 pM for both cell lines. When cells were grown in the presence of 40 pM of IL-6, the neutralizing anti-IL-6 BE8 inhibited XG6 and XG7 growth (IC₅₀ of 3 and 6 nM, respectively) (FIG. 11E). The MCF7 line was used as a non IL-6, non IL-6R expressing cell line as published by others (Zhong et al., 2016).

FIG. 12 TfR1 dependent internalization of IL-6 by IL6R negative MCF7 cell line by the BsAb. MCF7 cells were plated on cover slips like CFPAC cells in FIG. 10. After 3 days, (FIG. 12A) IL-6 (200 ng/mL) alone (left panel) or combined with BsAb (30 μg/mL) (center panel) or combined to a mix of BsAb and an excess holo-Tf (10 μM) or (FIG. 12B) a mix IL6 (200 ng/mL) alone (left panel) or combined to BsAb (30 μg/mL) (right panel) were added for 1 hr. at 4° C. or 37° C. as indicated. Cells were then permeabilized, incubated with an anti-human-IgG1 conjugated with FITC and the mouse anti-IL6 BE8 followed by anti-mouse-IgG conjugated to PE. In FIG. 12A, fluorescence of FITC is shown (detection of the BsAb), in FIG. 12B, a merge of fluorescence due to FITC and PE is shown. The results in FIG. 12 A show that IL-6 does not internalize alone in MCF7 cells, that BsAb is internalized by MCF7 cells and that inernalization of BsAb is blocked by an excess of ligand holo-Tf. The results in FIG. 12B show that BsAb mediates IL-6 uptake in MCF-7 cell.

FIG. 13A IL-6 dependent XG6 myeloma cells were incubated in 96-U-bottom well plates in the presence of 4 pM IL6 (10 times EC₅₀) and increasing concentrations of either the non-neutralizing anti-IL6 VH4, or the anti-TfR1 F12 or of a combination of VH4 and F12, or of BsAb, “Bispecific” on the figure (70 pM to 70 nM) for 5 days in IMDM 10% FCS buffered at pH 7.2 to avoid pH drop due to metabolic activity. Cell viability was analyzed using the Cell titer Glow assay. (FIG. 13B-13C) same set up than in FIG. 13A with 4 pM IL6 and 3 concentrations (0.4, 0.1 and 0.04 nM) of various antibody treatments revealed by a viability assay (FIG. 13B) and phase microscope cliche (FIG. 13C). BsAb (0.4 nM) is more effective than the combination treatment of the parental antibodies VH4 and F12 at the same concentration (0.4 nM each) (13D) XG7 myeloma cell line was treated as indicated for 2 days. BsAb reduced STAT3 phosphorylation indicating reduced IL-6 signaling.

In [FIG. 14], XG6 (IL-6 dependent) and Raji (IL-6 independent) cell lines were treated for 3 days with the indicated concentrations of antibodies (FIG. 14A) neutralizing anti-IL6 mouse antibody BE8, (FIG. 14 B) BsAb (“Bispé” on the Figure) or parental monoclonal antibodies alone (“F12” or “VH4” on the Figure) (RPMI 10% FCS, HEPES 25 mM and IL6 at 4 pM) and their viability was assessed by a cell titer Glow assay. In FIG. 14 C, IC₅₀ are indicated in nM. The ratio IC₅₀BsAb/IC₅₀F12 in also indicated.

In [FIG. 15], XG7 and XG6 IL-6 dependent myeloma cell lines were treated for 3 days (RPMI 1% FCS, HEPES 25 mM and IL6 4 pM) with the indicated concentrations of antibodies BsAb or parental mAbs combination and their viability was assessed by a cell titer glow assay. Viability is expressed as % compared to non-treated cells (NT).

FIG. 16 schematizes scFv-Fc format of bispecific antibody.

FIG. 17 represents measurement of bispecific antibodies affinity for native TfR1 by FACS analysis.

FIG. 18 represents the inhibition of holo-TTf internalization at 37° C. by the three bispecific antibodies.

FIG. 19 represents the pH dependent binding of bispecific antibodies to human IL-6.

FIG. 20 represents the binding to recombinant mouse IL-6 or human IL-6 at pH 7.4 FIG. 21.

FIG. 21 represents the detection of the internalization of bispecific antibodies on the MCF7 breast cancer cell line by immunofluorescence (IL-6R negative) FIG. 22.

FIGS. 22 A to D represent TfR1 modulation by the bispecific antibodies formats by measurement of TfR1 protein level expression on XG-6 cells treated after 48 hours of treatment with bispecific formats or parental antibody H7-IgG1.

FIG. 22A histogram overlays of non-stained (light grey), or TfR1 stained (medium grey) non treated cells, and from left to right (dark grey) TfR1 staining of H7-IgG1, tetra-BsAb or scFv-Fc treated cells, respectively;

FIG. 22B represents the Mean Fluorescent Intensity (MFI) value in function of the treatment.

FIG. 22C represents a Western Blot for total TfR1 level quantification.

FIG. 22D represents total TfR1 quantification.

FIG. 23 A to D represent the characterization of Fab-scFv and scFv-Fc BsAbs sweeping activity in vivo

FIG. 23A represents the viability after 5 days of incubation with anti-TfR1 H7-IgG1.

FIG. 23B represents the average of tumor sizes in each group at different time (days).

FIG. 23C represents tumor size in each treatment group.

FIG. 23D represents the quantification of plasma human IL-6 at sacrifice

### DISCLOSURE OF THE INVENTION

The present invention concerns a bispecific antibody comprising:
A first antigen-binding domain which binds competitively to transferrin receptor 1 (TfR1);
A second antigen-binding domain which binds to a soluble antigen, wherein the soluble antigen dissociate from the second antigen-binding domain into endosome.

### Bispecific Antibody

The making of bispecific antibodies is known from the one skilled in the art and should be understood broadly as comprising around 100 different formats including small molecules composed solely of the antigen binding sites of two antibodies, molecules with IgG structure, and large complex molecules composed of different antigen-binding moieties, molecules with an IgG structure, and large complex molecules composed of different antigen-binding moieties often combined with dimerization modules.

Hence, as used herein, the term “bispecific antibody” should be understood as antibodies that recognize two different epitopes either on the same or on different antigens.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A. et al, EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M. et al, Science 229 (1985) 81-83); using leucine zippers to produce bi-specific antibodies (see, e.g., Kostelny, S. A. et al, J. Immunol. 148 (1992) 1547-1553; using “diabody” technology for making bispecific antibody fragments (see, e.g., HoUiger, P. et al, Proc. Natl. Acad. Sci. USA 90 (1993) 6444-6448); and using single-chain Fv (scFv) dimers (see, e.g. Gruber, M et al, J. Immunol. 152 (1994) 5368-5374); and preparing trispecific antibodies as described, e.g., in Tutt, A. et al, J. Immunol. 147 (1991) 60-69).

Brinkmann and al., in “The making of bispecific antibodies”, (MABS; 2017, VOL. 9, NO. 2, 182-212) focuses on the various formats and strategies available to generate recombinant bispecific antibodies. Recombinant bispecific antibodies can be classified according to format and composition. A main discrimination is the presence or absence of an Fc region. Bispecific antibodies with no Fc will lack Fc-mediated effector functions and could be classified as “Fc-less bispecific antibody formats”.

Bispecific antibodies that include an Fc region can be further divided into those that exhibit a structure resembling that of an IgG molecule and those that contain additional binding sites, i.e., those with an appended or modified Ig-like structure. The different bispecific antibodies will have either a symmetric or an asymmetric architecture.

For example, the majority of bispecific IgG molecules are asymmetric, while IgG fusion proteins often are symmetric in their molecular composition.

Hence, Brinkamann and al. described in the previous cited article (“the making of bispecific antibodies”) various formats of bispecific antibodies classified in three categories: i) Fc-less bispecific antibody formats; ii) Bispecific IgGs with asymmetric architecture; iii) Bispecific antibodies with a symmetric architecture.

Format and strategies to generate recombinant bispecific antibodies for each of these three categories are detailed in Brinkmann and al., which is incorporated herein by reference.

### Fc-Less Bispecific Antibody Formats

Fc-less bispecific antibody formats comprise:
Tandem single-chain variable fragments (scFv2, taFv) and triplebodies;
Bispecific single-domain antibodyfusion proteins;
Diabodies and diabody derivatives;
Fab fusion protein
Other Fc-less fusion proteins;
Additional antigen-binding sites grafted onto scFv.
   Bispecific IgGs with Asymmetric Architecture

Bispecific IgGs with asymmetric architecture comprise:
Asymmetric IgGs with heavy chain and light chains from two different antibodies;
Bispecific IgGs with an asymmetric Fc region;
Asymmetric Fc and CH3 fusion proteins.

Post-assembly approaches to purify bispecific antibodies, genetic engineering for solving the light chain problem in asymmetric antibodies and post-production assembly from half-antibodies for solving the light chain problem are also described and incorporated by reference.

Bispecific Antibodies with a Symmetric Architecture

Bispecific antibodies with a symmetric architecture comprise:
Appended IgGs: fusion of scFv;
Appended IgGs: fusion of domain antibodies and scaffold proteins;
Appended IgGs: fusion of Fab arms;
Appended IgGs: fusion of additional variable heavy and light chain domains;
Modified IgG molecules;
Symmetric Fc- and CH3-based bispecific antibodies;
Bispecific antibodies using immunoglobulin-derived homodimerization domains.

Hence, the one skilled in the art knows the best format for generating bispecific antibodies and knows how to generate by biochemical or genetic means bispecific antibodies.

Typically, the one skilled in the art can selected bispecific antibodies among:
Bispecific antibody conjugates such as IgG2, F(ab′)₂, CovX-Body;
Hybrid bispecific IgGs such as IgG, mouse/rat chimeric IgG, K/k-body common HC;
“variable domain only” bispecific antibody molecules such as tandem scFv (taFv), triplebodies, diabody (db), DsDb, db(kih), DART, scDb, dsFv-dsFv′, tandAbs, tripleheads, tandem dAb/VHH, triple dAb/VHH, tetravalent dAb/VHH;
Ch1/CL fusion proteins such as scFv₂-CH1/CL, VHH2-CH1/CL;
Fab fusion proteins such as Fab-scFv (bibody), Fab-scFv₂ (tribody), Fab-Fv, Fab-dsFv, Fab-VHH, orthogonal Fab-Fab;
Non-immunoglobulin fusion proteins such as scFv₂-albumin, scDb-albumin, taFv-albumin, taFv-toxin, miniantibody, DNL-Fab₃, DNL-Fab₂-scFv, DNL-Fab₂-IgG-cytokine₂, ImmTAC(TCR-scFv);
Fc-modified IgGs such as IgG(kih), IgG(kih) common LC, ZW1 IgG common LC, Biclonics common LC, CrossMab (IgG-kih), scFab-IgG(kih), orthogonal Fab IgG(kih), DuetMab, CH3 charge pairs+CH1/CL charge pairs, hinge/ch3 charge pairs, duobody, four-in-one-CrossMab (kih), LUZ-Y common LC, LUZ-Y scFab-IgG, LUZ-Y scFab-IgG, FcFc;
Appended & Fc-modified IgGs such as IgG(kih)-Fv, IgG(HA-TF-Fv), IgG(kih)-scFab, scFab-Fc(kih)-scFv2, scFab-Fc(kih)-scFv, half DVD-Ig, DVI-Ig (four-in-one), CrossMab-Fab,
Modified Fc and CH3 fusion proteins such as scFv-Fc(kih), scFv-Fc(CH3 charge pairs), ScFv-Fc(EW-RVT), scFv-Fc(HA-TF), scFv-Fc(SEEDbody), taFv-Fc(kih), scFv-Fc(kih)-Fv, Fab-Fc(kih)-scFv, Fab-scFv-Fc(kih), Fab-scFv-Fc(BEAT), Fab-scFv-Fc(SEEDbody), DART-Fc, scFv-C3(kih), TriFabs;
Appended IgGs—HC fusions such as IgG-HC-scFv, IgG-dAb, IgG-taFv, IgG-CrossFab, IgG-orthogonal Fab, IgG-(CaCO)Fab, scFv-HC-IgG, tandem Fab-IgG(orthogonal Fab);
Fab-IgG(CαCβFab), Fab-IgG(CR3), Fab-hinge-IgG(CR3);
Appended IgGs—LC fusions such as IgG-scFv(LC), scFv(LC)-IgG, dAb-IgG;
Appended IgGs—HC&LC fusions such as DVD-Ig, TVD-Ig, CODV-Ig, scFv4-IgG, zybody;
Fc fusions such as Di-diabody, scDb-Fc, taFv-Fc, scFv-Fc-scFv, HCAb-VHH, Fab-scFv-Fc, scFv₄-Ig, scFv₂-Fcab;
CH3 fusions such as di-diabody, scDb-CH3;
IgE/IgM CH2 fusions such as scFv-EHD2-scFv, scFv-MHD2-scFv;
F(ab′)₂ fusions such as F(ab′)₂-scFv₂;
CH1/CL fusion proteins such as scFv2-CH1-hinge/CL;
Modified IgGs such as DAF(two-in-one-IgG), DutaMab, mAb²;
Non-immunoglobulin fusions such as DNL-Fab₄-IgG.

In one embodiment, Fc-modified IgGs such as IgG(kih), IgG(kih) common LC, ZW1 IgG common LC, Biclonics common LC, CrossMab (IgG-kih), scFab-IgG(kih), orthogonal Fab IgG(kih), DuetMab, CH3 charge pairs+CH1/CL charge pairs, hinge/ch3 charge pairs, duobody, four-in-one-CrossMab (kih), LUZ-Y common LC, LUZ-Y scFab-IgG, LUZ-Y scFab-IgG, FcFc will be used.

In one embodiment, the bispecific antibody is a tetravalent IgG1-like chimeric bispecific antibody, a Fab-scFv, a Fab-scFv2, a scFv-Fc or a knob into hole antibody bispecific antibody, and preferably a knob into hole antibody or scFv bispecific antibody.

Tetravalent IgG1-like chimeric bispecific antibody format is described in Golay et al, (Journal of immunology; 2016). and scFv-Fab ou (scFv)2-Fab formats are described in Panke C et al. (Protein Engineering, Design & Selection vol. 26 no. 10 pp. 645-654, 2013).

### Internalization

The term “internalization”, “internalized” or “internalizing” when used in reference to a cell refers to the transport of a moiety (e.g. a bispecific antibody) from outside to inside a cell. The internalized moiety can be located in an intracellular compartment such as the endosome, a vacuole, a lysosome, the endoplasmic reticulum, the golgi apparatus or in the cytosol of the cell itself.

Hence, and according to the present invention, an internalizing receptor is a molecule present on the external cell surface that when specifically bound by an antigen-binding domain of a bispecific antibody according to the invention results in the internalization of complex formed by the bispecific antibody and the receptor into the cell.

Internalization of Transferrin receptor 1 (TfR1) in endosome is due to the binding of transferrin to TfR1. It also has been described that TfR1 can be internalized in the absence of ligand but at a lower rate (Kurten, R. C. (2003). Sorting motifs in receptor trafficking. Adv. Drug Deliv. Rev. 55, 1405-1419).

### Competitive Binding

According to the present invention, the first antigen-binding domain binds competitively to transferrin receptor 1 (TfR1). According to the present invention, the terms “competitive binding”, “binds competitively”, “competitive binding compound” or “competitive antigen-binding domain” relate to a compound that compete with the transferrin for binding to TfR1.

Typically, the competitive binding of an antigen-binding domain to TfR1 could be determined by the co-incubation of the ligand with the antigen binding domain. A binding can be considered as competitive if the co-incubation of the ligand with the antigen binding domain reduces the binding of the ligand to the receptor and vice versa.

Typically, the competitive binding of an antigen-binding domain can be evaluated by FACS analysis.

Hence, the first-antigen binding domain binds competitively with transferrin to bind to TfR1. The binding of the first antigen-binding domain of the bispecific antibody according to the invention to TfR1 induce internalization in the endosome of TfR1 and the bispecific antibody.

### Ligand-Like Binding

Furthermore, the first antigen-binding domain prevents the degradation of the receptor. Indeed, the binding of transferrin to TfR1 does not induce the degradation of TfR1, nor the binding of the first antigen-binding domain to TfR1.

### Recycling

Once Holo-Tf binds to TfR1, the complex enters the cell through clathrin-mediated endocytosis. Upon maturation and loss of the clathrin coat, the endosome pH will become acidified to a pH of about 5.5 At this pH, the binding of iron to Tf is weakened, leading to iron release from the Holo Transferrin. Most Transferrin receptor 1 returns to the cell surface (Ciechanover et al., 1983) where Apo-Tf is released since it has a low affinity for the receptor a the extra cellular pH.

### Soluble Antigen

In an embodiment, the soluble antigen is a pro-tumoral factor.

In an embodiment, pro-tumoral factors are those which act directly or indirectly on tumoral cells, and more particularly on growth, invasion metastasis, or angiogenesis.

For example, pro-tumoral soluble factors comprise cytokines, growth factors, matrix metalloproteinases (MMPs) family, urokinase-type plasminogen activator (uPA), soluble E-selectin.

Typically, cytokines can be interleukin such as IL-1, IL-6, IL-12, IL-15, IL-17 family, IFN-γ, M-csf, GM-CSF, Mif, Fas ligand, members of the TNF-α superfamily such as TNF-α, RANK-L, osteopontin (OPN).

IL-6 is well known in promoting tumor cell proliferation, survival, and angiogenesis. (Fisher et al., “The two faces of IL-6 in the tumor microenvironment”, Semin Immunol. 2014 February; 26(1):38-47).

TNF-α is also well known as a multifunctional cytokine playing a key role in apoptosis (via TNFR-1 on endothelial vessels) and cell survival (via TNFR-2 on immune cells, including regulatory T-cells and myeloid derived suppressive cells MDSC) (Shaikh, F et al. “TNF Receptor Type II as an Emerging Drug Target for the Treatment of Cancer, Autoimmune Diseases, and Graft-Versus-Host Disease: Current Perspectives and In Silico Search for Small Molecule Binders” Front Immunol. 2018; 9: 1382., Published online 2018 Jun. 18. doi: 10.3389/fimmu.2018.01382.

Rittling et al., in “Role of osteopontin in tumour progression” (British Journal of Cancer (2004) 90, 1877-1881) describe the importance of OPN in the process of tumorigenicity and metastasis.

Dranoff, in “Cytokines in cancer pathogenesis and cancer therapy” (Nature reviews, volume 4, January 2004) describe the role of IL-1, IL-6, IL-12, IL-15, Ifn-γ, M-csf, Gm-csf, Mif, Fas ligand, in tumor formation.

The IL-17 family comprises at least six members, IL-17A, IL-17B, IL-17C, IL-17D, IL-17E (also called IL-25), and IL-17F which have a promoting role in carcinogenesis, tumor metastasis and resistance to chemotherapy of diverse solid cancers (Fabre et al. (2016). “Targeting the Tumor Microenvironment: The Protumor Effects of IL-17 Related to Cancer Type”, International Journal of Molecular Sciences 17, 1433)

Renenma et al. (“RANK-RANKL signaling in cancer”, Bioscience reports (2016)) describe that RANKL is involved in all stages of tumorigenesis, including tumour hyperplasia, pre-neoplasia foci formation, cancer cell migration, neo-angiogenesis, immune cell chemo attraction and the establishment of an immunosuppressive environment and initiation of a pre-metastatic niche.

Typically, growth factors can be members of the vascular endothelial growth factor (VEGF) family such as VEGF, VEGFA, transforming growth factor such as TGF-β, Hepatocytes growth factor (HGF), basic fibroblast growth factor (bFGF), Epidermal Growth Factor Recceptor (EGFR) ligands family such as EGF, transforming growth factor α, amphiregulin, betacellulin, epiregulin and EGFR-like ligands family such as NRG1, NRG2, NRG3, NRG4.

Ferrara in “Role of vascular endothelial growth factor in regulation of physiological angiogenesis” (Am J Physiol Cell Physiol 280: C1358-C1366, 2001) describes the role of VEGF family in developmental and pathological angiogenesis, and emphasizes the 20 promising results of anti-VEGF antibody in cancer patients.

Furthermore, Esparis-Ogando et al. describe the importance of EGF ligands and EGF like ligands families in tumor generation and/or progression. «Targeting the EGF/HER Ligand-Receptor System in Cancer» Curr Pharm Des. 2016; 22(39):5887-5898.

TGF-β is a well-known tumor promoter and notably increases angiogenesis and growth of tumor cells (Yang et al., “TGF-β and immune cells: an important regulatory axis in the tumor microenvironment and progression”, Trends Immunol. 2010 June; 31(6):220-227).

Typically, matrix metalloproteinases can be MT1-MMP, MMP-1, MMP-2, MMP-3, MMP-8, MMP-9, MMP-10, MMP-11, MMP-13, MMP-14, MMP-19.

Gialeli et al. describe in “Roles of matrix metalloproteinases in cancer progression and their pharmacological targeting” (FEBS Journal 278 (2011) that MT1-MMP, MMP-1, MMP-2, MMP-3, MMP-8, MMP-9, MMP-10, MMP-11, MMP-13, MMP-14, MMP-19 are implied in cancer cell invasion, cancer cell proliferation and tumor angiogenesis and vasculogenesis.

Leonardi and al., in “tumor microenvironment in hepatocellular carcinoma” (international journal of oncology, 40:1733-1747, 2012) describe that soluble E-selectin promotes metastasis.

Urokinase-type plasminogen activator (uPA) plays a key role in tumor invasion and metastasis (Harbeck et al., “Urokinase-type plasminogen activator (uPA) and its inhibitor PAI-I: novel tumor-derived factors with a high prognostic and predictive impact in breast cancer”, Thrombosis and Hemostasis, April 2004).

In an embodiment pro-tumoral factors are those which act on tumor environment and provide factors necessary for cancer cell survival, dormancy (and therefore resistance to chemotherapy), proliferation or/and migration, and immune escape.

For example, pro-tumoral factors comprise cytokines, growth factors, cyclooxygenases, matrix metalloproteinases (MMPs) family, hepatitis B virus (HBV) X protein (HBx), Nonstructural proteins (nsPs) of Hepatitis C virus (HCV) (HCV-nsPs), prostaglandins, chemokines, Galectin-3 binding protein (Gal-3BP), Bcl-2-associated athanogene 3 (BAG3), PD-L1.

Typically, cytokines can be IL-8, IL-10, IL-23.

Waugh, D. J. J., and Wilson, C. (2008) “The interleukin-8 pathway in cancer”. Clin. Cancer Res. 14, 6735-6741, describe the secretion of IL-8 from cancer cells can enhance the proliferation and survival of cancer cells through autocrine signaling pathways.

IL-8, IL-10 and IL-23 are significantly associated with the direct number of circulating bone marrow (BM)-derived mesenchymal or very small embryonic/epiblast-like stem cells (Scs) in patient pancreatic cancer (“Selected cytokines in patients with pancreatic cancer” Blogowski et al.) 2014).

Typically matrix metalloproteinases (MMPs) family can be MMP9, MMP13, MMP14, MMP28, MMP8.

MMP9, MMP13, MMP14, MMP28, MMP8 have been described in Gialeli et al. (“Roles of matrix metalloproteinases in cancer progression and their pharmacological targeting” (FEBS Journal 278 (2011)) as acting on cell adhesion, migration, and epithelial to mesenchymal transition.

Typically, cyclooxygenases can be COX2 which as been described as an oncogene and a suppressor of tumor immunity by Liu et al. (“Cylclooxygenase-2 promotes tumor growth and suppresses tumor immunity”, Cancer Cell Int (2015) 15:106.

Hepatitis B virus (HBV) X protein (HBx) and Nonstructural proteins (nsPs) of Hepatitis C virus (HCV) (HCV-nsPs). HCV and HBV are involved in the genesis of hepatocarcinoma (HCC). Several proteins encoded by HCV et HBV are able to directly alter cytokine expression and directly modulate the tumor microenvironment and the immune response in the liver contributing to HCC development (Giulia et al., “The tumor microenvironment in hepatocellular carcinoma (review)”, international journal of oncology, 40:1733-1747, 2012).

Suppressor myeloid (MDSC) or T cells (Treg) attracting chemokines such as CXCL1, CXCL5, CCL2 and CCL12 who play a role in promoting tumor growth. Indeed, Giraldo et al. describe that the inflammatory milieu can promote tumor growth through the production of cytokines such as IL-6, IL-1 or TNF-α, in addition to angiogenic molecules such as VEGFA, transforming growth factor β (TGF-β), adenosine, prostanglandin E2 and suppressor myeloid or T cells attracting chemokines, including CXCL1, CXCL5, CCL2 and CCL12 (“The immune contexture of primary and metastatic human tumours”, Current opinion in Immunology 2014, 27:8-15)).

Programmed death-ligand 1 (PD-L1; also called B7-H1 or CD274), which is expressed on many cancer and immune cells, plays an important part in blocking the ‘cancer immunity cycle’ by binding programmed death-1 (PD-1) and B7.1 (CD80), both of which are negative regulators of T-lymphocyte activation. Binding of PD-L1 to its receptors suppresses T-cell migration, proliferation and secretion of cytotoxic mediators, and restricts tumour cell killing (Herbst et al. “Predictive correlates of response to the anti-PD-L1 antibody MPDL3280A in cancer patients” (Nature. 2014 Nov. 27; 515(7528): 563-567.)).

Silverman et al. (“A galectin-3-Dependent Pathway Upregulates interleukin-6 in the microenvironment of Human Neuroblastoma”, Cancer Res; 72(9); 228-32) show that he production of IL-6 in Bone Marrow Mesenchymal Stem cells (BMMSCs) is in part stimulated by galectin-3 binding protein (Gal-3BP) secreted by neuroblastoma cells.

Rosati et al. describe that pancreatic ductal adenocarcinoma (PDAC) cells secrete BAG3, which binds and activates macrophages, inducing their activation and the secretion of PDAC supporting factors (“BAG3 promotes pancreatic ductal adenocarcinoma growth by activating stromal macrophages”, Nature Communications, 6:8695).

In a preferred embodiment, the pro-tumoral factor is selected from the group consisting of IL-6, PDL-1, GM-CSF, Gal-3BP, BAG3, IL-17 family such as IL-17A to IL-17F, IL-10, EGF, NRG1, NRG2, NRG3, NRG4, HGF, RANK ligand.

In a preferred embodiment, the pro-tumoral factor is IL-6.

In another embodiment, the soluble antigen is a pro-inflammatory factor.

Typically pro-inflammatory factors comprise cytokines, C5, growth factors and IgE.

Typically, cytokines can be interleukin such as IL-6, IL-1, IL-10, IL-2, IL-5, IL-6 soluble receptor, IL-12, IL-15, IL-21, IL-17 family and IL-23GcSF, TNF-α, soluble TNF-α receptor, BAFF.

Pro-inflammatory factor can also be C5 and IgE.

These molecules have been indeed targeted in various inflammatory pathologies as listed in the table below:

TABLE 1

| Antibody or therapeutic | International | |
|---|---|---|
| molecule | denomination | Indication |
| Anti-TNF-α antibody | Infliximab | inflammatory bowel disease; |
| | | psoriasis; rheumatologic |
| | | inflammatory pathologies; |
| | | multiple sclerosis |
| Anti-TNF-α antibody | Certolizumab | rheumatologic inflammatory |
| | | pathologies, psoriasis |
| Anti-TNF-α antibody | Adalimunab | inflammatory bowel disease; |
| | | psoriasis; |
| Anti-TNF-α antibody | Golimumab | rheumatologic inflammatory |
| Anti-soluble TNF-α | Etanercep | pathologies |
| receptor antibody | | |
| Anti-IL-6R-antibody | Tocilizumab | rheumatologic inflammatory |
| | | pathologies; NMO |
| Anti-IL-6 antibody | Sirikumab | rheumatoid arthritis |
| Anti-IL-1β antibody | Canakinumab | ankylosing spondylitis |
| Anti-II-5 antibody | Mepolizumab | Asthma |
| Anti-II-5 antibody | Reslimumab | |
| Anti-IL-17A antibody | Secukinumab | Psoriasis |
| Anti-IL-17RAantibody | Brodalumab | |
| Anti-IL-17A antibody | Ixekizumab | |
| Anti-sub-unit p40 | Ustekinumab | |
| shared by IL-12 and | | |
| IL23 antibody | | |
| Anti-C5 antibody | Eculizumab | rheumatologic inflammatory |
| | | pathologies |
| Anti-BAFF antibody | Belimumab | Autoimmune pathologies |
| | | such as systemic lupus, |
| | | inflammatory pathologies |
| Anti-IgE antibody | Omalizumab | allergy |

IL-15 is a pro-inflammatory, innate response cytokine that mediates pleiotropic effector function in rheumatoid arthritis (RA) inflammatory synovitis. Baslund B. et al. demonstrate that, based on clinical data, IL-15 could represent a novel therapeutic target in rheumatoid arthritis (Targeting interleukin-15 in patients with rheumatoid arthritis: a proof-of-concept study” Arthritis Rheum. 2005 September, 52(9):2686-92).

IL-21 promotes a range of autoimmune diseases, including systemic lupus erythematosus, type 1 diabetes, multiple sclerosis, inflammatory bowel disease and psoriasis.

Typically growth factors can be TGFβ. Korn et al. (“IL-21 initiates an alternative pathway to induce pro-inflammatory TH17 cells”, Nature. 2007 Jul. 26; 448(7152): 484-487) describe that TGFβ notably induce the differentiation of T_{H}17 cells.

In a preferred embodiment, the pro-inflammatory factor is selected from the group consisting of IL-6, TNF-α, soluble TNF-α receptor, IL-1β, IL-5, IL-17A, IL-12, IL-23, C5, BAFF, IgE and TGFβ.

In a preferred embodiment, the pro-inflammatory factor is selected among IL-6, and TGFβ.

Preferably, the pro-inflammatory factor is IL-6.

### Antigen-Binding Domain

The term “antigen-binding domain” or “antigen-binding fragment” denotes a molecule other than an intact antibody that comprises a portion of an intact antibody that retain the ability to specifically binds to a given antigen (e.g., TfR1 or a soluble antigen) to which the intact antibody specifically binds. Examples of binding fragments encompassed include but are not limited to Fv fragment, Fab fragment, a monovalent fragment consisting of the VL, VH, CL and CH1 domains; a Fab′ fragment, a monovalent fragment consisting of the VL, VH, CL, CH1 domains and hinge region; a F(ab′)2 fragment, a bivalent fragment comprising two Fab′ fragments linked by a disulfide bridge at the hinge region; an Fd fragment consisting of VH domains of a single arm of an antibody; a single domain antibody (sdAb) fragment, diabodies, linear antibody, single-chain antibody molecules (e.g. scFv), single-chain Fab fragments (scFab), single heavy chain antibodies (VHH).

Three highly divergent stretches within the variable region of the heavy and light chains are referred to as “hypervariable regions” which are interposed between more conserved flanking stretches known as “framework region” or “FRs”. Thus the term “FR” refers to amino acid sequences which are naturally found between and adjacent to hypervariable region. In an antibody molecule, three hypervariable regions of a light chain and three hypervariable regions of a heavy chain are disposed relative to each other in three dimensional space to form an antigen binding surface. This surface mediates recognition and binding of the target antigen. The three hypervariable regions of each of the heavy and light chain are referred to as “complementary determining regions” or “CDRs”-In one embodiment, the bispecific antibody according to the invention has two antigen-binding domains a first-antigen binding domain that binds to TfR1 and a second antigen-binding domain that binds to a soluble antigen.

### First Antigen-Binding Domain: Antigen-Binding Domain to TfR1

Typically, the antigen-binding domain that competitively binds to transferrin receptor 1 (TfR1) is an antigen-binding domain of an anti-TfR1 antibody.

Advantageously, antigen-binding domain according to the present invention does not induce degradation of the TfR1.

In one embodiment the antigen-binding domain that specifically binds to transferrin receptor 1 is an antigen-binding domain of an anti-TfR1 antibody chosen among H7, F12, C32, F2, H9, G9

Sequence of the heavy chain of H7 antibody (SEQ ID NO:1):

QVQLQESGGGVVQPGRSLRLSCAASRFTFSSYAMHWVRQAPGKGLEW
VAVISYDGSNKYYADSVKGRFTISRDNSKNTLYLQMNSLRAEDTAVY
YCARDLSGYGDYPDYWGQGTLVTVSS

Sequence of the light chain of H7 antibody (SEQ ID NO:2):

SELTQDPAVSVALGQTVRITCQGDSLRSYYASWYQQKPGQAPVLVMY
GRNERPSGVPDRFSGSKSGTSASLAISGLQPEDEANYYCAGWDDSLT
GPVFGGGTKLTVLG

Typically, Heavy chain and light chain can be linked by a peptide linker of SEQ ID No 3 (scFv format):

GGGGSGGGGSGGGGS

In one embodiment, sequence of antigen-binding domain of H7-antibody is (SEQ ID NO:4):

QVQLQESGGGVVQPGRSLRLSCAASRFTFSSYAMHWVRQAPGKGLEW
VAVISYDGSNKYYADSVKGRFTISRDNSKNTLYLQMNSLRAEDTAVY
YCARDLSGYGDYPDYWGQGTLVTVSSGGGGSGGGGSGGGGSSELTQD
PAVSVALGQTVRITCQGDSLRSYYASWYQQKPGQAPVLVMYGRNERP
SGVPDRFSGSKSGTSASLAISGLQPEDEANYYCAGWDDSLTGPVFGG
GTKLTVLG

Sequence of heavy chain of F12-antibody (SEQ ID NO:5):

QVQLQESGGGLVQPGGSLRLSCAASGFSFNTYTMHWVRQAPGKGLEW
VADIAYDGSTKYYADSVKGRFTISRDNAKNSLYLQMNSLRAEDTAVY
YCARDAVAGEGYFDLWGRGTLVTVSS

Sequence of the light chain of F12 antibody (SEQ ID NO:6):

QSALTQDPAVSVALGQTVRITCQGDSLRSYYASWYQQLPGTAPKLLI
YRNNQRPSGVPDRFSGSKSGTSASLAISGLRSEDEADYYCAAWDDSL
SAWVFGGGTKLTVLGA

Typically, heavy chain and light chain can be linked by a peptide linker of SEQ ID No 3 (scFv format):

GGGGSGGGGSGGGGS

In one embodiment, sequence of antigen-binding domain of F12-antibody is (SEQ ID NO:7):

QVQLQESGGGLVQPGGSLRLSCAASGFSFNTYTMHWVRQAPGKGLEW
VADIAYDGSTKYYADSVKGRFTISRDNAKNSLYLQMNSLRAEDTAVY
YCARDAVAGEGYFDLWGRGTLVTVSSGGGGSGGGGSGGGGSQSALTQ
DPAVSVALGQTVRITCQGDSLRSYYASWYQQLPGTAPKLLIYRNNQR
PSGVPDRFSGSKSGTSASLAISGLRSEDEADYYCAAWDDSLSAWVFG
GGTKLTVLGA.

Sequence of the heavy chain of C32 antibody (SEQ ID NO: 8):

VQLQESGGGLVQPGGSLRLSCAASGFTFSSYAMSWVRQAPGKGLEWV
SAISGSGGSTYYADSVKGRFTISRDNSKNTLYLQMNSLRAEDTAVYY
CAKVSSSWSHFDYWGQGTLVTVSS

Sequence of the light chain of C32 antibody (SEQ ID NO: 9):

DVVMTQSPSTLSASVGDRVTITCRASQYISNWLAWYQQKPGKAPKLL
IYKASSLESGVPSRFSGSGSGTEFTLTISSLQPEDFATYYCQESYNT
PLFTFGPGTKLEIKR

Typically, heavy chain and light chain can be linked by a peptide linker of SEQ ID No 3 (scFv format):

GGGGSGGGGSGGGGS

Sequence of antigen-binding domain of C32-antibody (SEQ ID NO:10):

QVQLQESGGGLVQPGGSLRLSCAASGFTFSSYAMSWVRQAPGKGLEW
VSAISGSGGSTYYADSVKGRFTISRDNSKNTLYLQMNSLRAEDTAVY
YCAKVSSSWSHFDYWGQGTLVTVSSGGGGSGGGGSGGGGSDVVMTQS
PSTLSASVGDRVTITCRASQYISNWLAWYQQKPGKAPKLLIYKASSL
ESGVPSRFSGSGSGTEFTLTISSLQPEDFATYYCQESYNTPLFTFGP
GTKLEIKR

The antigen-binding domain of C32-antibody comprises, according to Kabat (a) a heavy chain CDR1 comprising the amino acid sequence of SEQ ID NO:11, (b) a heavy chain CDR2 comprising the amino acid sequence of SEQ ID NO:12, (c) a heavy chain CDR3 comprising the amino acid sequence of SEQ ID NO:13, (d) a light chain CDR1 comprising the amino acid sequence of SEQ ID NO:14; (e) a light chain CDR2 comprising the amino acid sequence of SEQ ID NO:15, (f) a light chain CDR3 comprising the amino acid sequence of SEQ ID NO:16

TABLE 2

| C32 | KABAT | SEQ ID NO: |
|---|---|---|
| CDR1-H | SYAMS | SEQ ID NO: 11 |
| CDR2-H | AISGSGGSTYYADSVK | SEQ ID NO: 12 |
| CDR3-H | VSSSWSHFDY | SEQ ID NO: 13 |
| CDR1-L | RASQYISNWLA | SEQ ID NO: 14 |
| CDR2-L | KASSLES | SEQ ID NO: 15 |
| CDR3-L | QESYNTPLFT | SEQ ID NO: 16 |

The antigen-binding domain of C32-antibody comprises, according to IMGT (a) a heavy chain CDR1 comprising the amino acid sequence of SEQ ID NO:17, (b) a heavy chain CDR2 comprising the amino acid sequence of SEQ ID NO:18, (c) a heavy chain CDR3 comprising the amino acid sequence of SEQ ID NO:19, (d) a light chain CDR1 comprising the amino acid sequence of SEQ ID NO:20; (e) a light chain CDR2 comprising the amino acid sequence of SEQ ID NO:21, (f) a light chain CDR3 comprising the amino acid sequence of SEQ ID NO:22.

TABLE 3

| C32 | IMGT | SEQ ID NO |
|---|---|---|
| CDR1-H | GFTFSSYA | SEQ ID NO: 17 |
| CDR2-H | ISGSGGST | SEQ ID NO: 18 |
| CDR3-H | AKVSSSWSHFDY | SEQ ID NO: 19 |
| CDR1-L | QYISNW | SEQ ID NO: 20 |
| CDR2-L | KAS | SEQ ID NO: 21 |
| CDR3-L | QESYNTPLFT | SEQ ID NO: 22 |

Sequence of the heavy chain of F2 antibody (SEQ ID NO: 23):

QVQLQQSGGGVVQPGGSLRLSCAASEFTFSASGMHWVRQAPGKGLEW
MAFIAYDGNQKFYADSVKGRFTISRDNSKNTLYLQMDSLRGEDTAVY
YCAKEMQREGYFDYWGQGTLVTVSS

Sequence of the light chain of F2 antibody (SEQ ID NO: 24):

NFMLTQDPAVSVALGQTVRITCQGDSLRSYYASWYQQKPGQAPVLVIYGK
NNRPSGIPDRFSGSKSGNSASLDISGLQSEDEADYYCATWDDNLSGPIFG
GGTKVTVLG

Typically, heavy chain and light chain can be linked by a peptide linker of SEQ ID No 3 (scFv format):

GGGGSGGGGSGGGGS

Sequence of antigen-binding domain of F2-antibody (SEQ ID NO:25):

QVQLQQSGGGVVQPGGSLRLSCAASEFTFSASGMHWVRQAPGKGLEWMAF
IAYDGNQKFYADSVKGRFTISRDNSKNTLYLQMDSLRGEDTAVYYCAKEM
QREGYFDYWGQGTLVTVSSGGGGSGGGGSGGGGSNFMLTQDPAVSVALGQ
TVRITCQGDSLRSYYASWYQQKPGQAPVLVIYGKNNRPSGIPDRFSGSKS
GNSASLDISGLQSEDEADYYCATWDDNLSGPIFGGGTKVTVLG

The antigen-binding domain of F2-antibody comprises, according to Kabat (a) a heavy chain CDR1 comprising the amino acid sequence of SEQ ID NO:26, (b) a heavy chain CDR2 comprising the amino acid sequence of SEQ ID NO:27, (c) a heavy chain CDR3 comprising the amino acid sequence of SEQ ID NO:28, (d) a light chain CDR1 comprising the amino acid sequence of SEQ ID NO:29; (e) a light chain CDR2 comprising the amino acid sequence of SEQ ID NO:30, (f) a light chain CDR3 comprising the amino acid sequence of SEQ ID NO:31.

TABLE 3

| F2 | KABAT | SEQ ID NO: |
|---|---|---|
| CDR1-H | ASGMH | SEQ ID NO: 26 |
| CDR2-H | FIAYDGNQKFYADSVKG | SEQ ID NO: 27 |
| CDR3-H | EMQREGYFDY | SEQ ID NO: 28 |
| CDR1-L | QGDSLRSYYAS | SEQ ID NO: 29 |
| CDR2-L | GKNNRPS | SEQ ID NO: 30 |
| CDR3-L | ATWDDNLSGPI | SEQ ID NO: 31 |

The antigen-binding domain of F2-antibody comprises, according to IMGT (a) a heavy chain CDR1 comprising the amino acid sequence of SEQ ID NO:32, (b) a heavy chain CDR2 comprising the amino acid sequence of SEQ ID NO:33, (c) a heavy chain CDR3 comprising the amino acid sequence of SEQ ID NO:34, (d) a light chain CDR1 comprising the amino acid sequence of SEQ ID NO:35; (e) a light chain CDR2 comprising the amino acid sequence of SEQ ID NO:36, (f) a light chain CDR3 comprising the amino acid sequence of SEQ ID NO:37.

TABLE 4

| F2 | IMGT | SEQ ID NO: |
|---|---|---|
| CDR1-H | EFTFSASG | SEQ ID NO: 32 |
| CDR2-H | IAYDGNQK | SEQ ID NO: 33 |
| CDR3-H | AKEMQREGYFDY | SEQ ID NO: 34 |
| CDR1-L | SLRSYY | SEQ ID NO: 35 |
| CDR2-L | GKN | SEQ ID NO: 36 |
| CDR3-L | ATWDDNLSGPI | SEQ ID NO: 37 |

Sequence of the heavy chain of H9 antibody (SEQ ID NO: 38):

QVQLAESGGGLVKPGGSLRLSCAASGFTFSDYYMSWIRQAPGKGLEWVSY
ISTSGSSIYYVDSVKGRFTISRDNAKNSLYLQMDSLRDDDTAVYYCARDL
HGDYAFDSWGQGTLVTVSS

Sequence of the light chain of H9 antibody (SEQ ID NO: 39):

SELTQDPAVSVALGQTVRITCQGDSLRSYYASWYQQKPGQAPVLVIYGKN
NRPSGIPDRFSGSKSGNSASLDISGLQSEDEADYYCATWDDNLSGPIFGG
GTKVTVLG

Typically, heavy chain and light chain can be linked by a peptide linker of SEQ ID No 3 (scFv format):

GGGGSGGGGSGGGGS

Sequence of antigen-binding domain of H9-antibody (SEQ ID NO:40):

QVQLAESGGGLVKPGGSLRLSCAASGFTFSDYYMSWIRQAPGKGLEWVSY
ISTSGSSIYYVDSVKGRFTISRDNAKNSLYLQMDSLRDDDTAVYYCARDL
HGDYAFDSWGQGTLVTVSSGGGGSGGGGSGGGGSSELTQDPAVSVALGQT
VRITCQGDSLRSYYASWYQQKPGQAPVLVIYGKNNRPSGIPDRFSGSKSG
NSASLDISGLQSEDEADYYCATWDDNLSGPIFGGGTKVTVLG

The antigen-binding domain of H9-antibody comprises, according to Kabat (a) a heavy chain CDR1 comprising the amino acid sequence of SEQ ID NO:41, (b) a heavy chain CDR2 comprising the amino acid sequence of SEQ ID NO:42, (c) a heavy chain CDR3 comprising the amino acid sequence of SEQ ID NO:43, (d) a light chain CDR1 comprising the amino acid sequence of SEQ ID NO:44; (e) a light chain CDR2 comprising the amino acid sequence of SEQ ID NO:45, (f) a light chain CDR3 comprising the amino acid sequence of SEQ ID NO:46

TABLE 5

| H9 | KABAT | SEQ ID NO: |
|---|---|---|
| CDR1-H | DYYMS | SEQ ID NO: 41 |
| CDR2-H | YISTSGSSIYYVDSVKG | SEQ ID NO: 42 |
| CDR3-H | DLHGDYAFDS | SEQ ID NO: 43 |
| CDR1-L | QGDSLRSYYAS | SEQ ID NO: 44 |
| CDR2-L | GKNNRPS | SEQ ID NO: 45 |
| CDR3-L | ATWDDNLSGPI | SEQ ID NO: 46 |

The antigen-binding domain of H9-antibody comprises, according to IMGT (a) a heavy chain CDR1 comprising the amino acid sequence of SEQ ID NO:47, (b) a heavy chain CDR2 comprising the amino acid sequence of SEQ ID NO:48, (c) a heavy chain CDR3 comprising the amino acid sequence of SEQ ID NO:49, (d) a light chain CDR1 comprising the amino acid sequence of SEQ ID NO:50; (e) a light chain CDR2 comprising the amino acid sequence of SEQ ID NO:51, (f) a light chain CDR3 comprising the amino acid sequence of SEQ ID NO:52.

TABLE 6

| H9 | IMGT | SEQ ID NO: |
|---|---|---|
| CDR1-H | GFTFSDYY | SEQ ID NO: 47 |
| CDR2-H | ISTSGSSI | SEQ ID NO: 48 |
| CDR3-H | ARDLHGDYAFDS | SEQ ID NO: 49 |
| CDR1-L | SLRSYY | SEQ ID NO: 50 |
| CDR2-L | GKN | SEQ ID NO: 51 |
| CDR3-L | ATWDDNLSGPI | SEQ ID NO: 52 |

Sequence of the heavy chain of G9 antibody (SEQ ID NO: 53):

QVQLVESGGGLVEPGGSLRLSCAASGFTFSNYAINWVRQAPGKGLEWVA
NIHHDGNGKYYVDSVEGRFTISRDNAKNSLYLQMDSLRAEDTAIYYCAR
DGYGGYLDLWGQGTLVTVSS

Sequence of the light chain of G9 antibody (SEQ ID NO: 54):

SELTQDPAVSVALGQTVRITCQGDSLRSYYASWYQQKPGQAPVLVIYGK
NNRPSGIPDRFSGSGSGNTASLTITGAQAEDEADYYCAAWDDSLSGPVF
GGGTKVTVLG

Typically, heavy chain and light chain can be linked by a peptide linker of SEQ ID No 3 (scFv format):

GGGGSGGGGSGGGGS

Sequence of antigen-binding domain of G9-antibody (SEQ ID NO:55):

QVQLVESGGGLVEPGGSLRLSCAASGFTFSNYAINWVRQAPGKGLEWVA
NIHHDGNGKYYVDSVEGRFTISRDNAKNSLYLQMDSLRAEDTAIYYCAR
DGYGGYLDLWGQGTLVTVSSGGGGSGGGGSGGGGSSELTQDPAVSVALG
QTVRITCQGDSLRSYYASWYQQKPGQAPVLVIYGKNNRPSGIPDRFSGS
GSGNTASLTITGAQAEDEADYYCAAWDDSLSGPVFGGGTKVTVLG

The antigen-binding domain of G9-antibody comprises, according to kabat (a) a heavy chain CDR1 comprising the amino acid sequence of SEQ ID NO:56, (b) a heavy chain CDR2 comprising the amino acid sequence of SEQ ID NO:57, (c) a heavy chain CDR3 comprising the amino acid sequence of SEQ ID NO:58, (d) a light chain CDR1 comprising the amino acid sequence of SEQ ID NO:59; (e) a light chain CDR2 comprising the amino acid sequence of SEQ ID NO:60, (f) a light chain CDR3 comprising the amino acid sequence of SEQ ID NO:61.

TABLE 7

| G9 | KABAT | SEQ ID NO: |
|---|---|---|
| CDR1-H | NYAIN | SEQ ID NO: 56 |
| CDR2-H | NIHHDGNGKYYVDSVEG | SEQ ID NO: 57 |
| CDR3-H | DGYGGYLDL | SEQ ID NO: 58 |
| CDR1-L | QGDSLRSYYAS | SEQ ID NO: 59 |
| CDR2-L | GKNNRPS | SEQ ID NO: 60 |
| CDR3-L | AAWDDSLSGPV | SEQ ID NO: 61 |

The antigen-binding domain of H9-antibody comprises, according to IMGT (a) a heavy chain CDR1 comprising the amino acid sequence of SEQ ID NO:62, (b) a heavy chain CDR2 comprising the amino acid sequence of SEQ ID NO:63, (c) a heavy chain CDR3 comprising the amino acid sequence of SEQ ID NO:64, (d) a light chain CDR1 comprising the amino acid sequence of SEQ ID NO:65; (e) a light chain CDR2 comprising the amino acid sequence of SEQ ID NO:66, (f) a light chain CDR3 comprising the amino acid sequence of SEQ ID NO:67.

TABLE 8

| G9 | IMGT | SEQ ID NO: |
|---|---|---|
| CDR1-H | GFTFSNYA | SEQ ID NO: 62 |
| CDR2-H | IHHDGNGK | SEQ ID NO: 63 |
| CDR3-H | ARDGYGGYLDL | SEQ ID NO: 64 |
| CDR1-L | SLRSYY | SEQ ID NO: 65 |
| CDR2-L | GKN | SEQ ID NO: 66 |
| CDR3-L | AAWDDSLSGPV | SEQ ID NO: 67 |

In one embodiment, the anti-TfR1 antibody format is chosen among a scFv antibody format, a scFv2-Fc antibody format.

In one embodiment, the anti-TfR1 antibody format is a scFv fused to a CH1-CH2-CH3 (kih) or fused to a Fab either to a CK, or to both CK and a CH1. It can also be a Fab in a IgG kih format or 2 Fabs in a tetravalent IgG.

Preferably, the antigen-binding domain that specifically binds to transferrin receptor 1 is an antigen-binding domain of H7 anti-TFR1 antibody, typically as described in SEQ ID NO: 4.

More preferably, the antigen-binding domain of H7-antibody comprises according to Kabat (a) a heavy chain CDR1 comprising the amino acid sequence of SEQ ID NO:68, (b) a heavy chain CDR2 comprising the amino acid sequence of SEQ ID NO:69, (c) a heavy chain CDR3 comprising the amino acid sequence of SEQ SEQ ID NO:70, (d) a light chain CDR1 comprising the amino acid sequence of SEQ ID NO:71; (e) a light chain CDR2 comprising the amino acid sequence of SEQ ID NO:72, (f) a light chain CDR3 comprising the amino acid sequence of SEQ ID NO:73.

According to Kabat numbering system, the CDRs of the heavy and light chain variable domain of H7 are as follows:

Heavy chain CDR1 comprising the amino acid sequence of SEQ ID NO:68:

SYAMH

Heavy chain CDR2 comprising the amino acid sequence of SEQ ID NO:69:

VISYDGSNKYYADSVKG

Heavy chain CDR3 comprising the amino acid sequence of SEQ ID NO:70:

DLSGYGDYPDY

Light chain CDR1 comprising the amino acid sequence of SEQ ID NO:71:

QGDSLRSYYAS

Light chain CDR2 comprising the amino acid sequence of SEQ ID NO:72:

GRNERPS

Light chain CDR3 comprising the amino acid sequence of SEQ ID NO:73:

AGWDDSLTGPV

According to IMGT numbering system, the CDRs of the heavy and light chain variable domain of H7 are as follows:

Heavy chain CDR1 comprising the amino acid sequence of SEQ ID NO:74:

RFTFSSYA

Heavy chain CDR2 comprising the amino acid sequence of SEQ ID NO:75:

ISYDGSN

Heavy chain CDR3 comprising the amino acid sequence of SEQ ID NO:76:

ARDLSGYGDYPDY

Light chain CDR1 comprising the amino acid sequence of SEQ ID NO:77:

SLRSYY

Light chain CDR2 comprising the amino acid sequence of SEQ ID NO:78:

GRN

Light chain CDR3 comprising the amino acid sequence of SEQ ID NO:79:

AGWDDSLTGPV

In another embodiment, the antigen-binding domain that specifically binds to transferrin receptor 1 is an antigen-binding domain of F12 antibody, typically as described in SEQ ID NO: 7).

More preferably, the antigen-binding domain of F12 antibody comprises, according to Kabat, (a) a heavy chain CDR1 comprising the amino acid sequence of SEQ ID NO:80, (b) a heavy chain CDR2 comprising the amino acid sequence of SEQ ID NO:81, (c) a heavy chain CDR3 comprising the amino acid sequence of SEQ ID NO:82, (d) a light chain CDR1 comprising the amino acid sequence of SEQ ID NO:83; (e) a light chain CDR2 comprising the amino acid sequence of SEQ ID NO:84, (f) a light chain CDR3 comprising the amino acid sequence of SEQ ID NO:85.

According to Kabat numbering system, the CDRs of the heavy and light chain variable domain of F12 are as follows:

Heavy chain CDR1 comprising the amino acid sequence of SEQ ID NO:80:

TYTMH

Heavy chain CDR2 comprising the amino acid sequence of SEQ ID NO:81:

DIAYDGSTKYYADSVKG

Heavy chain CDR3 comprising the amino acid sequence of SEQ ID NO:82:

DAVAGEGYFDL

Light chain CDR1 comprising the amino acid sequence of SEQ ID NO:83:

QGDSLRSYYAS

Light chain CDR2 comprising the amino acid sequence of SEQ ID NO:84:

RNNQRPS

Light chain CDR3 comprising the amino acid sequence of SEQ ID NO:85:

AAWDDSLSAWV

According to IMGT numbering system, the CDRs of the heavy and light chain variable domain of F12 are as follows:

Heavy chain CDR1 comprising the amino acid sequence of SEQ ID NO:86:

GFSFNTYT

Heavy chain CDR2 comprising the amino acid sequence of SEQ ID NO:87:

IAYDGSTK

Heavy chain CDR3 comprising the amino acid sequence of SEQ ID NO:88:

ARDAVAGEGYFDL

Light chain CDR1 comprising the amino acid sequence of SEQ ID NO:89:

SLRSYY

Light chain CDR2 comprising the amino acid sequence of SEQ ID NO:90:

RNN

Light chain CDR3 comprising the amino acid sequence of SEQ ID NO:91:

AAWDDSLSAWV

### Second Antigen-Binding Domain to a Soluble Pro-Tumoral Factor

In an embodiment, the antigen-binding domain that binds to a soluble pro-tumoral factor in a pH and/or calcium dependent way is an antigen-binding domain of the following antibodies: anti-IL-1 antibody, anti-IL-6 antibody, anti-IL12 antibody, anti-IL-15 antibody, anti-IL-17 A antibody, anti-IL-17 B antibody, anti-IL-17 C antibody, anti-IL-17 D antibody, anti-IL-17 E antibody, anti-IL-17 F antibody, anti-IFN-γ antibody, anti-M-CSF antibody, anti-Gm-CSF antibody, anti-MIF antibody, anti-Fas-ligand antibody, anti-TNFα antibody, anti-RANK-L antibody, anti-OPN antibody, anti-VEGF antibody, anti-VEGFA antibody, anti-TGF-β antibody, anti-HGF antibody, anti-bFGF antibody, anti-EGF antibody, anti-TGFα antibody, anti-amphiregulin antibody, anti-betacellulin antibody, anti-epiregulin antibody, anti-NRG1 antibody, anti-NRG2 antibody, anti-NRG3 antibody, anti-NRG4 antibody, anti-MT1-MMP antibody, anti-MMP-1 antibody, anti-MMP-2 antibody, anti-MMP-3 antibody, anti-MMP-8 antibody, anti-MMP-9 antibody, anti-MMP-10 antibody, anti-MMP-11 antibody, anti-MMP-13 antibody, anti-MMP-14 antibody, anti-MMP-19 antibody, anti-E-selectin antibody, anti-uPA antibody.

In an embodiment, the antigen-binding domain that binds to a soluble pro-tumoral factor in a pH and/or calcium dependent way is an antigen-binding domain of the following antibodies: anti-IL-8 antibody, anti-IL-10 antibody, anti-IL-23 antibody, anti-MMP9 antibody, anti-MMP13 antibody, anti-MMP14 antibody, anti-MMP28 antibody, anti-MMP8 antibody, anti-COX2 antibody, anti-HBx antibody, anti-HCV-nsPs antibody, anti-CXCL1 antibody, anti-CXCL5 antibody, anti-CCL2 antibody, anti-CCL12 antibody, anti-PDL1 antibody, anti-Gal3BP antibody, anti-BAG3 antibody.

In another embodiment, the antigen-binding domain that binds to a soluble pro-tumoral factor in a pH and/or calcium dependent way is an antigen-binding domain of the following antibodies: anti-IL-6 antibody, anti-PDL-1 antibody, anti-GM-CSF antibody, anti-GAL-3BP antibody, anti-BAG3 antibody, anti-IL-17A antibody, anti-IL-17B antibody, anti-IL-17C antibody, anti-IL-17D antibody, anti-IL-17E antibody, anti-IL-17F antibody, anti-EGF antibody, anti-NRG1 antibody, anti-NRG2 antibody, anti-NRG3 antibody, anti-NRG4 antibody, anti-HGF antibody, anti-RANK-L antibody.

In a preferred embodiment, the antigen-binding domain is an antigen-binding domain of anti-IL-6 antibody.

For example, the antigen-binding domain is an antigen-binding domain of the monospecific VH4 anti-IL-6. The monospecific VH4 anti-IL-6 has been described in Devanaboyina et al. (“The effect of pH dependence of antibody-antigen interactions on subcellular trafficking dynamics”, mAbs 5:6, 851-859; November/December 2013).

Light chain of VH4 anti-IL-6 (SEQ ID NO:92):

DIQMTQSPSTLSASIGDRVTITCRASEGIYHWLAWYQQKPGKAPKLLIY
KASSLASGAPSRFSGSGSGTDFTLTISSLQPDDFATYYCQQYSNYPLTF
GGGTKLEIK

Heavy chain of VH4 anti-IL-6 (of SEQ ID NO:93):

EVQLVQSGGGVVQPGESLRLSCAASGFTFSSYSMNWVRQAPGKGLEWVS
VIYSGGSTYYADSVKGRFTISRDNSKNTLYLQMNSLRAEDTAVYYCARE
VYHSSGYDDAFDIWGRGTMVTVSS

Preferably, the antigen-binding domain of the monospecific VH4 anti-IL-6 comprises according to Kabat (a) a heavy chain CDR1 comprising the amino acid sequence of SEQ ID NO:94, (b) a heavy chain CDR2 comprising the amino acid sequence of SEQ ID NO:95, (c) a heavy chain CDR3 comprising the amino acid sequence of SEQ ID NO:96, (d) a light chain CDR1 comprising the amino acid sequence of SEQ ID NO:97; (e) a light chain CDR2 comprising the amino acid sequence of SEQ ID NO:98, (f) a light chain CDR3 comprising the amino acid sequence of SEQ ID NO:99.

TABLE 9

| VH4 | KABAT | SEQ ID NO: |
|---|---|---|
| CDR1-H | SYSMN | SEQ ID NO: 94 |
| CDR2-H | VIYSGGSTYYADSVKG | SEQ ID NO: 95 |
| CDR3-H | YHSSGYDDAFDI | SEQ ID NO: 96 |
| CDR1-L | RASEGIYHWLA | SEQ ID NO: 97 |
| CDR2-L | KASSLAS | SEQ ID NO: 98 |
| CDR3-L | QQYSNYPLT | SEQ ID NO: 99 |

Preferably, the antigen-binding domain of the monospecific VH4 anti-IL-6 comprises according to IMGT (a) a heavy chain CDR1 comprising the amino acid sequence of SEQ ID NO:100, (b) a heavy chain CDR2 comprising the amino acid sequence of SEQ ID NO:101, (c) a heavy chain CDR3 comprising the amino acid sequence of SEQ ID NO:102, (d) a light chain CDR1 comprising the amino acid sequence of SEQ ID NO:103; (e) a light chain CDR2 comprising the amino acid sequence of SEQ ID NO:104, (f) a light chain CDR3 comprising the amino acid sequence of SEQ ID NO:105.

TABLE 10

| VH4 | IMGT | SEQ ID NO: |
|---|---|---|
| CDR1-H | GFTFSSYS | SEQ ID NO: 100 |
| CDR2-H | IYSGGST | SEQ ID NO: 101 |
| CDR3-H | EVYHSSGYDDAFDI | SEQ ID NO: 102 |
| CDR1-L | EGIYHW | SEQ ID NO: 103 |
| CDR2-L | KAS | SEQ ID NO: 104 |
| CDR3-L | QQYSNYPLT | SEQ ID NO: 105 |

### Second Antigen-Binding Domain to a Pro-Inflammatory Factor

In an embodiment, the antigen-binding domain that binds to a soluble pro-inflammatory factor in a pH and/or calcium dependent way is an antigen-binding domain of the following antibodies: anti-IL-6 antibody, anti-IL-1 antibody, anti-IL-1 antibody, anti-IL-2 antibody, anti-IL-5 antibody, anti-IL-6 soluble receptor antibody, anti-IL-12 antibody, anti-IL-15 antibody, anti-IL-21 antibody, anti-IL-17A antibody, anti-IL-17B antibody, anti-IL-17C antibody, anti-IL-17D antibody, anti-IL-17E antibody, anti-IL-17F antibody, anti-IL-23 antibody, anti-G-CSF antibody, anti-TNF-α antibody, anti-soluble TNF-α receptor 10 antibody, anti-C5 antibody, anti-BAFF antibody, anti-IgE antibody.

In another embodiment, antigen-binding domain can be derived from antigen-binding domain of the following approved antibody. Typically, if antigen-binding domain are not pH binding dependent, variable region engineering will be realized in order to enable the antibody to bind to an antigen in plasma and dissociate from the antigen in endosome

TABLE 11

| Antibody or therapeutic molecule | International denomination |
|---|---|
| Anti-TNF-α antibody | Infliximab |
| Anti-TNF-α antibody | Certolizumab |
| Anti-TNF-α antibody | Adalimunab |
| Anti-TNF-α antibody | Golimumab |
| Anti-soluble TNF-α receptor | Etanercep |
| Anti-IL-6R-antibody | Tocilizumab |
| Anti-IL-6 antibody | Sirikumab |
| Anti-IL-1β antibody | Canakinumab |
| Anti-Il-5 antibody | Mepolizumab |
| Anti-Il-5 antibody | Reslimumab |
| Anti-IL-17A antibody | Secukinumab |
| Anti-IL-17RA antibody | Brodalumab |
| Anti-IL-17A antibody | Ixekizumab |
| Anti-sub-unit p40 shared by | Ustekinumab |
| IL-12 and IL23 | |
| Anti-C5 antibody | Eculizimumab |
| Anti-BAFF antibody | Belimumab |
| Anti-IgE antibody | Omalizumab |

In a preferred embodiment, the antigen-binding domain that binds to a soluble pro-inflammatory factor in a pH and/or calcium dependent way is an antigen-binding domain of the following antibodies: anti-IL-6 antibody, anti-TNFα antibody, anti-soluble TNFα receptor antibody, anti-IL-5 antibody, anti-IL-17A antibody, anti-IL-12 antibody, anti-IL10 antibody, anti-IL23 antibody, anti-C5 antibody, anti-BAFF antibody, anti-IgE antibody, anti-TGFβ antibody.

Preferably, the antigen-binding domain is an antigen-binding domain of anti-IL-6 antibody or anti-IL-10 antibody or anti TGFβ antibody.

In one embodiment the antigen-binding domain that specifically binds to IL-6 is an antigen-binding domain of an anti-IL-6 antibody.

For example, the antigen-binding domain that specifically binds to IL-6 is an antigen-binding domain of the monospecific VH4 anti-IL-6 of as described above

Hence, in one embodiment of the present invention, the bispecific antibody is an anti-TfR1-anti-IL6 with pH dependent binding.

In one embodiment, the anti-TfR1-anti-IL6 is in the tetravalent antibody format published by Golay et al, JI, 2016. The first antigen-binding domain is an antigen-binding domain of anti-TfR1 F12, and the second antigen-binding domain is the antigen-binding domain of the anti-IL-6 VH4. In another format, the anti-TfR1 is a scFv fused to the Ck and the CH1 domain of the VH4 anti-IL-6 Fab domain.

pH-Dependent Antigen Binding

As described in Igawa et al., The environment of an endosome is strictly controlled by various factors, including proton pumps (such as H⁺-ATPase and Ca²⁺-ATPase), proteases (such as cathepsin), and membrane proteins (such as toll-like receptors and Rab proteins). The acidic condition in an endosome plays an important role in trafficking proteins in the pathway to lysosomal degradation. Endosome maintains its acidic pH (approximately pH 5-6) by H⁺-ATPase, and several membrane receptors utilize its acidic environment to dissociate from or associate to their ligands. The binding of an antibody to an antigen generally has similar affinity at both neutral (pH 7.4) and acidic pH (pH 5-6).

Hence, the term “pH-dependent antigen binding” as used herein covers any antigen-binding domain able to bind to an antigen in plasma and dissociate from the antigen in endosome.

In an embodiment of the invention, the second antigen-binding domain is an antigen-binding domain whose antigen-binding activity is higher in a neutral pH range than under an acidic range.

Typically, the antigen-binding domain is able to bind to an antigen in plasma at neutral pH (pH 7.4) and to dissociate from the antigen at acidic pH, typically at pH comprises between 5 and 6.

Variable region engineering can be used to modulate the interaction between an antigen and an antibody. The purpose of variable region engineering is to enable an antibody to bind to an antigen in plasma and dissociate from the antigen in endosome.

The one skilled in the art knows the technologies for generating an antibody that dissociates the antigen within the endosome. This technology, as describes in Igawa et al., “Sweeping antibody as novel therapeutic modality”, 2016 John Wiley & Sons A/S. Published by John Wiley & Sons Ltd, Immunological Reviews 270/2016 can be classified in two approaches:
A mutational approach that generates a pH dependent antibody from a non-pH dependent antibody that has been identified by conventional approaches;
De novo generation of an antibody with pH dependent or calcium-dependent binding to the antigen, using the conventional approach on a large screening scale to identify the very rare antibody that has such properties.
   Histidine Mutagenesis for Generating an Antibody with pH-Dependent Binding

A non-pH-dependent antibody with high affinity against the target can be obtained using an established method and can then be engineered into a pH-dependent antibody by introducing histidine residues into the CDR or FR of the parent antibody. Histidine has pKa of around 6 (depending on the surrounding environment) and is utilized in naturally occurring pH-dependent protein-protein interactions, such as the interaction between Fc and FcRn. Because of the pKa of the imidazole group, histidine residues are protonated at endosomal acidic pH, a change that destabilizes the antibody-antigen interaction in two ways. When the introduced histidine residues are directly involved in interacting with the antigen, histidine protonation results in destabilizing the antibody-antigen interaction directly, and when the histidine residues are involved in maintaining the conformation of the CDR, histidine protonation results in conformational change of the CDR, thereby destabilizing the antibody-antigen interaction indirectly.

Typically, an histidine-engineered pH-dependent antibody was derived from tocilizumab, a humanized anti-IL-6R antibody. Tocilizumab was engineered into a pH-dependent anti-IL-6 receptor antibody by introducing four histidine residues, two in the heavy chain (positions 27 and 31 in Kabat numbering) and two in the light chain (positions 32 and 53), Igawa T, et al. Antibody recycling by engineered pH-dependent antigen binding improves the duration of antigen neutralization. Nat Biotechnol 2010; 28:1203-1207. A crystal structure of tocilizumab Fab fragment in complex with IL-6R suggested that histidine residues at position 31 in the heavy chain and position 32 in the light chain were directly involved in the antibody-antigen interaction, while histidine residue at position 27 supports the conformation of heavy chain CDR1. When histidine residues are protonated in acidic endosome, the antibody-antigen interaction is destabilized both by the electrostatic repulsion between the arginine residue in the IL-6 receptor and the protonated histidine residue and by the conformational change in the heavy chain CDR1.

Devanaboyina S C, et al., The effect of pH dependence of antibody-antigen interactions on subcellular trafficking dynamics. mAbs 2013; 5:851-859. reported generating a pH-dependent binding antibody against IL-6 in the same way.

The histidine-based engineering is a general method for generating antibodies with pH-dependent binding. Hence the one skilled in the art knows the methods and how introducing histidine(s) in the appropriate position for providing pH dependency.

Using a Display Library to Generate an Antibody with pH-Dependent Antigen Binding

Bonvin et al. (“De novo isolation of antibodies with pH-dependent binding properties”. mAbs2015:7:294-302) described designing an antibody library consisting of heavy chain CDR3 enriched with histidine residues (i.e. containing 1 to 4 histidine residues in the heavy chain CDR3) and then isolating antibodies with pH-dependent binding to human CXCL10.

Murtaugh et al. (“A combinatorial histidine scanning library approach to engineer highly pH-dependent protein switches”, Protein Sci 2011; 20:1619-1631) described using a combinatorial histidine scanning library to engineer a conventional antibody into a pH-dependent binding antibody. They used a VHH antibody against RNase A as a model antibody, and a combinatorial library was constructed that consisted of different combinations of histidine and wildtype residues in the antibody-antigen interface, from which it was possible to isolate a pH-dependent VHH antibody against RNase A using the phage display.

Schroter et al. (“A generic approaches to engineer antibody pH-switches using combinatorial histidine scanning libraries and yeast display” mAbs 2015; 7:138-151) described using a combinatorial histidine scanning library to engineer adalimumab, an anti-TNF antibody, into a pH-dependent anti-TNF antibody. A combinatorial library consisting of combinations of histidine and wildtype residues in the CDR of adalimumab was displayed on yeast. After three rounds of selection (consisting of three steps of FACS sorting in each round: binding to TNF at pH 7.4, release of TNF at pH 6.0, and rebinding of labeled TNF at pH 7.4), a pH-dependent anti-TNF antibody could be isolated.

In one embodiment of the present invention, the bispecific antibody comprises an histidine residue at an amino acid residue in at least one of the CDRX of the antigen-binding domain, whereby the CDRX is determined according to kabat.

Generally, the CDRX of the antigen-binding domain is CDR1 or CDR3.

### Calcium-Dependent Antigen Binding

As also described by Igawa et al., in “Sweeping antibody as novel therapeutic modality”, 2016 John Wiley & Sons A/S. Published by John Wiley & Sons Ltd, Immunological Reviews 270/2016, calcium ion concentration between plasma and endosomes is also known to be different. Calcium ion concentration is reported to be 1.2-2 mM in plasma, which drops to 3-30 μM in endosomes after endocytosis.

Hence, “Calcium-dependent antigen binding” as used herein means that the binding activity to the soluble antigen changes depending on an ion concentration condition. Hence, “calcium-dependent antigen binding” means any antigen-binding domain which binds to the antigen at the calcium ion concentration in plasma and dissociates the antigen at the calcium ion concentration.

Typically, the antigen-binding domain is able to bind to an antigen in plasma at a calcium concentration comprises between 1.2 and 2 mM in the plasma and to dissociate from the antigen in the endosome, when the calcium concentration is comprised between 3-30 μM.

The one skilled in the art knows how to enable an antibody to bind to an antigen in plasma and dissociate from the antigen in endosome. Typically, Hironiwa N, et al., in “Calcium-dependent antigen binding as a novel modality for antibody recycling by endosomal antigen dissociation”. mAbs 2016; 8:65-73, reported a novel calcium-dependentanti-IL-6 receptor antibody identified from a naïve human antibody phage library. In a method similar to that used to identify a pH-dependent antibody from a phage library, a human naïve library was panned for IL-6 receptors in the presence of 2 mM calcium ion and the phage was washed with a buffer containing calcium ion. Subsequently, populations that were dissociated in the absence of calcium ion or in the presence of EDTA were recovered for the next round. After multiple rounds of selection, a calcium-dependent anti-IL-6 receptor antibody was isolated. This antibody was shown to dissociate an IL-6 receptor both in vitro and in vivo.

In one embodiment, the bispecific antibody according to the present invention is for use in eliminating a soluble antigen from the circulation.

In another embodiment, the bispecific antibody according to the present invention is for use in eliminating a soluble antigen from a zone of the body where TfR1 is overexpressed.

Typically, TfR1 is overexpresses in the tumor or in the inflamed zone where immune cells expressing TfR1 are numerous.

As the bispecific antibody according to the present invention enable to bind a soluble antigen in the plasma or in the extracellular medium and dissociate from the antigen in endosome, the bispecific antibody enhance the elimination of the soluble antigen from circulation or in a targeted fashion.

### Pharmaceutical Composition

The present invention also concerns a pharmaceutical composition comprising a bispecific antibody according to the invention and a pharmaceutically acceptable carrier.

A used herein, “pharmaceutically acceptable” refers to those compounds, materials, excipients, compositions or dosage forms which are, within the scope of sound medical judgment suitable for use in contact with the tissues of subjects, without excessive toxicity, irritation, allergic response or other problem complications commensurate with a reasonable benefit/risk ratio. Pharmaceutically acceptable carriers include but are not limited to solvents, dispersion media, coatings, stabilizing agents, diluents, preservatives, antibacterial and antifungal agents, isotonic agents, adsorption delaying agents, and the like.

Diluents can include water, saline, dextrose, ethanol, glycerol and the like. Isotonic agents can include sodium chloride, dextrose, mannitol, sorbitol and lactose, among others known to those skilled in the art. Stabilizers include albumin. Preservatives include merthiolate. A person skilled in the art will be aware of suitable carriers. Suitable formulations for administration by any desired route may be prepared by standard methods, for example by reference to well-known text such as Remington; The Science and Practice of Pharmacy.

The present invention also concerns a bispecific antibody according to the invention for use as a medicament.

One aspect reported herein is the use of a bispecific antibody as reported herein in the manufacture of a medicament.

The present invention also concerns a method for the treatment of a disease in a subject in need thereof comprising administering to said subject an effective amount of a bispecific antibody as described above.

### Treatment of Cancers

As tumors cells overexpress TfR1 and have increased iron needs, the present invention also concerns the use of bispecific antibody according to the present invention for treating cancers.

In one embodiment, the medicament is for the treatment of cancer.

In another embodiment, the present invention also concerns a method for the treatment of a cancer in a subject in need thereof comprising administering to said subject an effective amount of a bispecific antibody as described above.

All the embodiment disclosed above are encompassed in these aspects.

By an “effective amount” of bispecific antibody is meant a sufficient amount to treat cancers, at a reasonable benefit/risk ratio applicable to any medical treatment. It will be understood, however, that the total daily usage of the bispecific antibody will be decided by the attending physician within the scope of sound medical judgment. The specific therapeutically effective dose level for any particular subject in need thereof will depend upon a variety of factors including the stage cancer and the activity of the specific bispecific antibody employed, the age, body weight, general health, sex and diet of the subject, the time of administration, route of administration, the duration of the treatment; drugs used in combination or coincidental with the and like factors well known in the medical arts. For example, it is well known within the skill of the art to start doses of the compound at levels lower than those required to achieve the desired therapeutic effect and to gradually increase the dosage until the desired effect is achieved.

The term “treatment” or “method of treating” or its equivalent is not intended as an absolute term and, when applied to, for example, cancer refers to a procedure or course of action that is designed to reduce or eliminate or to alleviate one or more symptoms of cancer.

Often, a “treatment” or a “method of treating” cancer will be performed even with a low likelihood of success but is nevertheless deemed to induce an overall beneficial effect.

Treatment of cancer refers, for example, to delay of onset, reduced frequency of one or more symptoms, or reduced severity of one or more symptoms associated with the disorder.

In some circumstances, the frequency and severity of one or more symptoms is reduced to non-pathological levels.

More particularly, the term of “treatment” or a “method of treating” of cancer refers to an improvement of clinical behavioral or biological criteria in the subject.

As described in Shen et al., (“Transferrin receptor 1 in cancer: a new sight for cancer therapy”, Am J Cancer Res. 2018; 8(6): 916-931), iron uptake by transferrin receptor is the most important way for cancer cells to absorb iron, thus accumulating evidence has proven that TfR1 participated in tumor onset and progression, and its expression was dysregulated significantly in many cancers. Furthermore and more importantly, TfR1 has been verified to be abnormally expressed in various cancers.

Hence, the bispecific antibody according to the present invention can advantageously target TfR1 on many kinds of cancers.

In one embodiment, the cancer is selected from solid cancer such as pancreatic cancer, neuroblastoma, leukemia, lymphoma, breast cancer, cancer related cachexia, gastrointestinal cancer such as colorectal cancer, cholangiocarcinoma, carcinoma of the oral cavity, gastric cancer, Lung cancer such as small cell lung cancer, lung adenocarcinoma, Melanoma, ovarian cancer, prostate cancer, renal cancer, hepatocarcinoma; or multiple myeloma

### Pancreatic Cancer

Bharadwaj et al., in “Elevated Interleukin-6 and G-CSF in human pancreatic cancer cell conditioned medium suppress dendritic cell differentiation and activation” [cancer Res 2007; 67(11):5479-88] found that conditioned media from three different pancreatic cancer cell lines inhibit DC differentiation. IL-6 and G-CSF in the conditioned medium cooperate to exert the inhibitory effect on DC differentiation, maturation, and Ag presentation functions. The abberant activation of STAT3 is found to be responsible for the inhibitory effect on DC differenciation. Taken together, authors consider that their data suggest important immunomodulatory targets in pancreatic cancer, which may help to design immunotherapeutic regimens for pancreatic cancer patients.

Mace T A et al., in “IL-6 and PD-L1 antibody blockade combination therapy reduces tumour progression in murine models of pancreatic cancer” (Gut 2018; 67:320-332) demonstrate that combined blockade of IL-6 and PD-L1 elicits efficacy and extends survival therapy in highly aggressive PDAC (pancreatic ductal adenocarcinoma) models.

Wu et al., in “Targeting IL-17B-IL-17RB signaling with an anti-IL-17RB antibody blocks pancreatic cancer metastasis by silencing multiple chemokines” (JEM Vol. 212, No 3) describes that IL-17B-IL-17RB define a novel regulatory pathway in pancreatic cancer cells.

Rosati et al., in “BAG3 promotes pancreatic ductal adenocarcinoma growth by activating stromal macrophages” (DOI: 10.138/ncomms9695) describes that anti-BAG3 antibody reduce tumor growth and metastatic spreading.

Okada et al., 1998, describes increased serum levels of IL-6 detected in 54.5% of pancreatic cancer patients; significantly among the patients with weight loss

### Neuroblastoma

Silverman et al. (“A galectin-3-Dependent Pathway Upregulates interleukin-6 in the microenvironment of Human Neuroblastoma”, Cancer Res; 72(9); 228-32) show that he production of IL-6 in BMMSCs is in part stimulated by galectin-3 binding protein (Gal-3BP) secreted by neuroblastoma cells. They identified a distal region of the IL-6 promoter that contains 3 CCATT/enhancer binding protein (C/EBP) binding domains involved in the transcriptional upregulation of IL-6 by Gal-3BP. Gal-3BP interacted with Galectin-3 (Gal3) present in BMMSCs, and a Gal-3BP/Gal-3/Ras/MEK/ERK signaling pathway was responsible for the transcriptional upregulation of IL-6 in BMMSCs in which Gal-3 has a necessary function. In support of the role of this pathway in human neuroblastoma tumors, Gal-3BP was found to be present in tumor cells and in the adjacent extracellular matrix of 96% of 78 primary neuroblastoma tumor samples examined by immunohistochemistry. Considering the protumorigenic function of IL-6 in cancer, this tumor cell-stromal cell interactive pathway could be a target for anticancer therapy.

### Leukemia

Robak et al., 1999, describe that serum levels of IL-6 in chronic lymphocytic leukemia patients were not significantly different from that of control subjects, IL-6 levels in patients treated with cladribine significantly lower, especially in patients who achieved remission.

### Lymphoma

Fayad et al., 1998, describe elevated serum IL-6 levels seen in 25% of indolent non-Hodgkin's lymphomas, was predictive of poor outcome.

Seymour et al., 1997, also describe that IL-6 serum levels were frequently elevated in patients with Hodgkin's disease and that they normalize with remission.

Preti et al., 1997, describe elevated serum IL-6 levels in patients with diffuse large cell lymphoma.

### Breast Cancer

Benoy et al., 2002 describe that median serum IL-6 level were about 10 times higher in patients with metastatic disease than in those with localized disease. Zhang and Adachi, 1999 observe significantly higher serum IL-6 levels in patients with more than 1 metastatic site.

Yokoe et al., 1997 observe IL-6 and IL-8 levels significantly higher in patients with progressive disease.

### Cancer Related Cachexia

Yamashita and Ogawa, 2000 observe a decrease in IL-6 serum levels associated with subjective improvement following therapy.

Mantovani et al., 1998 observe high serum levels of IL-1, IL-6, and TNFα in advanced stage cancer patients, particularly those with cachexia.

### Gastrointestinal Cancer

De Vita et al., 2001 indicate that serum IL-6 levels were elevated in advanced gastrointestinal cancer patients and correlated with overall survival.

Kinoshita et al., 1999 describe that serum IL-6 levels are indicative of tumor proliferative activity in colorectal cancer patients.

Goydos et al., 1998 observe high serum levels of IL-6 mark patients with cholangiocarcinoma and correlate with tumor burden.

Jablonska et al., 1997 demonstrate that serum levels of IL-1, IL-6, and TNFα elevated in patients with squamous cell carcinoma of the oral cavity.

Wu et al., 1996, describe that mean serum levels of IL-6 were significantly higher in patients with gastric cancer

### Lung Cancer

Alexandrakis et al., 2000 describe that IL-6, IL-8, and TNFα were found in higher concentrations in malignant pleural effusion than in serum

Dowlati et al., 1999, indicate that serum IL-6 levels were higher in patients with extensive small cell lung cancer than in patients with limited-stage disease

Martin et al., 1999 demonstrate that increased IL-6 level was related to extensive disease, impaired performance status, and enhanced acute-phase response.

De Vita et al., 1998 indicate that mean IL-6 concentrations were significantly higher in non-small cell lung cancer patients than in control subjects.

Nakano et al., 1998 describe that serum concentration of IL-6 was significantly higher in mesothelioma than in lung adenocarcinoma.

### Melanoma

Moretti et al., 2001 indicate significantly higher serum IL-6 and IL-12 levels were observed in patients with localized and metastatic melanoma.

Mouawad et al., 1996 describe that baseline serum IL-6 level was significantly higher in patients with metastatic malignant melanoma.

### Multiple Myeloma (MM)

Frassanito et al., 2001 shown increased proportion of T cells producing IL-6 in MM patients with active disease.

Urbanska-Rys et al., 2000 demonstrate a significantly increased serum concentration of IL-6 in MM patients.

Wierzbowska et al., 1999 indicate that serum levels of IL-6 significantly were higher in MM patients, and highest levels were seen in patients with progressive disease.

Pulkki et al., 1996 describe that serum levels of IL-6 and of IL-6R were significantly higher in patients with MM who died within 3 years than in those who survived.

### Ovarian Cancer

Tempfer et al., 1997 describe that median serum level of IL-6 significantly elevated in ovarian cancer patients.

### Prostate Cancer

Nakashima et al., 2000 describe that serum IL-6 levels were significantly correlated with clinical stage of prostate cancer.

Wise et al., demonstrate that serum levels of IL-4, IL-6, and IL-10 were significantly elevated in hormone-refractory prostate cancer.

Adler et al., 1999 shown that levels of IL-6 and transforming growth factor (TGF) correlate with tumor burden and clinically evident metastases.

Drachenberg et al., 1999 describe that serum IL-6 level were significantly elevated in hormone-refractory prostate cancer.

Akimoto et al., 1998 indicate that serum levels of IL-6 related to the metastatic burden to osseous tissue in patients with prostate cancer.

### Renal Cancer

Kallio et al., 2001 describe that serum IL-6 levels prior to surgery were significantly higher in renal cell cancer patients with short survival.

Hayakawa et al., 1998 demonstrate that levels of serum IL-6 and basic fibroblast growth factor were significantly higher in renal cell cancer patients with malignant cysts.

Walther et al., 1998 shown that 56% of patients with metastatic renal cell carcinoma had detectable serum levels of IL-6.

Blay et al., 1997 indicate that serum IL-6 levels were significantly higher in renal cell cancer patients with paraneoplastic fever and weight loss.

Costes et al., 1997 describe that there was a significant difference in survival among renal cell carcinoma patients with detectable levels of IL-6.

Ljungberg et al., 1997 shown that survival time was significantly shorter for renal cell carcinoma patients with serum IL-6 levels above the median level for all patients studied.

### Hepatocarcinoma

Leonardi et al. demonstrate in «the tumor microenvironment in hepatocellular carcinoma» the importance in hepatocellular carcinoma of direct pro-tumoral factor such as e-selectin, HGF, EGF, FGF, MPPs, IL-6, uPA, HGF, TGFβ, IL-6, VEGF, TNF-α, OPN on metastasis, invasion, growth and angiogenesis, and also of indirect pro-tumoral factors such as VEGF, PDGF, TGFβ, IL-8, COX-2, TNF-α, HCV-NSPs, Hbx, MMP9, PDGF, IL-1, M-CSF.

In one embodiment, when used for treating cancer, the first antigen-binding domain of the bispecific antibody binds competitively to TfR1 and the second antigen-binding domain is an antigen-binding domain of the following antibodies: anti-IL-6 antibody, anti-G-CSF, anti-PD-L1 antibody, anti-IL-17B antibody, anti-BAG3 antibody, anti-Gal-3BP antibody, anti-IL-8 antibody, anti-IL-12 antibody, anti-IL-1 antibody, anti-IL-4 antibody, anti-IL-10 antibody,

The bispecific antibody of the present invention can advantageously i) specifically target TfR1 on many kinds of cancers, ii) deprive cells of iron, known for being required in tumors and cancer stem cells growth and progression, iii) significantly accelerate the clearance of soluble pro-tumoral factor from the tumor microenvironment and from the circulation. It is thus a significant and relevant compound for treating cancers by an action on its own by blocking iron uptake in cancer cells, by depleting in a targeted way, protumoral factors in tumors. Both action are potentially synergistic in inducing tumor cell death..

In an embodiment, the format of the bispecific according to the present invention for use in the treatment of cancer is a format with a functional Fc if effector functions are required.

In another embodiment, a mutated Fc or format without Fc can be used if effector functions are not required. A smaller format would allow better tumor penetration.

### Treatment of Inflammatory Pathologies

Furthermore, active inflammatory cells also have transferrin receptor 1. The present invention thus also relates to bispecific antibody for treating inflammatory pathologies.

In another embodiment, the medicament is for the treatment of an inflammatory pathology.

In another embodiment, the present invention also concerns a method for the treatment of an inflammatory pathologies in a subject in need thereof comprising administering to said subject an effective amount of a bispecific antibody as described above.

All the embodiment disclosed above are encompassed in these aspects.

As used herein, “inflammatory disease” or “inflammatory disorder” relates to a complex reaction of vascularized tissue to infection, toxin exposure, or cell injury that involves extravascular accumulation of plasma proteins and leukocytes (Abbas et al., Cellular and Molecular Immunology, 7^{th} edition 2011).

By an “effective amount” of bispecific antibody is meant a sufficient amount to treat inflammatory pathologies, at a reasonable benefit/risk ratio applicable to any medical treatment. It will be understood, however, that the total daily usage of the bispecific antibody will be decided by the attending physician within the scope of sound medical judgment. The specific therapeutically effective dose level for any particular subject in need thereof will depend upon a variety of factors including the stage of the inflammatory pathology and the activity of the specific bispecific antibody employed, the age, body weight, general health, sex and diet of the subject, the time of administration, route of administration, the duration of the treatment; drugs used in combination or coincidental with the and like factors well known in the medical arts. For example, it is well known within the skill of the art to start doses of the compound at levels lower than those required to achieve the desired therapeutic effect and to gradually increase the dosage until the desired effect is achieved.

The term “treatment” or “method of treating” or its equivalent is not intended as an absolute term and, when applied to, for example, inflammatory pathologies refers to a procedure or course of action that is designed to reduce or eliminate or to alleviate one or more symptoms of cancer.

Often, a “treatment” or a “method of treating” inflammatory pathologies will be performed even with a low likelihood of success but is nevertheless deemed to induce an overall beneficial effect. Treatment of inflammatory pathologies refers, for example, to delay of onset, reduced frequency of one or more symptoms, or reduced severity of one or more symptoms associated with the disorder. In some circumstances, the frequency and severity of one or more symptoms is reduced to non-pathological levels.

More particularly, the term of “treatment” or “method of treating” inflammatory pathologies refers to an improvement of clinical behavioral or biological criteria in the subject.

Macrophage can be categorized in 2 subsets: during an immune response to infection, M1 “conventional” macrophages are specialized in acute inflammatory responses and M2 macrophages (or alternatively activated macrophages) are involved in immune response termination and play a role in tissue repair. Both M1 and M2 macrophages express TfR1 but M2 macrophages display high levels of TfR1 compared to M1 macrophages and in the mean time, express low levels of storage protein ferritin (Coma, G et al. Polarization Dictates Iron Handling By Inflammatory And Alternatively Activated Macrophages, Haematologica November 2010 95: 1814-1822).

TfR1 is highly expressed on activated T (Pattanapanyasat, K., and T. G. Hoy. 1991. “Expression of cell surface transferrin receptor and intracellular ferritin after in vitro stimulation of peripheral blood T lymphocytes”. Eur. J. Haematol. 47: 140-145); Yan Zheng, Y et al. A “Role for Mammalian Target of Rapamycin in Regulating T Cell Activation versus Anergy”, J Immunol 2007; 178:2163-2170) and memory B lymphocytes compared to naïve T and B lymphocytes (Paramithiotis, E. and Cooper, M Memory B lymphocytes migrate to bone marrow in humans Proc. Natl. Acad. Sci. USA Vol. 94, pp. 208-212, January 1997).

As immune cells overexpress TfR1, and without wishing to be bound by theory, the bispecific antibody of the invention can specifically target TfR1 on many immune cells and thus being significant for treating inflammatory pathologies.

In another embodiment, the inflammatory pathology is selected from inflammatory bowel disease; psoriasis; rheumatologic inflammatory pathologies such as rheumatoid arthritis, ankylosing spondylitis; multiple sclerosis; autoimmune pathologies such as systemic lupus, erythematosus neuromyelitis optica (NMO); asthma; allergies. Indeed, the following antibodies have been approved in the following indications:

TABLE 12

| Antibody or therapeutic | International | |
|---|---|---|
| molecule | denomination | Indication |
| Anti-TNF-α antibody | Infliximab | inflammatory bowel disease; |
| | | psoriasis; rheumatologic |
| | | inflammatory pathologies; |
| | | multiple sclerosis |
| Anti-TNF-α antibody | Certolizumab | rheumatologic inflammatory |
| | | pathologies, psoriasis |
| Anti-TNF-α antibody | Adalimunab | inflammatory bowel disease; |
| Anti-TNF-α antibody | Golimumab | psoriasis; rheumatologic |
| Anti-soluble TNF-α | Etanercep | inflammatory pathologies |
| receptor | | |
| Anti-IL-6R-antibody | Tocilizumab | rheumatologic inflammatory |
| | | pathologies; NMO |
| Anti-IL-6 antibody | Sirikumab | rheumatoid arthritis |
| Anti-IL-1β antibody | Canakinumab | ankylosing spondylitis |
| Anti-Il-5 antibody | Mepolizumab | Asthma |
| Anti-Il-5 antibody | Reslimumab | |
| Anti-IL-17A antibody | Secukinumab | Psoriasis |
| Anti-IL-17RA antibody | Brodalumab | |
| Anti-IL-17A antibody | Ixekizumab | |
| Anti-sub-unit p40 | Ustekinumab | |
| shared by IL-12 and | | |
| IL23 antibody | | |
| Anti-C5 antibody | Eculizimumab | rheumatologic inflammatory |
| | | pathologies |
| Anti-BAFF antibody | Belimumab | Autoimmune pathologies |
| | | such as systemic lupus, |
| | | inflammatory pathologies |
| Anti-IgE antibody | Omalizumab | allergy |

Hence, and for the treatment of inflammatory diseases, the first antigen-binding domain binds to TfR1 and the second antigen-binding domain is an antigen-binding domain of the following antibodies: anti-TNFα antibody, anti-soluble TNFα receptor, anti-IL-6 antibody, anti-11 antibody, anti-IL-5 antibody, anti-IL-17A antibody, anti-IL12 antibody, anti-IL23 antibody, anti-C5 antibody, anti-BAFF antibody, anti-IgE antibody.

In another embodiment, the format of the bispecific antibody according to the present invention is Fab-ScFV. For the treatment of inflammatory pathologies, effector functions are not required and could even induce toxicity. Hence the Fab-ScFv format is advantageous for the treatment of inflammatory pathologies.

### Route of Administration

The bispecific antibody according to the present invention can be administered y any suitable route of administration. For example, the antagonist according to the invention can be administered by oral (including buccal and sublingual), rectal, nasal, topical, pulmonary, vaginal or parenteral (including intramuscular, intra-arterial, intrathecal, intra-articular, subcutaneous and intravenous) administration of in a form suitable for administration by inhalation or insufflation.

Association with Immune-Check Point Inhibitors

The present invention also concerns a bispecific antibody according to the present invention in association with an immune-checkpoint inhibitor for use in treating cancer.

In another embodiment, the present invention also concerns a bispecific antibody according to the present invention in association with an immune-checkpoint inhibitor for use in treating inflammatory pathologies.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and 25 WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophényl)-N′-hydroxy-4-{[2-(sulfamoylamino)-éthyl]amino}-1,2,5-oxadiazole-3 carboximidamide:

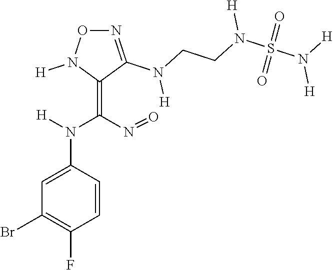 [SMILES: NS(=O)(=O)NCCNC1=NON/C1=C(/N=O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

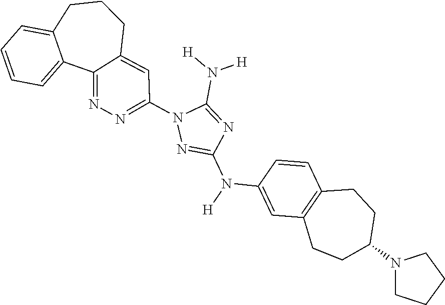 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

Association with Chemotherapy

The present invention also relates to a bispecific antibody according to the present invention in association with a chemotherapeutic agent for use in treating cancer.

In another embodiment, the present invention also relates to a bispecific antibody according to the present invention in association with a chemotherapeutic agent for use in treating inflammatory pathologies.

The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and phannaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and phannaceutically acceptable salts, acids or derivatives of any of the above.

Association with Radiotherapy

The present invention also relates to a bispecific antibody according to the present invention in association with a radiotherapeutic agent for use in treating cancer.

In another embodiment, the present invention also relates to a bispecific antibody according to the present invention in association with a radiotherapeutic agent for use in treating inflammatory pathologies.

The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

### EXAMPLES

### Material and Methods

(scFv)₂-Fc and Full Length IgG1 Antibody Design and Production

To obtain scFv fused with the Fc fragment of human IgG1, the cDNA encoding the six anti-TfR1 scFv antibodies or anti-botulinum toxin (negative control) were NcoI/NotI-digested from the phagemid pHEN and subcloned into the pFUSE-hFc2(IL2ss) vector, a gift from Frank Perez, CNRS-Institut Curie, Paris, France. Soluble 100 kD (scFv)₂-Fc antibodies were produced after transient transfection of HEK-293 T cells and purified using protein. Full length H7-IgG1 and 2D1-IgG1 (anti-CD117 antibody)⁴ were produced in CHO cells from their VH and VL sequences by EVITRIA (EVITRIA, Switzerland). C32-IgG1, F2-IgG1, H9-IgG1, G9-IgG1, H7-IgG1 Del297 (that has reduced affinity for Fcγ receptors due to deletion of the Asn297 residue) and the anti-TfR1 mouse mAb Ba120⁶ were a gift from Alexandre Fontayne (LFB, France). The apparent affinities for TfR1 of the two H7-IgG1 and H7-IgG1 Del297 were identical (not shown). Rituximab was from Roche. The irrelevant IgG1 antibody was a human polyclonal IgG (SIGMA, I2511). Antibody concentrations were verified by measuring their A₂₈₀ₙₘ by spectrophotometry (1 UA at 280 nm corresponds to 0.8 mg/mL) and purity was checked by SDS-PAGE.

### Cell Lines

The B-cell lymphoma Bp3, Im9 (a gift from Nathalie Bonnefoy, IRCM) and RAJI cell lines, the erythroleukemia ERY-1 cell line (a gift from Michel Arock, LBPA, ENS Cachan, France), and the BxPC3 and CFPAC pancreatic cancer cell lines (obtained from ATCC) were grown in RPMI-Glutamax supplemented with 10% fetal bovine serum (FBS; ThermoScientific SV30160.03) and with penicillin/streptomycin (Gibco 15240-062). The mouse P815 (a gift from Nicolas Fazilleau, Institut Pasteur, Paris, France) and human HMC11 (a gift from Michel Arock, ENS Cachan, France) mastocytoma cell lines were grown in IMDM. Adherent HEK-293 T cells (a gift from Laurent Le Cam, IRCM) were grown in DMEM, 10% FBS and antibiotics. All cell lines were cultured at 37° C. in a humidified atmosphere with 5% C02 and screened monthly for mycoplasma infection.

### Commercial Antibodies and Reagents for FACS, Western Blotting and IHC

Anti-human CD71 (Invitrogen, 136800), -HIF1-α (Santa Cruz Biotechnology, sc-8711), and -beta-actin (Cell Signaling Technology 3700S) antibodies were used for western blotting. PE-conjugated anti-mouse TfR1 (BD Pharmingen, 553267), APC-conjugated anti-human TfR1 (BD Pharmingen 551374), FITC-conjugated goat anti-human Fc (SIGMA, F9512) or anti-mouse Fc (Invitrogen, 31569) antibodies were used for FACS analysis of TfR1 levels. Anti-human TfR1 (SIGMA, HPA028598) was used for IHC. Human holo-Tf was from SIGMA (T0665), DFO from Santa Cruz (Sc-203331; stock solution: 50 mM in H20, stored at 4° C.), holo-Tf conjugated to Alexa Fluor 488 (holo-Tf-A488) from Invitrogen (T13342; 50 μM solution in PBS stored at 4° C.), and calcein-AM from Invitrogen (C3100MP; stock solution: 50 μM in DMSO at −20° C.).

### Holo-Tf Uptake Measurement

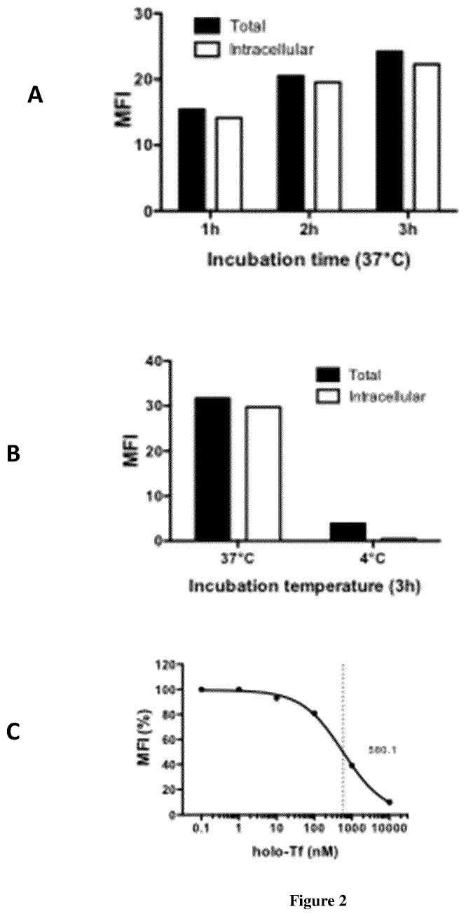

Raji cells (5×105) were washed and resuspended in RPMI medium supplemented with 1% fetal calf serum (FCS) and 500 nM holo-Tf-A488 together or not with antibodies or non-conjugated holo-Tf at 37° C. for 3 h. Cells were then washed with cold PBS and the cell fluorescence associated with holo-Tf-A488 uptake measured by FACS (FC500 cytometer, Beckman Coulter). Preliminary experiments with an additional incubation of cells with 50 mM glycine pH 2.8/500 mM NaCl buffer for 10 min at 4° C. before FACS analysis showed that the fluorescence measured was more than 95% intracellular. Therefore, this step was omitted in further experiments to limit the steps before analysis (FIG. 2B). The cell mean fluorescence intensity (MFI) was calculated using the Flow Jo Version 10.1r7 software.

### Antibody Apparent Affinity and Antibody/Ligand Competition

All incubation steps were done on ice. Raji cells (5×105 cells) were resuspended in 100 μL of FACS buffer [PBS, 1% FBS] containing various concentrations of the primary antibody for 1 h, washed twice with FACS buffer, and then incubated with the suitable fluorescent secondary antibody. After a final wash, cells were analyzed by FACS. The apparent affinity was determined using the GraphPad software. For competition experiments, cells were incubated with 1 nM antibody mixed with increasing concentrations of holo-Tf (0.5 pM to 5 μM). Then, bound antibodies were detected as before. Alternatively, cells were incubated with 500 nM holo-Tf-A488 mixed with increasing concentrations of antibody (5 nM to 500 nM), or with holo-Tf (0.1 nM to 10 μM).

### Intracellular Free Iron Detection

Intracellular free iron levels were measured using the fluorescent probe calcein, as previously described ref. 10.10. This probe binds to iron stoichiometrically in a reversible manner, forming fluorescence-quenched calcein-iron complexes. Therefore, higher cell fluorescence means that the levels of intracellular labile iron pool are reduced. Briefly, Raji cells were washed and resuspended in medium without FCS and stained with 250 nM calcein-AM at 37° C. for 5 min. They were washed with complete medium and resuspended in pre-warmed culture medium with 1% FCS and incubated with the studied antibodies or deferoxamine (DFO) for 4 or 8 h. Cell fluorescence due to free calcein was measured by flow cytometry and the percentage increase in calcein fluorescence relative to untreated control was calculated.

### Western Blotting

Raji cells (5.105 cells in 2 mL) were incubated with holo-Tf or antibodies for 1.5 or 3 days. Cells were harvested, centrifuged and washed with cold PBS. Proteins were extracted with 100 μL of boiling lysis buffer (1% SDS, 1 mM sodium orthovanadate, 10 mM Tris pH 7.4)/cell pellet. The viscous mix was sonicated on ice four times at 25 mA for 5 s, and then centrifuged. Protein concentration was determined using the BCA assay (Interchim). Proteins were extracted from tumor samples using a lysis buffer containing 1% Triton-X100, 0.5% NP40, 1 mM EDTA, 150 mM NaCl, 10 mM Tris-Hcl pH 7.5, 100 mM NaF, 1 mM sodium orthovanadate, 2 mM phenylmethylsulfonyl fluoride complemented with 1 tablet of protease inhibitors mixture for 10 mL (Roche Diagnostics). A piece of tumor of 10 mm3 was cut into small pieces, and then 0.5 mL of lysis buffer was added at 4° C. for 30 min, followed by grinding with glass beads using a Retsch MM300 TissueLyser (Qiagen) at maximum power for 3 min, followed by incubation at 4° C. for 30 min. Protein extracts were centrifuged (12,000 g at 4° C. for 30 min), and proteins in the soluble fraction quantified with the BCA assay. For western blotting, 20 μg of protein were separated by SDS-PAGE on 7% polyacrylamide separation gels and transferred to PVDF membranes. Membranes were blocked with PBS/0.1% Tween-5% milk at room temperature (RT) for 2 h. Incubations with primary and secondary antibodies were done overnight at 4° C. and 1 h at RT, respectively. Membranes were revealed with Western Lightning PLUS-ECL (Perkin Elmer) and analyzed with a G-box (Syngene).

Determination of scFv2-Fc Concentration in Serum Samples by ELISA

Blood samples were centrifuged at 1500 g for 15 min, and serum samples were stored at −20° C. until dilution (1000 times in PBS) and scFv2-Fc titration by ELISA. An ELISA sandwich assay (linear range from 10 to 150 ng/mL) was specifically developed using a goat anti-human Fc as the capture antibody (Sigma, I-2126, 10 μg/mL) and a HRP-conjugated goat anti-human-Fc antibody as the detection antibody (A0170, dilution 20 000). Samples were tested in duplicate and tittered in two independent ELISA experiments.

### Pharmacokinetics

Ten wild type (WT) C57Bl/6 (Janvier, Saint-Berthevin, France) and FcRnKO (B6.Cg-Fcgrt^{tmlDcr}) (Jackson Laboratory, Bar Harbor, Me.) mice received an i.v. retro-orbital injection of 80 μg of scFv₂-Fc (single dose). From 2 h to day 21 post-injection, blood samples were collected and scFv₂-Fc tittered by ELISA. A two-compartment model was designed to describe cellular uptake of antibodies. Compartments were serum (S) and intra-cellular (C) and k_{SC}, k_{CS} and k_{E} are cellular uptake, FcRn recycling and intra-cellular elimination rate constants, respectively. The pharmacokinetics of antibodies was analyzed using population pharmacokinetic modelling using Monolix®2018 suite (Lixoft, Orsay, France). Interindividual and residual variabilities of the pharmacokinetic parameters were estimated using exponential and proportional models, respectively. The association of FcRn (WT vs. KO) and antibody (Irr-Fc vs H7-Fc) factors was tested as dichotomous covariates on pharmacokinetic parameter interindividual distributions. These covariates were tested using likelihood ratio tests (LRT) based on objective function value (OFV). From pairs of nested models (i.e. models with vs. without covariate), the difference between their OFV was tested using a chi-square test. A covariate was considered as significant if corresponding p-value was <0.05

### Statistical Analysis

A linear mixed regression model was used to determine the relationship between tumor growth and the number of days post-graft. The fixed part of the model included variables corresponding to the number of days post-graft and the different groups. Interaction terms were built into the model. Random intercept and random slope were included to take into account the time effect. The coefficients of the model were estimated by maximum likelihood and considered significant at the 0.05 level. Survival rates were estimated using the Kaplan-Meier method from the date of the xenograft until the date when the tumor reached a volume of 1,600 mm3. Survival curves were compared using the log-rank test. Statistical analyses were carried out using the STATA 11.0 software (StataCorp, College Station, Tex.).

### Example 1: Antibody Binding to TfR1 and Inhibition of Holo-Tf Internalization

TfR1 is the main receptor responsible for the cell iron supply through receptor-mediated internalization of serum Fe³⁺-loaded transferrin (holo-Tf). Within the cell, Fe3+ is released, reduced, excluded from the early endosome by divalent metal ion transporter 1 (DMT1), and used for cell metabolism. Fe3+ in excess is stored in ferritin, while TfR1 is recycled at the cell surface together with iron-free transferrin (apo-Tf).

The anti-TfR antibodies were obtained in vitro by phage display in an scFv format (single chain fragment variable format) and were selected for their capacity for being endocyted (internalized) specifically inside mammary tumour cells (SKBR3).

This selection enabled 6 antibodies each of molecular weight 28 kDa to be obtained, called respectively F12 (of SEQ ID NO:7), F2 (of SEQ ID NO:25), H9 (of SEQ ID NO:40), C32 (of SEQ ID NO:10), G9 (of SEQ ID NO:55) and H7 (of SEQ ID NO:4).

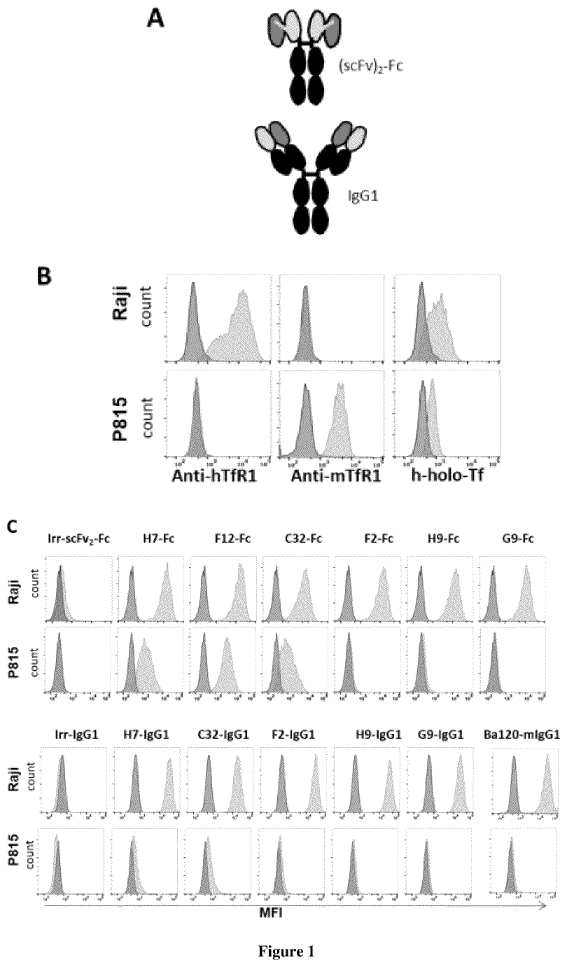

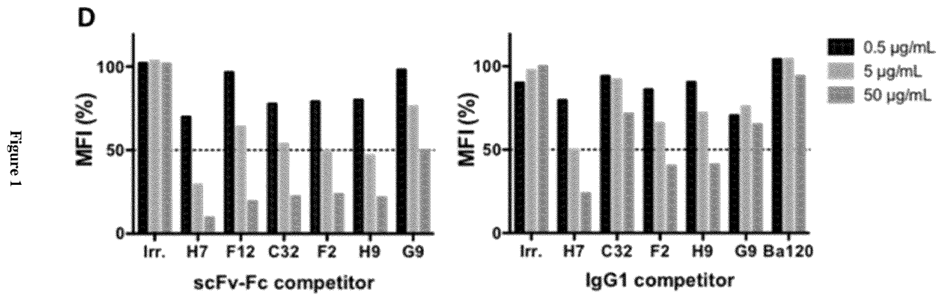

All the six parental anti-TfR1 scFv antibodies (H7, F12, C32, F2, H9, G9) could be converted into the scFv2-Fc and IgG1 antibody formats (FIG. 1A), with high production yields for F12-IgG1.

The initial characterization was done to verify that the new antibody formats could bind to TfR1 and inhibit TfR1-mediated holo-Tf internalization, like the parental scFv antibodies. The TfR1-expressing B-cell lymphoma Raji and mastocytoma P815 cell lines were used to test the binding to human and mouse TfR1, respectively (FIG. 1C). Among the scFv2-Fc antibodies, only H7, F12 and C32 recognized also mouse TfR1 (FIG. 1 C upper panel). Among the IgG1 antibodies, H7 and C32 lost cross-reactivity to mouse TfR1 (FIG. 1 C lower panel). All six scFv2-Fc antibodies inhibited internalization of Alexa 488-conjugated holo-Tf (holo-Tf-A488), and H7-Fc was the most efficient with 70% inhibition at 5 μg/mL (50 nM) (FIG. 1 D). Concerning the IgG1 antibodies, the inhibition of holo-Tf internalization by G9 and C32 was greatly reduced compared with the scFv2-Fc format. H7-IgG1 was again the most efficient with 50% inhibition at 5 μg/mL (33 nM). Another anti-TfR1 mAb Ba120 (mouse IgG1), which shows inhibitory activity in leukemia models, 18 had not effect.

H7 was then chosen for more extensive characterization and comparison with Ba120.

To test their capacity to block internalization of holo-Tf at physiological concentrations, 5 μg/mL of H7-Fc and H7-IgG1 (i.e., 50 nM and 33 nM, respectively) were mixed with 10 μM holo-Tf-A488.

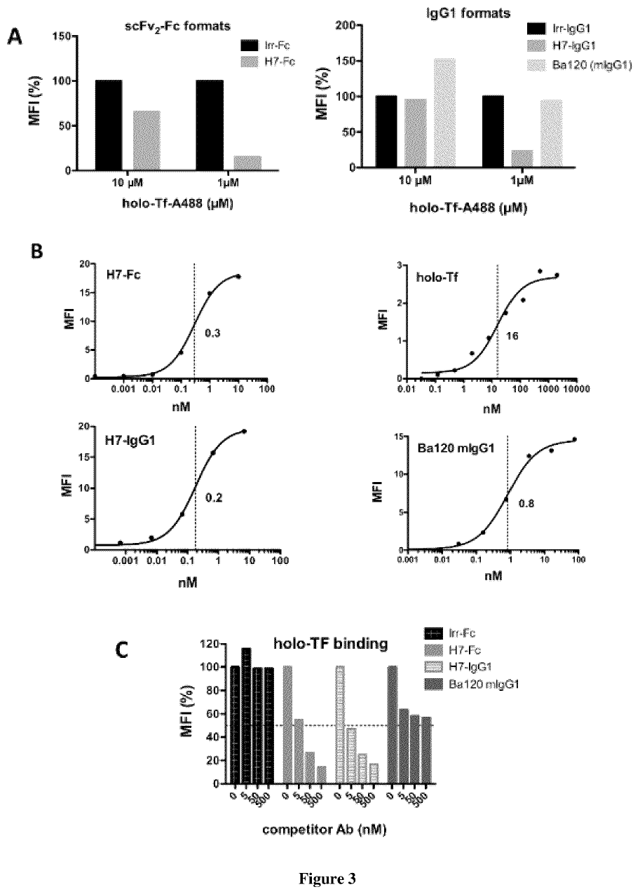

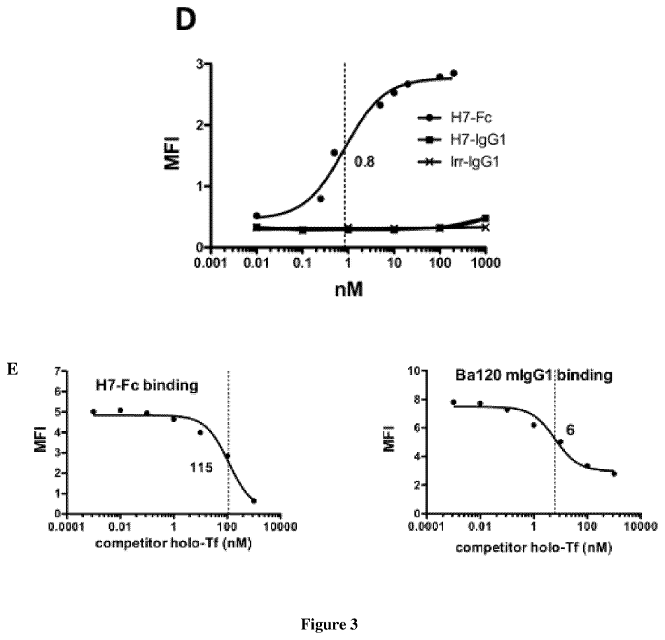

H7-Fc, but not H7-IgG1, still inhibited holo-Tf internalization in Raji cells [FIG. 3A left panel and right panel respectively]. Surprisingly, Ba120 increased holo-Tf internalization of more than 50%. The apparent affinity constant (EC50) [FIG. 3B] and the antibody concentration that blocked 50% of holo-Tf-A488 binding (used at 500 nM) to human TfR1 (IC50) at 4° C. in Raji cells [FIG. 3C] were then determined. H7-Fc, H7-IgG1 and Ba120 displayed subnanomolar EC₅₀ values showing better binding to human TfR1 than holo-Tf (EC₅₀ 16 nM) in the same conditions. Moreover, H7-Fc and H7-IgG1 fully inhibited holo-Tf binding to TfR1 (IC₅₀ of 5 nM), whereas Ba120 could only inhibit 50% of binding (FIG. 3C), consistent with Ba120 inability to reduce holo-Tf internalization (FIGS. 1D and 3A). When measured on mouse TfR1 using the p815 mouse cell line, H7-Fc displayed an EC50 of 0.8 nM (FIG. 3E), in the same range as the EC50 for human TfR1 measured in Raji cells (0.3 nM). Finally, analysis of antibody (1 nM) binding in the presence of increasing concentrations of holo-Tf at 4° C. showed that in Raji cells, H7-Fc binding to TfR1 could be fully inhibited (IC50 115 nM). Conversely, Ba120 binding was inhibited only by 50% even in the presence of a 1000 molar excess of holo-Tf [FIG. 3E].

Altogether, these results indicate a competitive inhibition of holo-Tf binding by H7 (i.e., the H7 epitope on TfR1 overlaps with the holo-Tf binding site). Molecular modeling confirmed the interaction of H7 at the site of holo-Tf binding on TfR1, and showed that the Ba120 epitope was away from the holo-Tf binding site on TfR1 (data not shown).

### Example 2: H7 Fate Upon TfR1 Binding

Upon its natural ligang holo-Tf binding, TfR1 is rapidly internalized and recycled after holo-Tf has released iron in the endosomes. In physiological conditions, TfR1 expression depends on LIP level through the regulation of TfR1 mRNA stability.

Previously described anti-TfR1 competitive inhibitory antibodies decrease TfR1 level through antibody-dependent TfR1 routing to the lysosome where it is degraded. Degradation of TfR1 upon non ligand competitive anti-TfR1 antibody has been shown to be enhanced by high affinity and/or dimeric receptor binding compared to lower affinity and/or monomeric binding of TfR1

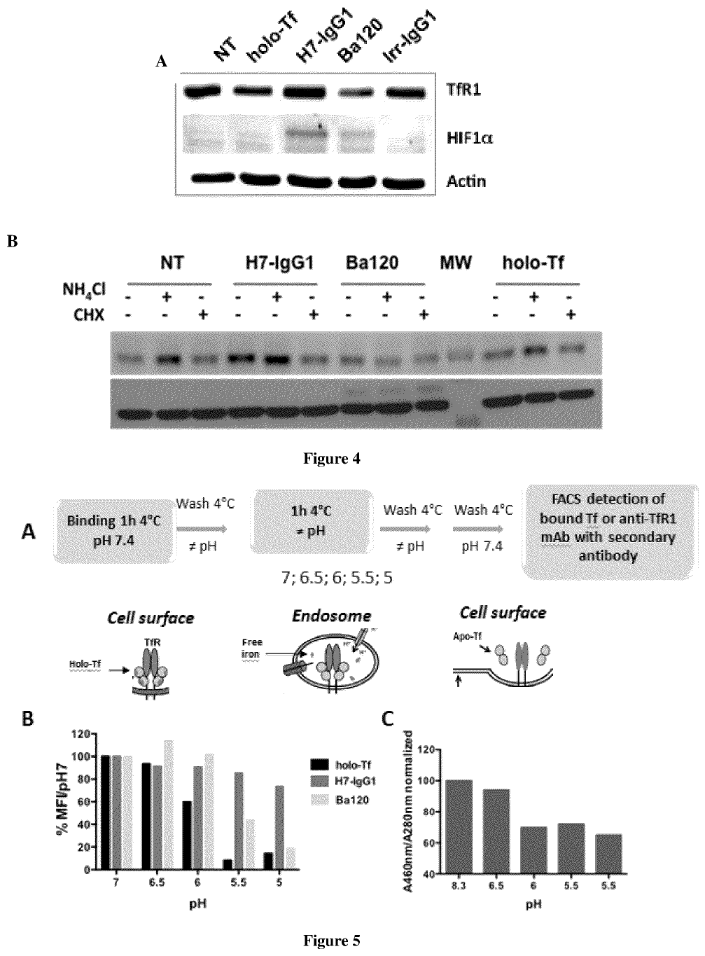

Here, incubation of Raji cells with the high affinity bivalent anti-TfR1 H7 (5 μg/mL) for 36 h led to TfR1 level increase. Hypoxia inducible factor 1-alpha (HIF-1α), the stability of which is affected by LIP through iron dependent proteases was also strongly increased by H7 treatment (FIG. 4A). TfR1 increase upon treatment was strongly prevented by translation inhibition by cycloheximide and slightly increased by NH₄Cl treatment that limits lysosome acidification (FIG. 4B). Conversely, holo-Tf and Ba120 treatment reduced TfR1 level after 36 h of treatment (FIG. 4A). These data suggest that unlike Ba120, H7 does not interfere with TfR1 recycling and induces limited TfR1 degradation.

Finally, H7 binding to TfR1 was not decreased at pH 6 compared with pH 7 (FIGS. 5 A, B and C), indicating that, like apo-Tf, H7 might not be released in the endosome and could be recycled back to the cell surface together with TfR1. However, unlike apo-Tf, which has reduced affinity for TfR1 at extracellular pH, H7 should not dissociate at the cell surface and therefore, reduce strongly the accessibility of the recycled TfR1 to iron charged holo-Tf, thus explaining H7 high iron deprivation efficiency.

To explore the potential consequences of the TfR1 modulation by H7 observed in vitro on H7 pharmacokinetics/pharmacodynamics, the biodistribution of a mixture of ¹²⁵I-labeled H7-Fc and ¹³¹I-labeled irrelevant scFv2-Fc antibodies was evaluated in mice. The scFv2-Fc format was chosen because, differently from the IgG1 format, it can cross-reacts with mouse TfR1 (FIG. 1C). Nude mice bearing s.c. ERY-1 tumor cell xenografts received one (i.v.) injection of the two antibody mixture (6 μg, 5 μCi/each) (n=4 mice/group). The percentage of the injected dose (% ID) after 48 h was similar for H7-Fc and the irrelevant scFv₂-Fc antibody, consistent with the well described enhanced permeability and retention (EPR) effect in tumors.

Individual variations among animals could be explained by the different tumor sizes (300 to 800 mm3). However, as indicated by the organ repartition index, H7-Fc specificity for mouse TfR1 resulted in increased radioactivity associated with the tumor compared with the irrelevant scFv2-Fc antibody (ratio>1). In a parallel experiment, titration by ELISA of the serum H7-Fc after one single i.v. injection (80 μg) in C57Bl/6 mice (WT) and C57Bl/6 FcRnKO mice showed that in WT mice, H7-Fc was cleared from the serum more rapidly than the irrelevant scFv2-Fc. A two-compartment model was designed to describe the cellular uptake of antibodies. In this model, the apparent distribution volume (VD) of H7-Fc was higher than the VD of Irr-scFv2-Fc, both in WT and FcRn KO mice, consistent with intracellular localization of H7-Fc (2.5 mL versus 1 mL for the H7-Fc and Irr-Fc, respectively, (LRT, p<0.0005)). As expected, recycling due to FcRn, represented by the kCS rate constant, was decreased in FcRn KO mice for both antibodies and Irr-Fc elimination half-life decreased in FcRn KO mice compared to WT mice (7.9 to 1.7 days, LRT, p<0.0005), However, strikingly, H7-Fc elimination half-life was not impacted by the FcRn KO background and remained around 5 days. Of note, since both FcRn and antibody factors were quantified simultaneously in the multivariate model, the effects due to FcRn and TfR1 binding and recycling are independent. Altogether, the biodistribution and PK results indicate a dominant target mediated stabilization mechanism for H7-Fc.

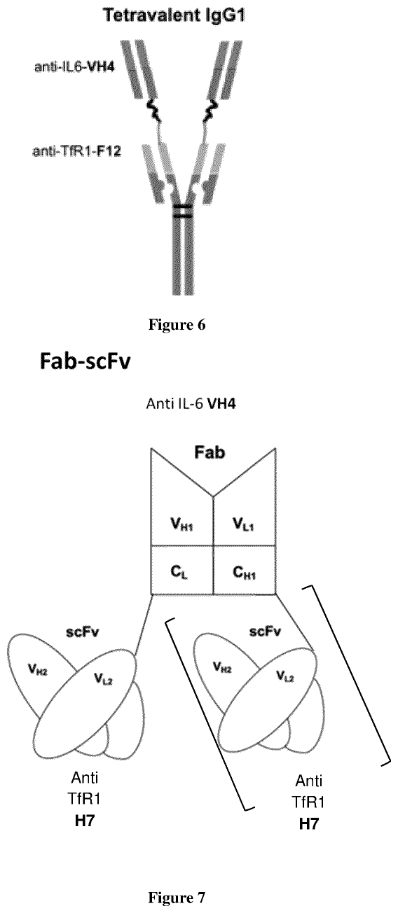

Finally, nude mice with established s.c. ERY-1 tumor cell xenografts were treated with H7-Fc (5 mg/kg i.p. twice a week) or PBS (n=5 animals/group). After 4 weeks of treatment, two animals were cured in the H7-Fc group. Moreover, western blot analysis of the tumors (FIG. 6G) showed that TfR1 levels were increased in the tumors of the other three mice treated with H7-Fc compared with the tumors of the PBS group. IHC analysis of one tumor for each group (900 mm³) with an anti-TfR1 antibody showed higher TfR1 staining in the H7-Fc treated sample (data not shown). These data indicate that H7-Fc treatment upregulates TfR1 in vivo, as observed in vitro, suggesting that tumors treated with H7 undergo iron deprivation.

### Conclusions

Starting from a panel of anti-TfR1 scFv antibodies that were isolated for their rapid cell internalization upon antigen binding, bivalent antibodies harboring a human Fcγ1 were engineered. It has been found that for the scFv2-Fcγ1 format, H7-Fc was the most efficient antibody concerning inhibition of holo-Tf uptake. This was due to H7 great efficiency in blocking holo-Tf binding (2 log lower molar concentrations of H7 are required to block holo-Tf binding, and 2 log higher molar concentrations of holo-Tf are required to block H7 binding). H7-IgG1 maintained this feature, but lost cross-reactivity to mouse TfR1.

Examples 1 and 2 demonstrates that incubation of cells with H7-Fc or H7-IgG1 increased TfR1 level similarly to incubation with the 50 kD dimeric (scFv)2 H7 antibody (H7-scFv2). Therefore, the presence of an Fc region did not change the receptor modulation. This property is unique because other previously described high affinity anti-TfR1 antibodies decrease TfR1 level through traffic diversion and degradation within lysosomes. Thus, TfR1 normal trafficking seems not to be diverted by H7 binding. Combined with the efficient iron deprivation that promotes TfR1 translation this property contributes to the TfR1 level increase observed in vitro and in vivo upon H7 treatment.

H7-mediated iron deprivation is comparable to the one obtained with the iron chelator DFO and higher than with Ba120. The inventors also find that Ba120 increases rapidly soluble iron levels in Bp3 and Im9 cells-lines. Because Ba120 induces TfR1 degradation, visible after 36 h in Raji cells, the increase in soluble iron level mediated by Ba120 is probably only transient.

As H7 binds with similar affinity to TfR1 at extracellular and endosomal pH, H7 may be recycled at the cell surface with the receptor after it has induced its internalization, thus immediately preventing TfR1 association with extracellular holo-Tf.

The increased efficiency (>2 log) of ERY-1 cell viability inhibition by the bivalent H7-IgG1 (IC₅₀ 0.5 nM), H7-Fc (IC₅₀ 1.4 nM) and H7-scFv₂ (IC₅₀ 2 nM) compared with the monovalent H7-scFv (IC₅₀ 200 nM) suggests that these bivalent antibodies can bind to two proximal TfR1 receptors on cells in which the receptor is present at high density. Accordingly, lower toxicity is expected in cells that express low level of TfR1, as previously suggested for the anti-TfR1 mAb A24 and demonstrated for the anti-TfR1 JST-TFR09 antibody. In agreement, no obvious toxicity was observed in mice treated with H7-Fc (cross-reactive with mouse TfR1) for 1 month compared with untreated mice (PBS), indicating that despite background TfR1 expression in many tissues, iron deprivation due to H7 should have limited toxicity in vivo. However, since non-competitive effector competent anti-TfR1 antibodies have been shown to transiently elicit acute clinical signs and to clear immature blood reticulocytes in mice it is not excluded that such a toxicity may occur with the competitive anti-TfR1 H7 of this study. This will be to determine using an effector function competent variant of scFv₂-Fc H7 in mice and ultimately using the non-cross reactive H7-IgG1 in a non-human primate model.

As TfR1 is expressed at low level by many cell types, it was hypothesized that antigen-dependent recycling of H7 could protect this antibody from degradation, in an FcRn-like process. Indeed, FcRn and TfR1 share similar intracellular trafficking and both can rescue their respective ligands from lysosomal degradation.

To test this hypothesis, the clearance of the cross-reactive H7-Fc in WT and FcRnKO mice have been compared because human IgG1 binding to mouse FcRn receptors allows relevant PK observations in mice. In FcRnKO mice, H7-Fc elimination half-life was only weakly affected compared with WT mice, while the elimination half-life of an irrelevant scFv2-Fc antibody was dramatically reduced, as previously reported for this antibody format.

This observation could also explain the antitumor effect of H7-scFv2 in nude mice harboring s.c. ERY-1 tumors, although no therapeutic effect was expected because of its small size (50 kDa) and fast serum clearance (data not shown). H7 specificity and its unique mode of interaction with TfR1 (it acts like an exact mimic of the natural ligand) increase its persistence in vivo through an FcRn-like mechanism that is independent of the Fc part of the antibody.

### Example 3: Production of Bispecific Format F12-VH4

Starting from the monospecific format F12 (anti-TfR1) (of SEQ ID NO:7) and from the monospecific VH4 (anti-IL6) as described in Devanaboyina and al. a tetravalent bispecific antibody BsAb anti-TfR1-anti-IL-6 with pH dependent binding was produced in insect cells as described in Golay J. et al. J. Immunol. 2016) (FIG. 6). In this format, the VH-CH1-hinge domains of VH4 are fused through a peptidic linker to the N terminus of F12 H chain, and paired mutations at the CH1-CL interface of VH4 are introduced that force the correct pairing of the two different free L chains Monoclonal IgG1 anti-TfR1 and IgG1 anti-IL-6 were also produced as a control. One mg of each molecule was provided.

This format contains mutations as summarized in the table hereinafter.

| Mutations | Region | Effect |
|---|---|---|
| T192E | CH1 (Fab position 2) | Controlling the correct pairing of |
| N137K | CH1 (Fab position 2) | the L chain with its corresponding |
| S114A | CL (Fab position 2) | H chain |

In these examples, the terms “BsAb”, “tetraBsAb”, “IgG1 tetravalent” relates to the tetravalent bispecific antibody BsAb anti-TfR1-anti-IL-6 with pH dependent binding.

### Example 4: Production of Bispecific Format Fab-scFv

Another format was specifically designed devoided of Fc region for better tumor penetration and/or lower avidity for TfR1 and reduced ADCC activity to lower potential toxic effect (FIG. 7), starting from the monospecific format H7 (anti-TfR1) (of SEQ ID NO:4) and from the monospecific VH4 (anti-IL6).

A control antibody BE8 (mouse monoclonal anti-IL6, neutralizing antibody was provided by Jérôme Moreaux, IGH, Montpellier, Clin Cancer Res. 2003 October; 9(13): 4653-4665.

### Example 5: Production of Bispecific Format scFv-Fc

Another format comprising a Fc region was designed (FIG. 16). The Fc region of the scFv-Fc format is modified to favour heterodimerization but is devoided of effector functions.

scFv-Fc format contains mutations as summarized in the table hereinafter.

| Mutations | Region | Effect |
|---|---|---|
| L234A, L235A, P329G | CH2 | No ADCC, no CDC |
| S354C, T366W (chain A) | CH3 | Knob into Hole, bispecific |
| Y349C, T366S, L368A, | CH3 | association favoured |
| Y407V (chain B) | | |

### Example 6: Molecular Characterization of Bispecific Antibody F12-VH4 (BsAb)

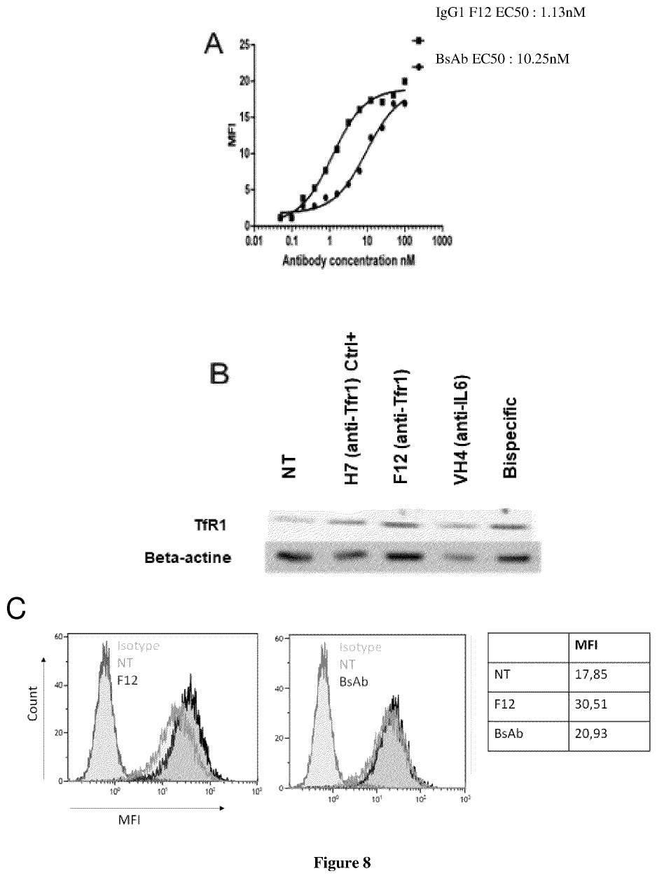

The binding of the bispecific antibody BsAb anti-TfR1-anti-IL-6 with pH dependent binding (hereinafter “BsAb”) on cells (B lymphoma Raji cells, non IL-6 producing cell line) was confirmed. However, monovalent anti-TfR1 F12 binding to TfR1 was better compared to BsAb (EC50 1 nM and 10 nM, respectively) (FIG. 8A) likely due steric hindrance, a consequence of the position chosen for the anti-TfR1 moiety (see FIG. 6).

BsAb 48 h. treatment of BxPC3 pancreatic cancer cell line upregulated both TfR1 total levels (FIG. 8B) and cell surface levels (FIG. 8C).

Additionally, BsAb binding to TfR1 was blocked by an excess of holo-Transferrin and if incubated at 37° C. for 90 min, BsAb was readily internalized in the CFPAC pancreatic cell line (data not shown). For this experiment, CFPAC cell line, that secretes IL-6, are plated on coverslips, grown 3 days, then incubated with BsAb (30 μg/mL) at 4° C. or 37° C. for 90 min. Cells are fixed 40 min. with formalin at RT, BsAb is then detected with a anti-Hu-Fc conjugated with FITC. Alternatively, holo-Tf (10 μM) is added at the same time than the BsAb. This indicates that the BsAb has similar trafficking pathway compared to the parental F12 antibody, at least in absence of IL-6.

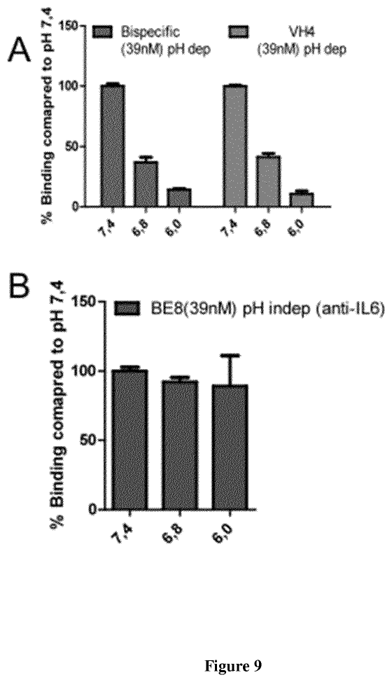

The binding of BsAb to IL-6 was tested on recombinant IL-6 by ELISA (FIG. 9). BsAb only bound to IL-6 at physiological pH but not at acidic pH, like the parental anti-IL-6 VH4 (FIG. 9A), as opposed to BE8 binding to IL-6, which is not affected by the pH (FIG. 9B). This pH-dependent binding will allow the release of IL-6 in acidificating endosomes.

The ability of the BsAb format to bind endogenous IL-6 was further tested on the IL-6 producing CFPAC cell line.

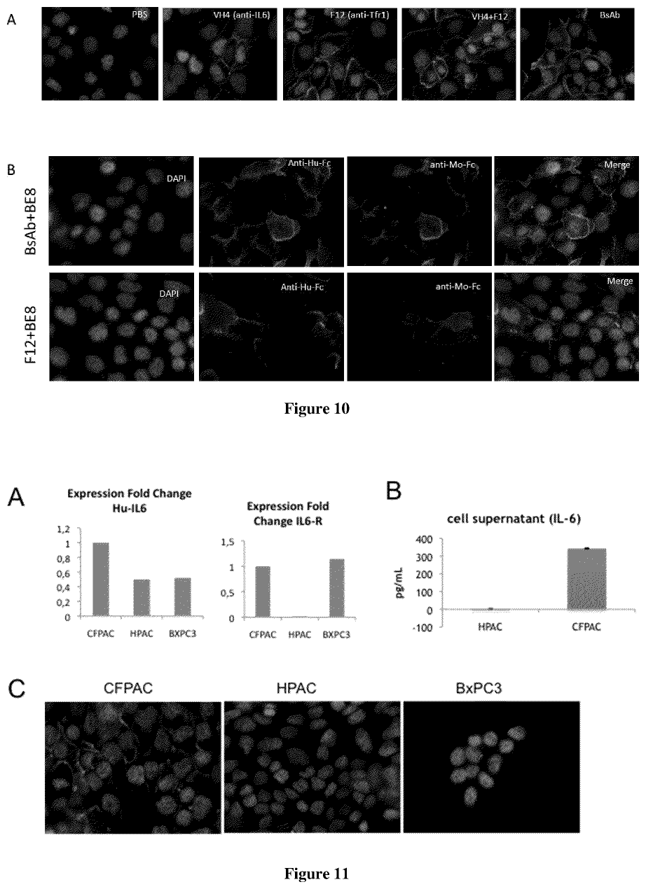

CFPAC were grown for 2 days on cover slips then transferred at 4° C. and incubated with the BsAb (30 μg/mL) for 90 min. Cells were then fixed and permeabilized using formalin and BsAb was detected with an anti-Hu-FITC. The pattern of staining observed (cell surface staining and extracellular staining) was similar to the one observed when cells were incubated with a mix of anti-IL6 VH4 and anti-TfR1 F12 parental antibodies (both Igg1 isotype) suggesting that the BsAB is able to bind to both antigens (FIG. 10A). IL-6, as detected with the non VH4 competing mouse monoclonal anti-IL6 antibody BE8, colocalized with the BsAb at the cell membrane (FIG. 10B, upper panel). In contrast, TfR1 and IL-6 co-localization was not observed when CFPAC cells were treated with the parental anti-TfR1 F12 antibody (FIG. 10B, lower panel). This confirms that the tetravalent BsAb used can bind simultaneously IL-6 and TfR1.

Altogether these data show that the anti-IL6-anti-TfR1 BsAb is able to bind endogenous IL-6 and TfR1 and to internalize into TfR1 positive cells CFPAC.

The binding of the scFv-Fab and (scFv)2-Fab anti-IL-6 produced in HEK cells to TfR1 on Raji cells was confirmed by FACS using crude HEK supernatant followed by anti-human CK-FITC antibody. The pH dependent binding to IL-6 was also confirmed by ELISA (data not shown).

### Example 7: Sweeping Effect

### Cell Lines Characterization

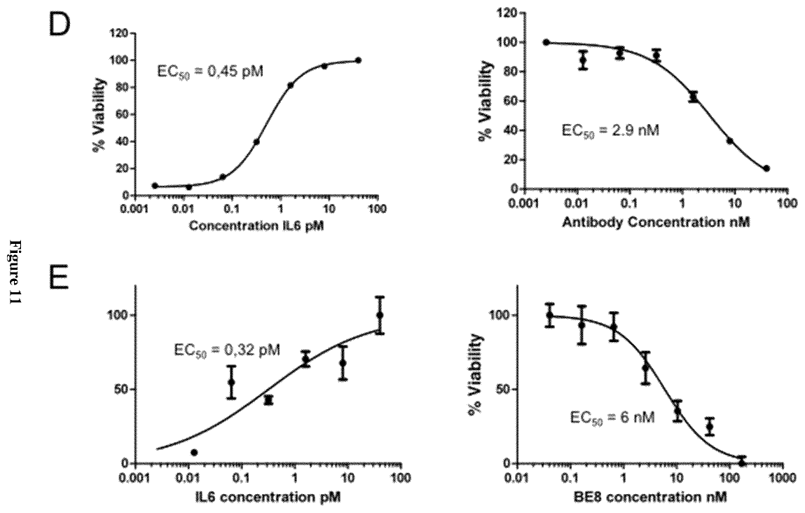

Pancreatic cancer cell lines were tested by RT-PCR for the expression of IL-6 and IL6-R by PCR (FIG. 11A) and the accumulation of IL6 in cell supernatant by ELISA (FIG. 11B) or in the extracellular matrix by immunofluorescence (IF) (FIG. 11C). As published by others, CFPAC, HPAC and BxPC3 were found to express IL-6 mRNA. Only CFPAC expressed IL-6 at detectable levels by ELISA and IF. It has been previously shown that all three cell line express TfR1.

The XG6 and XG7 myeloma cell lines were dependent of IL-6 for their growth (FIG. 11D) as previously published. The EC50 (concentration of IL-6 allowing 50% of maximum growth) was determined to be 0.4 pM for both cell lines. When cells were grown in the presence of 40 pM of IL-6, the neutralizing anti-IL-6 BE8 inhibited XG6 and XG7 growth (IC50 of 3 and 6 nM, respectively) (FIG. 11E).

The MCF7 line was used as a non IL-6, non IL-6R expressing cell line as published by others (Zhong et al., 2016).

The Bispecific Antibody Induces TfR1 Dependent IL-6 Uptake into the MCF7 Cell Line

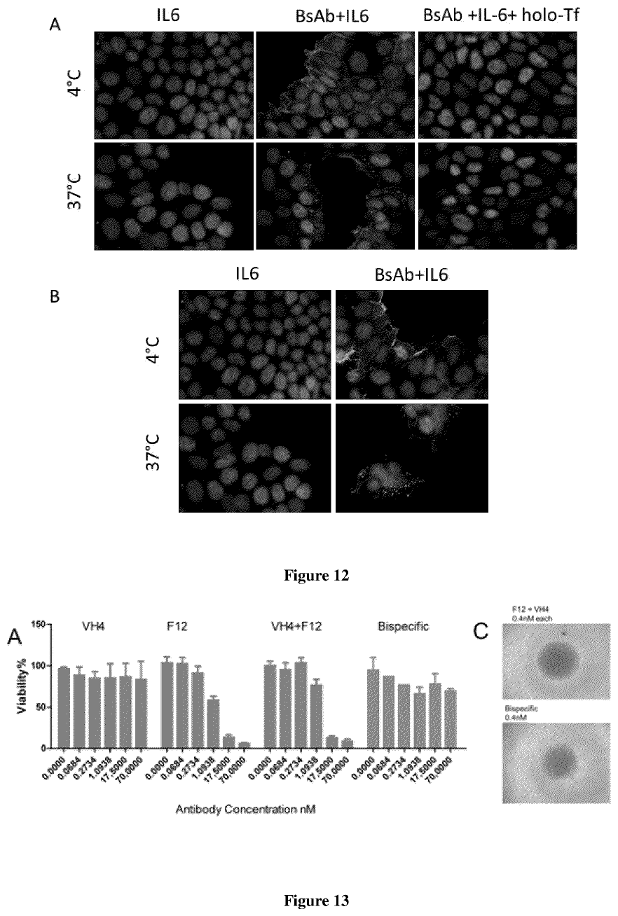

The ability of the bispecific antibody (BsAb) to induce IL-6 cell uptake through TfR1 mediated endocytosis was tested on the non IL-6, and non IL6R expressing cell line. MCF7 cells were incubated for 1 hour. in the presence of IL-6 and BsAb at 37° C. Cells were then fixed and permeabilized and IL-6 detected with the anti-IL6 BE8 antibody. While in MCF7 treated with IL-6 alone, IL-6 failed to accumulate into cells (FIG. 12A), the addition of BsAb allowed IL-6 accumulation in MCF7 at 307° C., this was not the case at 4° C. (FIG. 12B).

### Effect of Bispecific Antibody on IL-6 Dependent (XG6) and IL-6 Independent (Raji) Cell Lines Viability

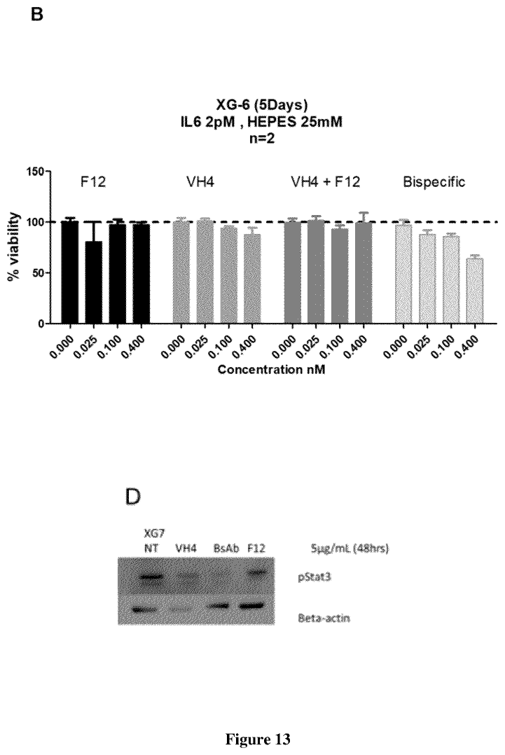

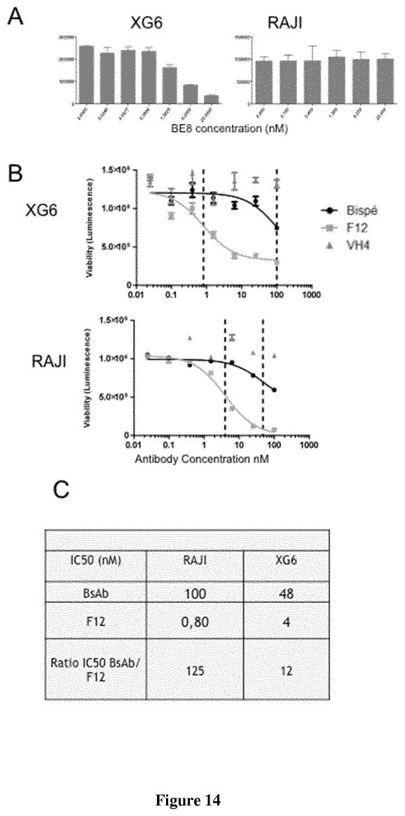

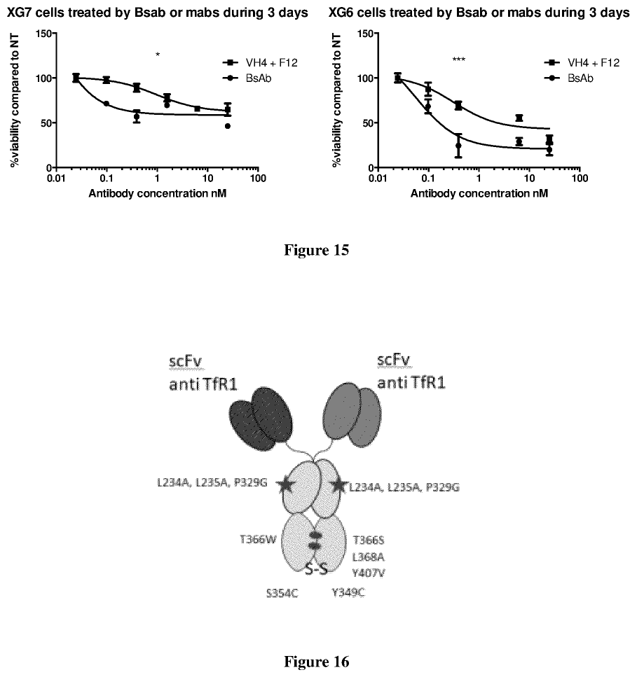

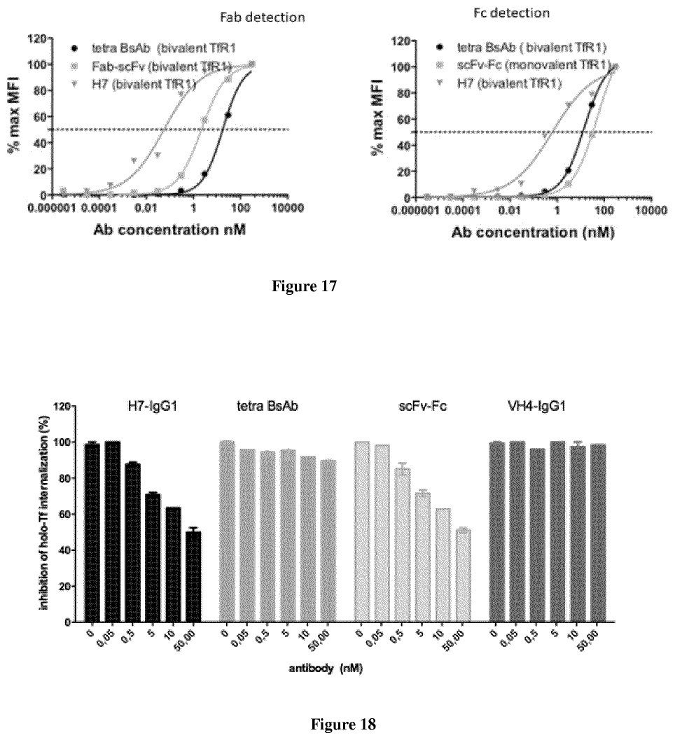

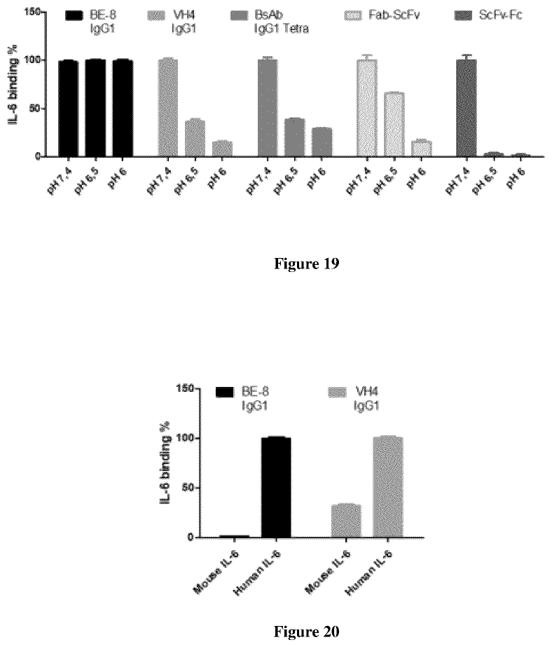

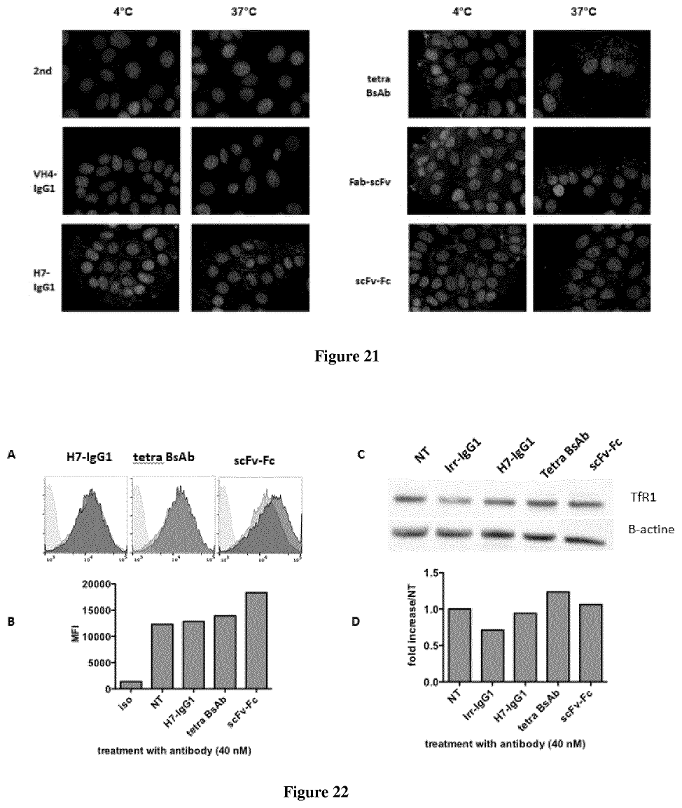

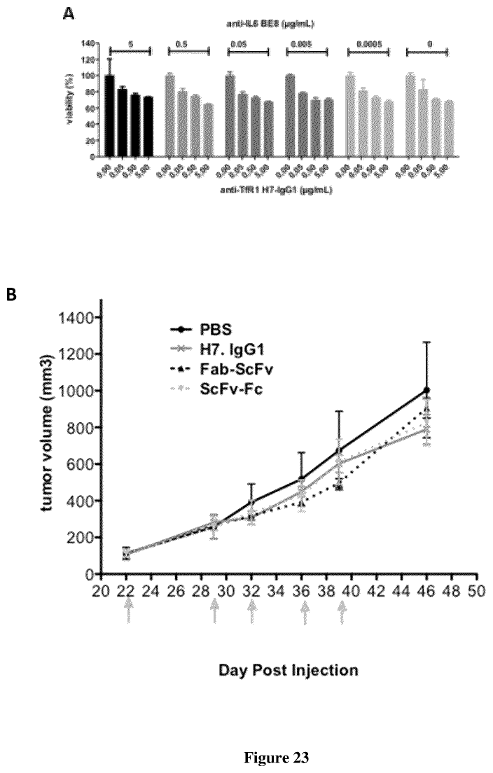

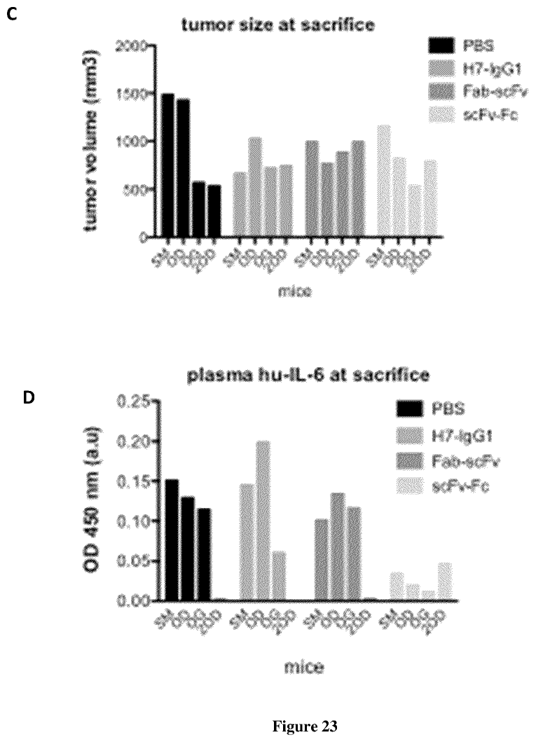

Effect of bispecific antibody compared to monoclonal combination on XG6 cells Various antibody combinations were tested on the XG6 cell line with concentrations ranging from 70 pM to 70 nM of antibodies for 5 days. A strong effect was observed with the monoclonal anti-TfR1 F12 (IC50 of 4 nM) which is an iron deprivating antibody, compared with the BsAb (IC50=50 nM). The VH4 anti-IL-6 antibody, as expected, had limited effect since it only partially neutralizes IL-6 (Devanaboyina et al., 2013). Consistently, the combination of the 2 parental anti-TfR1 F12 and anti-IL-6 VH4 antibodies had similar effect than the antibody F12 alone. However, it was noticeable that the BsAb had a better inhibitory effect than the monoclonal combination at concentrations below 1 nM (FIG. 13A).

To confirm this effect, XG6 cells were treated with reduced IL6 concentration (4 pM), and low concentrations of antibodies. The superiority of the BsAb compared to monoclonal combination was confirmed (FIG. 13B, 13C) when used at concentration of 400 pM. Additionally BsAb inhibited the IL-6/JAK2/STAT-3 pathway as shown by reduced STAT-3 phosphorylation on XG6 BsAb treated cells (FIG. 13D in XG7 cells).

Compared Effect of bsAb on XG6 Cells and Raji Cell Lines

Iron deprivation and IL-6 deprivation likely both account for the effect of the BsAb on XG6. Indeed, we found that the BsAb was more active on the IL-6 dependent XG6 cell line than on the IL-6 independent Raji cell line (IC₅₀ of 50 nM versus 100 nM, respectively), beside the fact the parental mAb anti-TfR1 F12 was less active on XG6 than Raji cells (IC₅₀ of 4 nM versus 0.8 nm, respectively) (FIG. 14). This indirectly observation comfort the demonstration that the BsAb has a sweeping activity.

### Compared Effect of BsAb on XG6 Cells and XG7 Myeloma Cell Lines (Both IL-6 Dependent) in Low Serum Condition (FCS 1%)

IL6 dependent myeloma cell lines (XG6 and XG7) were treated with BsAb or parental mAbs combination for 3 days in RPMI medium containing 1% FCS, HEPES 25 mM and IL6 at 4 pM. The FCS proportion was reduced to 1% in order to (1) limit cell proliferation due to FCS growth factors and therefore to reduce the impact of iron deprivation mediated by the anti-TfR1 paratope of the BsAb or by the parental F12 mAb, and (2) to make the cells mainly dependent of IL-6 for their survival, and not on other growth factors to enhance IL-6 deprivation effect.

In these conditions, BsAb shows a better effect compared to parental mAb combination both on XG6 and XG7 cells (FIG. 15). A maximal inhibition of 50% is obtained in XG7 cell lines and 80% in XG6 cell line. The IC₅₀ of BsAb (concentration required for 50% of the maximal effect) is approximatively 0.1 nM for both cell lines. The IC₅₀ of the antibody combination, whose effect, as VH4 is a non neutralizing anti-IL-6 mAb, is only due to iron deprivation by the anti-TfR1 F12, is 1 log higher (1 nM). Treatments with the combination versus BsAb are significantly different (ANOVA 2, p-value of 0.0161 for XG7 and p-value<0.0001 for XG6 cells).

### Example 8: Bispecific Antibodies Affinity for Native TfR1

### Method

The 3 formats have various valency for TfR1 or IL-6 (monovalency or bivalency) and comprise a Fc region (scFv-Fv, BsAb) or not (Fab-scFv). The affinity for native TfR1 has been tested for these 3 formats.

RAJI cells are incubated at 4° C., 1 hour in the presence of an increasing dose of each bispecific antibody (Fab-scFv; scFv-Fc; IgG1 tetravalent) and the H7.IgG1 antibody is used in positive control. Bispecific antibodies are detected either by a secondary antibody against human IgG Fab specific-FITC (SIGMA-F5512, 4° C., 1 h) or by an antibody against human IgG Fc-specific-FITC (SIGMA F9512, 4° C., 1 h). The fluorescence (MFI) of the cells is then measured by flow cytometry.

### Conclusion

The 3 formats have an apparent affinity for TfR1.

H7 IgG1 (bivalent for TfR1) has an apparent affinity (EC50 0.5 nM) with both detection system. Fab-scFv antibody (bivalent for TfR1) has an apparent affinity slightly lower than that of H7.IgG1 (EC50 2.2 nM). The tetravalent BsAb (bivalent for TfR1) and scFv-Fc (monovalent for TfR1) have the lowest apparent affinities 11.6 nM and 30.1 nM, respectively (FIG. 17).

### Example 9: Inhibition of Holo-tTf Internalization by the Three Bispecific Antibodies

### Method

RAJI B lymphoma cells grown in their culture medium (10% SVF) are co-incubated with a fixed concentration of Holo-Tf conjugated to Alexa 488 (500 nM) (Thermofisher, transferrin from human serum, Alexa fluor™ 488 conjugate t13342) and increasing concentrations of bispecific antibodies or with H7-IgG1 (positive control) or with anti-IL-6 VH4-IgG1 (negative control) for 3 hours at 37° C.

After 3 hours of incubation, RAJI cells are washed (PBS 1% fetal calf serum) and the fluorescence is measured. The fluorescence is expressed as a percentage compared to the Mean Fluorescence intensity (MFI) obtained without antibodies.

At 37° C., H7-IgG1 is blocking the internalization of Holo-Tf in a dose-dependent manner (50% of internalization blockade with 50 nM of H7 IgG1). The Fab-scFv format (bivalent for TfR1) blocks the internalization of Holo-Tf in a manner equivalent to the parent antibody H7 IgG1. Surprinsingly, the scFv-Fc format (monovalent for TfR1 binding) inhibits Holo-Tf internalization as strongly as the bivalent IgG and Fab-scFv formats similarly at high and low concentrations while the tetravalent BsAb IgG1 tetra shows a limited blockade of internalization of holo-Tf (only 10% inhibition at 50 nM) (FIG. 18).

### Conclusion:

The 3 formats block the internalization of holo-Tf.

The tetra BsAb blocks the internalization of holo-Tf at the maximal concentration used in this assay (50 nM). Of note holo-Tf total concentration in this experiment is estimated at 1 500 nM (500 nM human holo-Tf-A488 combined to 1 pM bovine holo-Tf present in the fetal calf serum).

Surprisingly and despite a lower apparent affinity for native TfR1, the scFv-Fc format (monovalent for TfR1) blocks TfR1 internalization as strongly as the bivalent formats H7-IgG1 and Fab-scFv.

### Example 10: Bispecific Antibodies Binding to IL-6 at Various pH Methods

Recombinant IL-6 (Peprotech, 200-06) Ig/ml in PBS at pH 7.4 is adsorbed at night at 4° C. on an ELISA plate (invitrogen, Nunc Maxisorp™ flat-bottom) after 3 washes (PBS, pH 7.4 with 0.05% Tween-20) and saturation (PBS pH 7.4 with 1% BSA, 2 h at room temperature). Bispecific antibodies or control anti-IL-6 antibodies (BE-8 for and VH4) are prepared (40 nM) in phosphate buffer solutions with variable pH (Na2HPO4-NaH2PO4, 20 mM) and are incubated for 2 h at room temperature. Washing steps following are also carried out at the different pH. Finally the antibodies are detected at pH 7,4 either by the Fc region (anti-Fc goat antibody (human IgG)-HRP; sigma A0170) or by the Ckappa domain (anti-CK-HRP goat antibody; sigma A7164). After adding the chromogenic substrate, the color developed is measured by spectrophotometry at 450 nm. The result is expressed as a percentage of binding with respect to the binding condition at pH 7.4.

### Conclusion

All 3 bispecific antibodies bind IL-6 in a pH dependent manner like the parental anti-IL-6 VH4 antibody.

However, pH sensitivity of the binding is higher in the IL-6 scFv format (scFv-Fc) than in the Fab format (tetravalent BsAb and Fab-scFv).

At pH 6,5, IL-6 binding is not detected for the scFv-Fc format and binding is reduced compared to pH 7.4 for the Fab containing BsAbs like for the parent anti-IL-6 IgG (FIG. 19).

### Example 11: Bispecific Antibodies Cross-Reactivity to IL-6

### Methods

Recombinant mouse IL-6 (Peprotech, 216-16) or human IL-6 (Peprotech, 200-06) are adsorbed (1 g/ml) over night at 4° C. on ELISA plates (Invitrogen, Nunc Maxisorp™ flat-bottom). After washing (PBS with 0.05% Tween-20) and saturation (PBS with 1% BSA), the antibodies BE8 or VH4 (40 nM, quadriplicates) were incubated at 7.4 pH for two hours. Finally the antibodies were detected by their Fc region with an anti-human IgG-Fc specific antibody conjugated to HRP (Sigma A0170) for VH4 and with an anti-mouse IgG-Fc specific antibody conjugated to HRP (a9044) for BE8. The VH4 antibody display cross-reactivity to mouse IL-6 but not BE8.

### Conclusion

The scFv-Fc format is cross-reactive to mouse IL-6 (FIG. 20) and to mouse TfR1 (not shown) and could be tested in fully mouse models of pathologies.

### Example 12: Internalization Properties of Three Bispecific Antibodies

### Methods:

Detection of the internalization of bispecific antibodies on the MCF7 breast cancer cell line by immunofluorescence (IL-6R negative).

20.000 cells are seeded on a glass slide in 24 wells. MCF7 are cultured in DMEM 10% serum. After 48 hours, cells are incubated with 30 μg/ml of bispecific antibodies (tetra-BsAb, Fab-scFv, scFv-Fc) or control antibodies (VH4-anti-IL-6 IgG1 or H7-anti-TfR1 IgG1) at 4° C. or 37° C., during 1 hour. Cells are then washed twice with PBS-0.1% Tween and once with PBS. Cells are then fixed with 3.7% formaldehyde for 40 minutes at room temperature. A new step of wash is carried out and cells are incubated during 50 min with PBS/BSA 2 mg/mL, 1 mL/well at 37° C. or 4° C.

Finally, bispecific antibodies or control antibodies are detected with an anti-Fc (human IgG1)-FITC (F9512 for BsAb IgG1 tetra, scFv-Fc and parental antibodies) or by a human anti-Fab FITC (F5512 for Fab-scFv). Incubation with a mix of secondary antibodies shows no background staining.

### Conclusion

The bispecific formats are able to bind TfR1 on cells surface at 4° C. and being internalized in cells are 37° C., like the parental mAbs H7 (FIG. 21). This result shows also that the valence for TfR1 (monovalence, bivalence) or the anti-TfR1 arm format (scFv or Fab) does not impact the internalization properties under these experimental condition. The same results were found in the IL-6 expressing CFPAC pancreatic cancer cell line (not shown).

### Example 13: TfR1 Modulation by the Bispecific Antibodies Format

In this example TfR1 modulation by the bispecific antibodies format has been evaluated by measurement of TfR1 protein level expression on XG-6 cells treated after 48 hours of treatment with bispecific formats or parental antibody H7-IgG1.

Two hundred thousand XG-6 cells/mL cells are prepared in a final volume of 4 mL (RPMI 5% IL-6 2 ng/mL for XG-6) and treated in the presence of 40 nM bispecific antibodies or parental antibodies. This cell line was chosen because it expresses IL-6R in addition to TfR1, that could influence the trafficking of TfR1 in case it interacts with both TfR1 and IL-6/IL-6R at the same time (of note, Binding to IL-6R is not prevented by the VH4 parental antibody).

For surface TfR1 expression (FIG. 22 A, B), membrane expression of TfR1 after 48 hours of treatment with bispecific antibodies or controls is measured by FACS analysis. After 48 h, 300,000 cells are collected and TfR1 expression is measured by a human anti-TfR1 mouse antibody coupled to AlloPhycoCyanin (APC) (BD 551374). BD 551374's binding site is different from H7 binding's site, TfR1 expression can be monitored independently of H7-IgG1 or BsAbs in the assay. The antibody APC mouse igg2a, κ isotype control (BD 551414) is used as a negative control. Graphs in FIG. 22A represent histogram overlays of non-stained (light grey), or TfR1 stained (medium grey) non treated cells, and from left to right (dark grey), TfR1 staining of H7-IgG1, tetra-BsAb or scFv-Fc treated cells, respectively. In FIG. 22 B, the MFI value is represented in function of the treatment.

For total TfR1 level quantification (FIG. 22 C,D), cells are collected after 48 h. and washed once with cold PBS, protein are then extracted with a laemli buffer 1× (without bromophenol blue) and quantified. 30 g of protein are used for Western-Blot. In FIG. 22C, TfR1 and Beta-actine are detected by a primary mouse antibody (thermo 136800 for TfR1, cell signaling 8 h10d10 for Beta-actin) on the night at 4° C. then detection is performed with a secondary antibody (sigma a3673) anti-mouse (γ-chain specific) coupled HRP (1 h at room temperature). The quantification (FIG. 22 D) is done using the software imageJ: TfR1 protein bands are normalized to Beta-actin protein bands. Finally fold change ratio is calculated.

### Conclusion:

The bispecific antibodies Tetra BsAb and scFv-Fc do not induce degradation of TfR1.

On XG6, H7 IgG1 does not decrease TfR1 expression (total and surface) after 48 h of treatment, despite its high internalization properties and TfR1 level of cells treated by tetra BsAb do not vary significantly compared to untreated cells. When cells are treated with scFv-Fc, TfR1 surface levels show a strong increase compared to untreated cells while total levels are slightly increased. The same modulation of TfR1 was also observed on Raji cells treated with the scFv-Fc (increase of surface TfR1, both surface and total) (not shown).

### Example 14: Characterization of Bispecific Antibodies Sweeping Activity In Vivo

The CFPAC cell line was chosen because it secretes IL-6 and is moderately susceptible to iron deprivation. Panel A of FIG. 23 shows a maximum of 30% on decrease of viability after 5 days of incubation with anti-TfR1 H7-IgG1. Viability is not affected by the combination with IL-6 deprivation using the anti-IL-6 neutralizing antibody BE8 or by incubation with the anti-IL6 alone. In this experiment, 4000 CFPAC cells were seeded in 96-well plates (100 μL IMDM 10% foetal calf serum), one day after, 100 μL of antibody solution was added. After 5 more days, viability was quantified by a MTS assay (Celltiter 96® AqueousOne solution Cell proliferation assay (PROMEGA G3580). Therefore, the CFPAC cell line is moderately sensitive to iron deprivation and not sensitive to IL-6 deprivation.

Then, 16 nude mice were xenografted subcutaneously with 3 million of CFPAC cells. After 22 days of tumor development, mice were divided in 4 groups of homogeneous tumor size (100 mm3). Then mice were treated for 3.5 weeks (5 mg/kg, i.p, light grey arrow, D22, D29, D32, D36 and D39) either with H7 IgG1 parental monoclonal antibody or with the Fab-scFv or the scFv-Fc and were sacrificed at day 46. The FIG. 22 B represents the average of tumor sizes in each group at different time (days). FIG. 22C represents tumor size in each treatment group. At the time of sacrifice, human specific IL-6 quantification in the plasma was performed by ELISA (900-T16 peprotech). Samples were 1/5 and tested in duplicate. Results are represented in crude OD at 450 nm, background OD corresponding to naïve mice serum diluted the same was subtracted from the values indicated. Mice treated with the bispecific antibody scFv-Fc show clearly a decrease in plasma IL-6 compared to other groups, showing that this format efficiently depletes human IL-6 in vivo.

### Conclusion

The growth of the tumor is not affected significantly by the treatments in this experiment.

The quantification by ELISA of human IL-6 in the plasma originating from CFPAC tumors of similar size will allow showing a sweeping effect in vivo. Mice treated with the bispecific antibody scFv-Fc show clearly a decrease in plasma IL-6 compared to other groups, showing that this format efficiently depletes human IL-6 in vivo (FIG. 22D).

This result support also the idea that for a better sweeping effect based on TfR1, it is interesting to have a valency of 1 for each targets:

For a same number of TfR1 receptor at cell surface, a higher proportion of scFv-Fc will be bound compare to Fab-scFv (which is bivalent for TfR1). As a consequence more IL-6 will be bound by scFv-Fc and sweeped.

The tetra BsAb was not tested in this experiment.

1-16. (canceled)

17. A bispecific antibody comprising:
a first antigen-binding domain which binds competitively to TfR1; and
a second antigen-binding domain which binds to a soluble antigen, wherein the soluble antigen dissociates from the second antigen-binding domain into endosome.

18. The bispecific antibody according to claim 17, wherein the soluble antigen is a pro-tumoral factor.

19. The bispecific antibody according to claim 18, wherein the pro-tumoral factor is selected among cytokines, growth factors, matrix metalloproteinases (MMPs) family, urokinase-type plasminogen activator, soluble E-selectin, cyclooxygenases, hepatitis B virus X protein, Nonstructural proteins of Hepatitis C virus, prostaglandin, chemokines, Galectin-3 binding protein, Bcl-2-associated athanogene 3, PD-L1.

20. The bispecific antibody according to claim 18, wherein the pro-tumoral factor is selected from the group consisting of IL-6, PDL-1, GM-CSF, Gal-3BP, BAG3, IL-17 family, EGF, NRG1, NRG2, NRG3, NRG4, HGF, and RANK ligand.

21. The bispecific antibody according to claim 17, wherein the soluble antigen is a pro-inflammatory factor.

22. The bispecific antibody according to claim 20, wherein the pro-inflammatory factor is selected from the group consisting of cytokines, C5, growth factors and IgE.

23. The bispecific antibody according to claim 20, wherein the pro-inflammatory factor is selected from the group consisting of IL-6, TNF-α, soluble TNF-α receptor, IL-1β, IL-5, IL-17A, IL-12, IL-23, C5, BAFF, IgE and TGFβ.

24. The bispecific antibody according to claim 17, wherein the first antigen-binding domain which binds competitively to TfR1 does not induce degradation of the TfR1.

25. A pharmaceutical composition comprising a bispecific antibody according claim 17.

26. A method of eliminating a soluble antigen from the circulation or from the tumor or from an inflamed zone comprising a step of administering a bispecific antibody according to claim 17.

27. A method of treating a patient suffering from cancer by administering to the patient an effective amount of a bispecific antibody according to claim 17.

28. The method of treating according to claim 27, wherein the cancer is a solid cancer or a multiple myeloma.

29. The method of treating according to claim 28, wherein the solid cancer is selected among pancreatic cancer, neuroblastoma, leukemia, lymphoma, breast cancer, cancer related cachexia, gastrointestinal cancer, lung cancer, melanoma, ovarian cancer, prostate cancer, renal cancer, hepatocarcinoma

30. A method of treating a patient suffering from inflammatory pathology by administering to the patient an effective amount of a bispecific antibody according to claim 17.

31. A method of treating according to claim 30, wherein the inflammatory pathology is selected from inflammatory bowel disease; psoriasis; rheumatologic inflammatory pathologies such as rheumatoid arthritis, ankylosing spondylitis; multiple sclerosis; autoimmune pathologies such as systemic lupus, erythematosus neuromyelitis optica; asthma; and allergies.
